(19) United States
(12) Patent Application Publication
(10) Pub. No.: US 2023/0263786 A1
(43) Pub. Date: Aug. 24, 2023

(54) MODULATION OF DRUG-DRUG INTERACTIONS OF VADADUSTAT

(71) Applicants: Akebia Therapeutics, Inc., Cambridge, MA (US); Mitsubishi Tanabe Pharma Corporation, Osaka-shi (JP)

(72) Inventors: Ajit CHAVAN, Cambridge, MA (US); Rishikesh SAWANT, Cambridge, MA (US); Yoshimasa KOKADO, Osaka-shi (JP); Takehiro NANJO, Osaka-shi (JP); Shuji KINOSHITA, Osaka-shi (JP); Naomi KONISHI, Osaka-shi (JP)

(73) Assignees: Akebia Therapeutics, Inc., Cambridge, MA (US); Mitsubishi Tanabe Pharma Corporation, Osaka-shi (JP)

(21) Appl. No.: 18/001,917

(22) PCT Filed: Jun. 17, 2021
(86) PCT No.: PCT/US2021/037781

§ 371 Date: Dec. 15, 2022

Related U.S. Application Data
(60) Provisional application No. 63/041,226, filed on Jun. 19, 2020.
(60) Provisional application No. 63/041,308, filed on Jun. 19, 2020.
(60) Provisional application No. 63/155,013, filed on Mar. 1, 2021.

(51) Int. Cl. A61K 31/4418; A61K 33/06; A61K 33/244; A61K 33/26; A61K 31/366; A61K 31/47; A61K 31/405; A61K 31/22; A61K 31/505; A61K 31/40

(52) U.S. Cl. CPC A61K 31/4418; A61K 33/06; A61K 33/244; A61K 33/26; A61K 31/366; A61K 31/47; A61K 31/405; A61K 31/22; A61K 31/505; A61K 31/40

(57) ABSTRACT

Provided herein are methods for reducing, minimizing, or controlling drug-drug interactions resulting from administration of a first drug that is vadadustat (i.e., {[5-(3- chlorophenyl)-3-hydroxypyridine-2-carbonyl]amino}acetic acid (Compound 1)) and a second drug (for example, a drug comprising a multivalent cation such as calcium, iron, magnesium, lanthanum, aluminum, and the like; a statin drug; sulfasalazine; or furosemide), to a subject.

### CROSS-REFERENCE TO RELATED APPLICATIONS

The present application claims benefit of U.S. Provisional Application No. 63/041,226, filed Jun. 19, 2020; U.S. Provisional Application No. 63/041,308, filed Jun. 19, 2020; and U.S. Provisional Application No. 63/155,013, filed Mar. 1, 2021, each of which is incorporated by reference in its entirety.

### BACKGROUND OF THE INVENTION

{[5-(3-chlorophenyl)-3-hydroxypyridine-2-carbonyl]amino}acetic acid (Vadadustat, Code: AKB-6548, Compound 1), having a chemical formula:

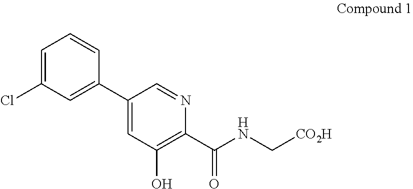 [SMILES: O=C(O)CNC(=O)c1ncc(-c2cccc(Cl)c2)cc1O]

is prolyl hydroxylase inhibitor, and may be administered to subjects to treat or prevent diseases ameliorated by modulation of hypoxia-inducible factor (HIF) prolyl hydroxylase (e.g., Peripheral Vascular Disease (PVD), Coronary Artery Disease (CAD), heart failure, ischemia, hypoxia, and anemia). For some subjects receiving Compound 1, this compound may be administered as part of a therapeutic regimen that includes other medications and therapeutic agents. For example, some subjects being administered Compound 1 also require therapeutic agents such as a drug comprising a multivalent cation (e.g., iron supplementation), statin drugs, sulfasalazine, or furosemide. The bioavailability of Compound 1 and/or the other medications and therapeutic agents (e.g., multivalent cation-containing compositions, statin drugs, sulfasalazine, or furosemide) potentially may be affected when co-administered to the subjects. Accordingly, new methods are needed for controlling any such drug-drug interactions when administering Compound 1 with other medications and therapeutic agents (e.g., multivalent cation-containing compositions, statin drugs, sulfasalazine, or furosemide) so as not to adversely impact the therapeutic effects of an administered drug or to adversely impact a subject.

### SUMMARY

The invention is based, in part, on the surprising discovery of new therapeutic regimens that result in the modulation of drug-drug interactions (e.g., preventing, controlling, reducing, or minimizing drug-drug interactions) between a first drug that is vadadustat and a second drug (e.g., drugs comprising a multivalent cation such as calcium, iron, magnesium, lanthanum, aluminum, and the like; statin drugs; sulfasalazine; or furosemide). Such regimens can have beneficial outcomes for a patient, including those described herein.

In one aspect, the present invention provides herein a method of preventing, controlling, reducing, or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug comprises multivalent cations,
wherein the subject has renal anemia (anemia secondary to or associated with chronic kidney disease), and
wherein (a) is given at least 1 hour or at least 2 hours before and/or after taking (b).

In embodiments, (b) is a calcium-containing composition, magnesium-containing composition, iron-containing composition, lanthanum-containing composition, or aluminum-containing composition.

In another aspect, the present invention provides herein a method of preventing, controlling, reducing, or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is an iron-containing composition, wherein the subject has renal anemia (anemia secondary to or associated with chronic kidney disease), and
wherein (a) is given at least 1 hour or at least 2 hours before and/or after taking (b).

In another aspect, the present invention provides a method of increasing or maintaining bioavailability of a drug comprising administering to the subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

In embodiments, (b) is a calcium-containing composition, magnesium-containing composition, iron-containing composition, lanthanum-containing composition, or aluminum-containing composition.

In another aspect, the present invention provides a method of increasing or maintaining bioavailability of a drug comprising administering to the subject:
(a) an effective amount of the drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

In another aspect, the present invention provides a method of minimizing, controlling, or preventing a decrease in the absorption of a drug comprising administering to the subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

In embodiments, (b) is a calcium-containing composition, magnesium-containing composition, iron-containing composition, lanthanum-containing composition, or aluminum-containing composition.

In another aspect, the present invention provides a method of minimizing, controlling, or preventing a decrease in the absorption of a drug comprising administering to the subject:
(a) an effective amount of the drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

In another aspect, the present invention provides a method of controlling, minimizing, reducing, or preventing drug-multivalent cation chelate formation comprising administering to a subject:
(a) an effective amount of the drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

In embodiments, (b) is a calcium-containing composition, magnesium-containing composition, iron-containing composition, lanthanum-containing composition, or aluminum-containing composition.

In another aspect, the present invention provides a method of controlling, minimizing, reducing, or preventing drug-iron chelate formation comprising administering to a subject:
(a) an effective amount of the drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

In embodiments, (a) is given at least 1 hour before and/or after taking (b).

In embodiments, (a) is given at least 2 hours before and/or after taking (b).

In embodiments, (b) is an iron-containing composition that is oral iron or an iron-containing phosphorus adsorbent.

In embodiments, (b) is an iron-containing composition that is an iron-containing phosphorus adsorbent.

In embodiments, the iron-containing composition comprises one or more of ferrous sulfate (also referred to as iron sulfate or iron (II) sulfate), sodium ferrous citrate, ferric citrate, or sucroferric oxyhydroxide.

In embodiments, administration of the iron-containing composition is associated with a medical treatment.

In embodiments, the iron-containing composition is administered as a supplement.

In embodiments, the iron-containing composition is administered as a tablet.

In embodiments, the iron-containing composition tablet is a slow release tablet.

In embodiments, the iron-containing composition tablet is a chewable tablet.

In embodiments, (b) is a calcium-containing composition.

In embodiments, (b) is oral calcium acetate.

In embodiments, (b) is oral calcium carbonate.

In embodiments, (b) is a lanthanum-containing composition.

In embodiments, (b) is oral lanthanum carbonate.

In embodiments, (b) is a magnesium-containing composition.

In embodiments, (b) is an aluminum-containing composition.

In embodiments, {[5-(3-chlorophenyl)-3-hydroxypyridine-2-carbonyl]amino}acetic acid is administered at least 2 hours before the iron-containing composition.

In embodiments, {[5-(3-chlorophenyl)-3-hydroxypyridine-2-carbonyl]amino}acetic acid is administered at least 1 hour before the iron-containing composition.

In embodiments, {[5-(3-chlorophenyl)-3-hydroxypyridine-2-carbonyl]amino}acetic acid is administered at least 2 hours after the iron-containing composition.

In embodiments, {[5-(3-chlorophenyl)-3-hydroxypyridine-2-carbonyl]amino}acetic acid is administered at least 2 hours before the calcium-containing composition.

In embodiments, {[5-(3-chlorophenyl)-3-hydroxypyridine-2-carbonyl]amino}acetic acid is administered at least 1 hour before the calcium-containing composition.

In embodiments, {[5-(3-chlorophenyl)-3-hydroxypyridine-2-carbonyl]amino}acetic acid is administered at least 2 hours after the calcium-containing composition.

In embodiments, {[5-(3-chlorophenyl)-3-hydroxypyridine-2-carbonyl]amino}acetic acid is administered at least 2 hours before the lanthanum-containing composition.

In embodiments, {[5-(3-chlorophenyl)-3-hydroxypyridine-2-carbonyl]amino}acetic acid is administered at least 1 hour before the lanthanum-containing composition.

In embodiments, {[5-(3-chlorophenyl)-3-hydroxypyridine-2-carbonyl]amino}acetic acid is administered at least 2 hours after the lanthanum-containing composition.

In another aspect, the present invention provides a method of treating renal anemia (anemia secondary to or associated with chronic kidney disease), comprising administering to a subject having renal anemia an effective amount of a compound which is {[5-(3-chlorophenyl)-3-hydroxypyridine-2-carbonyl]amino}acetic acid or pharmaceutically acceptable salt, solvate, or hydrate thereof,

wherein the compound is for administering together with one or more of oral iron-containing composition selected from the group consisting of sodium ferrous citrate, ferric citrate, and sucroferric oxyhydroxide.

In another aspect, the present invention provides a method of treating renal anemia (anemia secondary to or associated with chronic kidney disease), comprising administering to a subject having renal anemia an effective amount of a compound which is {[5-(3-chlorophenyl)-3-hydroxypyridine-2-carbonyl]amino}acetic acid or pharmaceutically acceptable salt, solvate, or hydrate thereof,

wherein the compound is for administering together with an oral calcium-containing composition that is calcium acetate or calcium carbonate.

In another aspect, the present invention provides a method of treating renal anemia (anemia secondary to or associated with chronic kidney disease), comprising administering to a subject having renal anemia an effective amount of a compound which is {[5-(3-chlorophenyl)-3-hydroxypyridine-2-carbonyl]amino}acetic acid or pharmaceutically acceptable salt, solvate, or hydrate thereof,

wherein the compound is for administering together with an oral lanthanum-containing composition that is lanthanum carbonate.

In another aspect, the present invention provides herein a method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is a statin drug,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is a statin drug,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is a statin drug,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of maintaining bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a statin drug,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of minimizing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a statin drug,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of preventing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a statin drug,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of controlling an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a statin drug,
wherein the subject has renal anemia (anemia secondary to or associated with chronic kidney disease), and
wherein the amount of (b) is adjusted compared to the amount when administered in the absence of (a) or in monotherapy.

In embodiments, the subject is at risk of or has cardiovascular disease or diabetes.

In embodiments, the subject has dyslipidemia.

In embodiments, the subject has elevated cholesterol or elevated triglycerides (hypertriglyceridemia).

In embodiments, the subject has elevated total cholesterol, elevated LDL-cholesterol.

In embodiments, the subject has low HDL-cholesterol.

In embodiments, the statin drug of (b) is simvastatin, pitavastatin, fluvastatin, lovastatin, pravastatin, rosuvastatin, or atorvastatin.

In embodiments, the amount of (b) is decreased compared to the amount when administered in the absence of (a) or in monotherapy.

In embodiments, the amount of (b) is decreased by about 20% to about 80% compared to the amount when administered in the absence of (a) or in monotherapy.

In embodiments, the amount of (b) is decreased by about 40% to about 60% compared to the amount when administered in the absence of (a) or in monotherapy.

In embodiments, a drug-drug interaction is between Compound 1 and atorvastatin as administered. In embodiments, a drug-drug interaction is between Compound 1 and one or more metabolites of atorvastatin. In embodiments, a metabolite of atorvastatin is o-hydroxy atorvastatin and/or p-hydroxy atorvastatin.

In embodiments, the method relates to the exposure to atorvastatin as administered. In embodiments, the method relates to the exposure to one or more metabolites of atorvastatin. In embodiments, a metabolite of atorvastatin is o-hydroxy atorvastatin and/or p-hydroxy atorvastatin.

In embodiments, the statin is rosuvastatin, and the dose is decreased by at least about 2.5, 5, or 10 mg.

In embodiments, the maximum daily dose of rosuvastatin is about 10 mg.

In embodiments, the statin is simvastatin, and the dose is decreased by at least about 5, 10, or 20 mg.

In embodiments, the maximum daily dose of simvastatin is about 20 mg.

In embodiments, a drug-drug interaction is between Compound 1 and simvastatin as administered. In embodiments, a drug-drug interaction is between Compound 1 and one or more metabolites of simvastatin. In embodiments, a metabolite of simvastatin is p-hydroxysimvastatin acid.

In embodiments, the method relates to the exposure to simvastatin as administered.

In embodiments, the method relates to the exposure to one or more metabolites of simvastatin. In embodiments, a metabolite of simvastatin is p-hydroxysimvastatin acid.

In embodiments, the subject initially receives a statin that is rosuvastatin or simvastatin.

In embodiments, administration of rosuvastatin or simvastatin is discontinued.

In embodiments, (b) is administered simultaneously with (a). In embodiments, (b) is not administered simultaneously with (a).

In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid.

In embodiments, the daily dose of {[5-(3-chlorophenyl)-3-hydroxypyridine-2-carbonyl]amino}acetic acid is increased.

In embodiments, the subject is administered an initial daily dose of about 300 mg of {[5-(3-chlorophenyl)-3-hydroxypyridine-2-carbonyl]amino}acetic acid.

In embodiments, the subject is administered an initial daily dose of about 450 mg of {[5-(3-chlorophenyl)-3-hydroxypyridine-2-carbonyl]amino}acetic acid.

In embodiments, the subject is administered an initial daily dose of about 600 mg of {[5-(3-chlorophenyl)-3-hydroxypyridine-2-carbonyl]amino}acetic acid.

In another aspect, the present invention provides herein a method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

In embodiments, a drug-drug interaction is between Compound 1 and sulfasalazine as administered. In embodiments, a drug-drug interaction is between Compound 1 and one or more metabolites of sulfasalazine. In embodiments, a metabolite of sulfasalazine is sulfapyridine and/or mesalamine.

In another aspect, the present invention provides herein a method of maintaining bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of minimizing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of preventing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of controlling an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

In embodiments, the method relates to the exposure to sulfasalazine as administered. In embodiments, the method relates to the exposure to one or more metabolites of sulfasalazine. In embodiments, a metabolite of sulfasalazine is sulfapyridine and/or mesalamine.

In embodiments, the subject is at risk of or has ulcerative colitis, Crohn's disease, or rheumatoid arthritis.

In embodiments, (b) is administered simultaneously with (a). In embodiments, (b) is not administered simultaneously with (a).

In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid.

In embodiments, the daily dose of {[5-(3-chlorophenyl)-3-hydroxypyridine-2-carbonyl]amino}acetic acid is increased.

In embodiments, the subject is administered an initial daily dose of about 300 mg of {[5-(3-chlorophenyl)-3-hydroxypyridine-2-carbonyl]amino}acetic acid.

In embodiments, the subject is administered an initial daily dose of about 450 mg of {[5-(3-chlorophenyl)-3-hydroxypyridine-2-carbonyl]amino}acetic acid.

In embodiments, the subject is administered an initial daily dose of about 600 mg of {[5-(3-chlorophenyl)-3-hydroxypyridine-2-carbonyl]amino}acetic acid.

In another aspect, the present invention provides herein a method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is a diuretic that is furosemide,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is a diuretic that is furosemide,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is a diuretic that is furosemide,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of maintaining bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a diuretic that is furosemide,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of minimizing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a diuretic that is furosemide,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of preventing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a diuretic that is furosemide,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of controlling an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a diuretic that is furosemide,
wherein the subject has renal anemia (anemia secondary to or associated with chronic kidney disease), and
wherein the amount of (b) is adjusted compared to the amount when administered in the absence of (a) or in monotherapy.

In embodiments, the subject is at risk of or has at risk of or has edema.

In embodiments, the subject has cardiovascular disease or liver disease.

In embodiments, the edema is induced by chronic kidney disease, cardiovascular disease, or liver disease.

In embodiments, (b) is administered simultaneously with (a). In embodiments, (b) is not administered simultaneously with (a).

In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid.

In embodiments, the daily dose of {[5-(3-chlorophenyl)-3-hydroxypyridine-2-carbonyl]amino}acetic acid is increased.

In embodiments, the subject is administered an initial daily dose of about 300 mg of {[5-(3-chlorophenyl)-3-hydroxypyridine-2-carbonyl]amino}acetic acid.

In embodiments, the subject is administered an initial daily dose of about 450 mg of {[5-(3-chlorophenyl)-3-hydroxypyridine-2-carbonyl]amino}acetic acid.

In embodiments, the subject is administered an initial daily dose of about 600 mg of {[5-(3-chlorophenyl)-3-hydroxypyridine-2-carbonyl]amino}acetic acid.

In another aspect, the present invention provides herein a method of treating renal anemia (anemia secondary to or associated with chronic kidney disease) in a subject with hepatic impairment, comprising administering to the subject an effective amount of {[5-(3-chlorophenyl)-3-hydroxypyridine-2-carbonyl]amino}acetic acid, or a pharmaceutically acceptable salt thereof.

In embodiments, the hepatic impairment is mild or moderate hepatic impairment.

In embodiments, the hepatic impairment is characterized as Child Pugh Class B (7-9 points).

In embodiments, the hepatic impairment is characterized by elevated bilirubin levels, decreased serum albumin levels, elevated international normalized ratios (INR), ascites, and/or hepatic encephalopathy.

In embodiments, the elevated international normalized ratio (INR) is >2.20.

In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid.

In embodiments, the daily dose of {[5-(3-chlorophenyl)-3-hydroxypyridine-2-carbonyl]amino}acetic acid is increased.

In embodiments, the subject is administered an initial daily dose of about 300 mg of {[5-(3-chlorophenyl)-3-hydroxypyridine-2-carbonyl]amino}acetic acid.

In embodiments, the subject is administered an initial daily dose of about 450 mg of {[5-(3-chlorophenyl)-3-hydroxypyridine-2-carbonyl]amino}acetic acid.

In another aspect, the present invention provides herein a method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of maintaining bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of minimizing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of preventing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

In another aspect, the present invention provides herein a method of controlling an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

In embodiments, the drug that is a BCRP (breast cancer resistance protein) substrate is mitoxantrone, imatinib, irinotecan, lapatinib, apixaban, atorvastatin, baricitinib, copanlisib, dolutegravir, eltrombopag, ethinylertradiol, glecaprevir, glyburide, letermovir, methotrexate, paritaprevir, pibrentasvir, pravastatin, presatovir, prucalopride, rosuvastatin, simvastatin, sofosbuvir, sulfasalazine, tenofovir, topotecan, velpatasvir, venetoclax, or voxilaprevir.

In embodiments, the drug that is a BCRP (breast cancer resistance protein) substrate is atorvastatin, pravastatin, rosuvastatin, simvastatin, or sulfasalazine.

In embodiments, (b) is administered simultaneously with (a). In embodiments, (b) is not administered simultaneously with (a).

In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid.

In embodiments, the daily dose of {[5-(3-chlorophenyl)-3-hydroxypyridine-2-carbonyl]amino}acetic acid is increased.

In embodiments, the subject is administered an initial daily dose of about 300 mg of {[5-(3-chlorophenyl)-3-hydroxypyridine-2-carbonyl]amino}acetic acid.

In embodiments, the subject is administered an initial daily dose of about 450 mg of {[5-(3-chlorophenyl)-3-hydroxypyridine-2-carbonyl]amino}acetic acid.

In embodiments, the subject is administered an initial daily dose of about 600 mg of {[5-(3-chlorophenyl)-3-hydroxypyridine-2-carbonyl]amino}acetic acid.

In another aspect, the present invention provides herein a method of treating renal anemia (anemia secondary to or associated with chronic kidney disease), in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving a drug that is a Breast Cancer Resistance Protein (BCRP) substrate.

In embodiments, the BCRP substrate is mitoxantrone, imatinib, irinotecan, lapatinib, apixaban, atorvastatin, baricitinib, copanlisib, dolutegravir, eltrombopag, ethinylertradiol, glecaprevir, glyburide, letermovir, methotrexate, paritaprevir, pibrentasvir, pravastatin, presatovir, prucalopride, rosuvastatin, simvastatin, sofosbuvir, sulfasalazine, tenofovir, topotecan, velpatasvir, venetoclax, or voxilaprevir.

In another aspect, the present invention provides herein a method of treating renal anemia (anemia secondary to or associated with chronic kidney disease), in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving a diuretic that is furosemide.

In another aspect, the present invention provides herein a method of treating renal anemia (anemia secondary to or associated with chronic kidney disease), in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving a statin drug.

In embodiments, the statin is simvastatin, pitavastatin, fluvastatin, lovastatin, pravastatin, rosuvastatin, or atorvastatin.

In another aspect, the present invention provides herein a method of treating renal anemia (anemia secondary to or associated with chronic kidney disease), in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving sulfasalazine.

In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid.

In embodiments, the daily dose of {[5-(3-chlorophenyl)-3-hydroxypyridine-2-carbonyl]amino}acetic acid is increased.

In embodiments, the subject is administered an initial daily dose of about 300 mg of {[5-(3-chlorophenyl)-3-hydroxypyridine-2-carbonyl]amino}acetic acid.

In embodiments, the subject is administered an initial daily dose of about 450 mg of {[5-(3-chlorophenyl)-3-hydroxypyridine-2-carbonyl]amino}acetic acid.

In embodiments, the subject is administered an initial daily dose of about 600 mg of {[5-(3-chlorophenyl)-3-hydroxypyridine-2-carbonyl]amino}acetic acid.

### BRIEF DESCRIPTION OF THE DRAWINGS

FIG. 1 shows mean (SD) plasma concentration of vadadustat when 1) administered alone; 2) administered concomitantly with calcium acetate (Day 3); 3) administered 1 hour before calcium acetate (Day 5); administered 2 hours after calcium acetate (Day 7).

FIG. 2 shows effect of a single dose of phosphate binder that is calcium acetate on the PK of vadadustat (300 mg). Values shown are geometric least squares mean ratios; the error bars represent the 90% geometric confidence interval. AUC_{0-∞}: area under the plasma concentration-time curve from time 0 to infinity; AUC₀₋ₗₐₛₜ: area under the plasma concentration-time curve from time 0 to last quantifiable concentration; Cₘₐₓ: maximum observed plasma concentration; PK: pharmacokinetics.

### DETAILED DESCRIPTION

Vadadustat ({[5-(3-chlorophenyl)-3-hydroxypyridine-2-carbonyl]amino}acetic acid; (Compound 1)) is a Hypoxia Inducible Factor Prolyl Hydroxylase inhibitor (HIF-PH inhibitor).

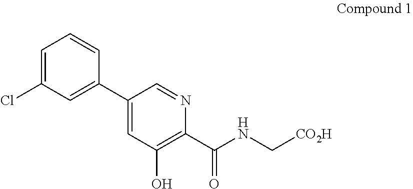 [SMILES: O=C(O)CNC(=O)c1ncc(-c2cccc(Cl)c2)cc1O]

Compound 1 has emerged as a new drug that is highly useful for treating or preventing renal anemia (anemia secondary to or associated with chronic kidney disease).

A subject with renal anemia (anemia secondary to or associated with chronic kidney disease) may be receiving other therapeutic agents concurrently with a vadadustat treatment regimen (e.g., to treat co-morbid conditions or to treat complications associated with chronic kidney disease or the renal anemia (anemia secondary to or associated with chronic kidney disease)). In particular, drug-drug interactions may occur when a patient is administered Compound 1 in combination with another drug (e.g., drugs comprising a multivalent cation such as calcium, iron, magnesium, lanthanum, aluminum, and the like; statin drugs; sulfasalazine; or furosemide).

A drug-drug interaction can manifest in various ways, including affecting pharmaceutical interactions, pharmacokinetic interactions, pharmacodynamic interactions, absorption, distribution, metabolism, or excretion. For example, a drug-drug interaction may adversely impact bioavailability and/or absorption of Compound 1 and/or the other therapeutic agents (e.g., drugs comprising a multivalent cation, statin drugs, sulfasalazine, furosemide or any other exemplary therapeutic agent described herein). In other embodiments, still other side-effects may result. Accordingly, modulation of a drug-drug interaction will be highly beneficial to achieving successful treatment of subject with chronic kidney disease or the renal anemia (anemia secondary to or associated with chronic kidney disease).

Provided herein are methods for reducing, minimizing, or controlling the drug-drug interactions resulting from administration of Compound 1 and other therapeutic agents, including drugs that comprise multivalent cations (e.g., an oral composition comprising calcium, iron, magnesium, lanthanum, aluminum, and the like, such as certain iron-containing compositions), statin drugs, sulfasalazine, and furosemide, to a subject. Also provided herein are methods for increasing and/or maintaining the bioavailability of Compound 1 and/or the other therapeutic agents, wherein Compound 1 and certain drugs (e.g., drugs comprising a multivalent cation such as calcium, iron, magnesium, lanthanum, aluminum, and the like; statin drugs; sulfasalazine; or furosemide) are administered to a subject.

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference for all purposes.

Animal: As used herein, the term “animal” refers to any member of the animal kingdom. In some embodiments, “animal” refers to humans, at any stage of development. In some embodiments, “animal” refers to non-human animals, at any stage of development. In embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Dose(s): As used herein, the term “dose(s)” means a quantity of the compound or a pharmaceutically acceptable salt, solvate, or hydrate thereof to be administered at one time. A dose may comprise a single unit dosage form, or alternatively may comprise more than a single unit dosage form (e.g., a single dose may comprise two tablets), or even less than a single unit dosage form (e.g., a single dose may comprise half of a tablet).

Daily dose: As used herein, the term “daily dose” means a quantity of the compound, or a pharmaceutically acceptable salt, solvate, or hydrate thereof that is administered in a 24-hour period. Accordingly, a daily dose may be administered all at once (i.e., once daily dosing) or alternatively the daily dosing may be divided such that administration of the compound is twice daily, three times daily, or even four times daily.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control sample or subject (or multiple control samples or subjects) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable”, as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C₁₋₄-alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quarternization of an amine using an appropriate electrophile, e.g., an alkyl halide, to form a quarternized alkylated amino salt.

Preventing: The term “prevent,” “preventing,” or “prevention,” as used herein refers to an effect that mitigates an undesired effect, e.g., an undesirable drug-drug interaction or the formation of a drug-iron chelate. Prevention does not require the 100% elimination of the possibility of an event. Rather, it denotes that the likelihood of the occurrence of the event has been reduced by the compound or method.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one-unit dose.

Treating: As used herein, the term “treat,” “treatment,” or “treating” refers to any method used to partially or completely alleviate, ameliorate, relieve, inhibit, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

As used herein, the term “HIF prolyl hydroxylase” is art-recognized and may be abbreviated as “PHD”. HIF prolyl hydroxylase is also known as “prolyl hydroxylase domain-containing protein” which may be abbreviated as “PHD”. In this regard, there are three different PHD isoforms, PHD1, PHD2, and PHD3, also referred to as EGLN2, EGLN1, and EGLN3, or HPH3, HPH2, and HPH1, respectively.

As used herein, the term “unit dosage form(s)” includes tablets; caplets; capsules, such as soft elastic gelatin capsules; sachets; cachets; troches; lozenges; dispersions; powders; solutions; gels; liquid dosage forms suitable for oral or mucosal administration to a patient, including suspensions (e.g., aqueous or non-aqueous liquid suspensions), emulsions (e.g., oil-in-water emulsions, or a water-in-oil liquid emulsion), solutions, and elixirs; and sterile solids (e.g., crystalline or amorphous solids) that can be reconstituted to provide liquid dosage forms suitable for oral or parenteral administration to a patient. The unit dosage form does not necessarily have to be administered as a single dose nor does a single unit dosage form necessarily constitute an entire dose.

As used herein, the term “oral iron” refers to iron-containing compositions that can be administered orally.

Metabolites of atorvastatin include (but are not limited to) ortho-hydroxy atorvastatin and para-hydroxy atorvastatin. As used herein, the term “ortho-hydroxy atorvastatin” (or o-hydroxy atorvastatin) refers to compound (3R,5R)-7-[2-(4-fluorophenyl)-4-[(2-hydroxyphenyl)carbamoyl]-3-phenyl-5-propan-2-ylpyrrol-1-yl]-3,5-dihydroxyheptanoic acid. The term “para-hydroxy atorvastatin” (or p-hydroxy atorvastatin) refers to compound (3R,5R)-7-[2-(4-fluorophenyl)-4-[(4-hydroxyphenyl)carbamoyl]-3-phenyl-5-propan-2-ylpyrrol-1-yl]-3,5-dihydroxyheptanoic acid.

Metabolites of simvastatin include (but are not limited to) the 3-hydroxyacid metabolite. As used herein, the term “β-hydroxysimvastatin acid” refers to compound (3R,5R)-7-[(1S,2S,6R,8S,8aR)-8-(2,2-dimethylbutanoyloxy)-2,6-dimethyl-1,2,6,7,8,8a-hexahydronaphthalen-1-yl]-3,5-dihydroxyheptanoic acid. Other metabolites include 6′-beta-hydroxysimvastatin ((1S,3S,7S,8S,8aR)-3-hydroxy-8-{2-[(2R,4R)-4-hydroxy-6-oxooxan-2-yl]ethyl}-3,7-dimethyl-1,2,3,7,8,8a-hexahydronaphthalen-1-yl 2,2-dimethylbutanoate), 6′-hydroxymethyl simvastatin ((1S,3R,7R,8S,8aR)-4,6-dihydroxy-8-{2-[(2R,4R)-4-hydroxy-6-oxooxan-2-yl]ethyl}-3,7-dimethyl-1,2,3,4,6,7,8,8a-octahydronaphthalen-1-yl 2,2-dimethylbutanoate), and 6′-exomethylene simvastatin ((1S,7S,8S,8aR)-8-{2-[(2R,4R)-4-hydroxy-6-oxooxan-2-yl]ethyl}-7-methyl-3-methylidene-1,2,3,7,8,8a-hexahydronaphthalen-1-yl 2,2-dimethylbutanoate).

Further abbreviations and acronyms are given in the table below.

| | |
|---|---|
| ACTH | adrenocorticotropic hormone |
| AE | adverse event |
| ALT | alanine aminotransferase (also known as serum glutamic- |
| | pyruvic transaminase (SGPT)) |
| ANOVA | analysis of variance |
| AST | aspartate aminotransferase (also known as serum |
| | glutamic oxaloacetic transaminase (SGOT)) |
| BCRP | breast cancer resistance protein |
| BUN | blood urea nitrogen |
| C | Celsius |
| CBC | complete blood count |
| CHF | congestive heart failure |
| CKD | chronic kidney disease |
| CKD-EPI | Chronic Kidney Disease Epidemiology Collaboration |
| CMH | Cochran-Mantel-Haenszel |
| CPK | creatine phosphokinase |
| CRF | case report form |
| CRO | contract research organization |
| CRU | clinical research unit |
| CS | clinically significant |
| CV | cardiovascular |
| CVD | cardiovascular disease |
| dL | deciliter |
| DVT | deep venous thrombosis |
| EAC | Endpoint Adjudication Committee |
| ECG | electrocardiogram |
| EDC | electronic data capture |
| eGFR | estimated glomerular filtration rate |
| EOT | end of treatment |
| EPO | erythropoietin |
| ESA | erythropoiesis-stimulating agent |
| ESRD | end-stage renal disease |
| EU | European Union |
| F | Fahrenheit |
| FDA | Food and Drug Administration |
| g | gram |
| GCP | Good Clinical Practice |
| GFR | glomerular filtration rate |
| GMP | Good Manufacturing Practice |
| HA | health authority |
| HDL | high-density lipoprotein |
| Hb | Hemoglobin |
| Hgb | hemoglobin |
| HIF | hypoxia-inducible factor |
| HIFPH | hypoxia-inducible factor prolyl-hydroxylase |
| HIF-PHI | hypoxia-inducible factor prolyl-hydroxylase inhibitor |
| IC₅₀ | 50% inhibitory concentration |
| ICH | International Conference on Harmonization |
| IDMC | Independent Data Monitoring Committee |
| IDMS | isotope dilution mass spectrometry |
| IEC | independent ethics committee |
| INR | international normalized ratio |
| IRB | institutional review board |
| IV | intravenous(ly) |
| IWR | interactive web response |
| JSDT | Japanese Society for Dialysis Therapy |
| JSN | Japanese Society of Nephrology |
| KDIGO | Kidney Disease: Improving Global Outcomes |
| kg | kilogram |
| LDH | lactate dehydrogenase |
| LDL | low-density lipoprotein |
| LLN | lower limit of normal |
| MACE | major adverse cardiovascular events |
| MCH | mean corpuscular (cell) hemoglobin |
| MCHC | mean corpuscular (cell) hemoglobin concentration |
| MCV | mean corpuscular (cell) volume |
| MedDRA | Medical Dictionary for Regulatory Activities |
| μM | micromolar |
| mg | milligram |
| mL | milliliter |
| mRNA | messenger ribonucleic acid |
| MTD | maximum tolerated dose |
| NDD-CKD | non-dialysis dependent chronic kidney disease |
| ng | nanogram |
| OAT | organic anion transporter |
| PD | pharmacodynamics(s) |
| PE | pulmonary embolism |
| PHD | prolyl 4-hydroxylase domain |
| PK | pharmacokinetic(s) |
| PP | per protocol |
| PT | prothrombin time |
| PTT | partial thromboplastin time |
| QA | quality assurance |
| QC | quality control |
| QD | once daily |
| RBC | red blood cell |
| RDW | red cell distribution width |
| ROW | rest of world |
| SAE | serious adverse event |
| SAP | Statistical Analysis Plan |
| SC | subcutaneous(ly) |
| SGOT | serum glutamic oxaloacetic transaminase (also known as |
| | aspartate aminotransferase (AST)) |
| SGPT | serum glutamic pyruvic transaminase (also known as |
| | alanine aminotransferase (ALT)) |
| SmPC | summary of product characteristics |
| SV | Screening visit |
| TEAE | treatment-emergent adverse event |
| TIBC | total iron binding capacity |
| TREAT | Trial to Reduce Cardiovascular Events with Aranesp |
| | Therapy |
| TSAT | transferrin saturation |
| uACR | urine albumin-to-creatinine ratio |
| ULN | upper limit of normal |
| US | United States |
| VEGF | vascular endothelial growth factor |
| WBC | white blood cell |
| WHO | World Health Organization |

Methods of the Invention

Methods described herein can modulate a drug-drug interaction between one drug (e.g., a first drug) and another drug (e.g., a second drug) in a subject having renal anemia (anemia secondary to or associated with chronic kidney disease), where one drug (e.g., the first drug) is a HIF-PH inhibitor (e.g., {[5-(3-chlorophenyl)-3-hydroxypyridine-2-carbonyl]amino}acetic acid (Compound 1)), and the other drug (e.g., the second drug) includes but is not limited to, a drug comprising multivalent cations (e.g., an oral composition comprising calcium, iron, magnesium, lanthanum, aluminum, and the like), a statin drug (e.g., simvastatin, rosuvastatin, or atorvastatin), sulfasalazine, and furosemide.

Patient Populations/Co-Morbidities

In one aspect, methods described herein can be particularly effective for a subject having a condition (e.g., a disease or disorder) that is co-morbid to renal anemia (anemia secondary to or associated with chronic kidney disease). In embodiments, such a condition (e.g., a disease or disorder) is a complication of (e.g., induced by) renal anemia (anemia secondary to or associated with chronic kidney disease). In embodiments, such a condition (e.g., a disease or disorder) is independent of renal anemia (anemia secondary to or associated with chronic kidney disease).

In another aspect, methods described herein are particularly effective for preventing a subject having renal anemia (anemia secondary to or associated with chronic kidney disease) from developing a further medical condition (e.g., a disease or disorder). In embodiments, such a condition (e.g., a disease or disorder) is a complication of (e.g., induced by) renal anemia (anemia secondary to or associated with chronic kidney disease). In embodiments, such a condition (e.g., a disease or disorder) is independent of renal anemia (anemia secondary to or associated with chronic kidney disease).

In embodiments, a subject has or is at risk for hepatic impairment, cardiovascular disease, diabetes, dyslipidemia, elevated cholesterol or elevated triglycerides (hypertriglyceridemia) levels, thrombosis, an inflammatory gastrointestinal disorder (e.g., ulcerative colitis or Crohn's disease), rheumatoid arthritis, edema (including edema induced by chronic kidney disease, cardiovascular disease, or liver disease), and/or liver disease.

In embodiments, a subject has or is at risk for cardiovascular disease. In embodiments, a subject has or is at risk for diabetes. In embodiments, a subject has or is at risk for dyslipidemia. In embodiments, a subject has or is at risk for elevated cholesterol or elevated triglycerides (hypertriglyceridemia) levels. In embodiments, a subject has or is at risk for thrombosis. In embodiments, a subject has or is at risk for an inflammatory gastrointestinal disorder (e.g., ulcerative colitis or Crohn's disease). In embodiments, a subject has or is at risk for rheumatoid arthritis. In embodiments, a subject has or is at risk for edema (including edema induced by chronic kidney disease, cardiovascular disease, or liver disease). In embodiments, a subject has or is at risk for liver disease. In embodiments, a subject has or is at risk for hepatic impairment.

In embodiments, a subject has or is at risk for sleep disorders, somnolence, retinal hemorrhage, rotational vertigo, high blood pressure, palpitations, diarrhea, nausea, abdominal discomfort, vomiting, soft stool, gastroenteritis, stomatitis, liver dysfunction, AST elevation, rash, pruritus, eczema, erythema, alopecia, cold sweats, frequent urination, serum ferritin reduction, trans ferritin saturation reduction, fatigue, chest pain, bilirubin rise, and/or ALT rise.

Administration of Vadadustat in Subjects Receiving a Second Drug

As described above, methods described herein can be particularly effective for a subject having a condition (e.g., a disease or disorder) that is co-morbid to renal anemia (anemia secondary to or associated with chronic kidney disease), and effective for preventing a subject having renal anemia (anemia secondary to or associated with chronic kidney disease) from developing a further medical condition (e.g., a disease or disorder). Accordingly, Compound 1 may be administered to a subject receiving another drug.

In embodiments, methods described herein are effective for a subject receiving a drug that comprises a multivalent cation (e.g., a composition comprising calcium, iron, magnesium, lanthanum, aluminum, and the like), and provided herein is a method of treating renal anemia (anemia secondary to or associated with chronic kidney disease) in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving a drug that comprises a multivalent cation (e.g., a composition comprising calcium, iron, magnesium, lanthanum, aluminum, and the like).

In embodiments, a drug comprising a multivalent cation is a calcium-containing composition, an iron-containing composition, a magnesium-containing composition, a lanthanum-containing composition, or an aluminum-containing composition. In embodiments, a drug comprising a multivalent cation is an iron-containing composition (e.g., a composition comprising ferrous sulfate, sodium ferrous citrate, ferric citrate hydrate, or sucroferric oxyhydroxide). In embodiments, a drug comprising a multivalent cation is a calcium-containing composition (e.g., a composition comprising calcium acetate). In embodiments, a drug comprising a multivalent cation is a lanthanum-containing composition (e.g., a composition comprising lanthanum carbonate).

In embodiments, methods described herein are effective for a subject receiving a drug that is a Breast Cancer Resistance Protein (BCRP) substrate, and provided herein is a method of treating renal anemia (anemia secondary to or associated with chronic kidney disease) in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving a drug that is a Breast Cancer Resistance Protein (BCRP) substrate.

In embodiments, a BCRP substrate is mitoxantrone, imatinib, irinotecan, lapatinib, apixaban, atorvastatin, baricitinib, copanlisib, dolutegravir, eltrombopag, ethinylertradiol, glecaprevir, glyburide, letermovir, methotrexate, paritaprevir, pibrentasvir, pravastatin, presatovir, prucalopride, rosuvastatin, simvastatin, sofosbuvir, sulfasalazine, tenofovir, topotecan, velpatasvir, venetoclax, or voxilaprevir. In embodiments, a BCRP substrate is atorvastatin, pravastatin, rosuvastatin, simvastatin, or sulfasalazine. In embodiments, a BCRP substrate is atorvastatin, pravastatin, or sulfasalazine.

In embodiments, methods described herein are effective for a subject receiving a diuretic drug that is furosemide, and provided herein is a method of treating renal anemia (anemia secondary to or associated with chronic kidney disease) in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving furosemide.

In embodiments, methods described herein are effective for a subject receiving a statin drug, and provided herein is a method of treating renal anemia (anemia secondary to or associated with chronic kidney disease) in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving a statin drug.

In embodiments, a statin drug is simvastatin, pitavastatin, fluvastatin, lovastatin, pravastatin, rosuvastatin, or atorvastatin. In embodiments, a statin drug is simvastatin, pravastatin, rosuvastatin, or atorvastatin. In embodiments, a statin drug is pravastatin or atorvastatin.

In embodiments, methods described herein are effective for a subject receiving sulfasalazine, and provided herein is a method of treating renal anemia (anemia secondary to or associated with chronic kidney disease) in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving sulfasalazine.

In embodiments, a subject has renal anemia (anemia secondary to or associated with chronic kidney disease). In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid. In embodiments, the daily dose of {[5-(3-chlorophenyl)-3-hydroxypyridine-2-carbonyl]amino}acetic acid is increased. In embodiments, the subject is administered an initial daily dose of about 300 mg of {[5-(3-chlorophenyl)-3-hydroxypyridine-2-carbonyl]amino}acetic acid. In embodiments, the subject is administered an initial daily dose of about 450 mg of {[5-(3-chlorophenyl)-3-hydroxypyridine-2-carbonyl]amino}acetic acid. In embodiments, the subject is administered an initial daily dose of about 600 mg of {[5-(3-chlorophenyl)-3-hydroxypyridine-2-carbonyl]amino}acetic acid.

Administration of Vadadustat in Subjects with Hepatic Impairment

As described herein, therapeutic methods for patients having anemia (e.g., anemia as described herein, such as renal anemia (anemia secondary to or associated with chronic kidney disease)) comprising administration of a HIF-PH inhibitor (e.g., vadadustat) can also comprise the administration of one or more additional therapeutic agents. Such additional therapeutic agents can be useful for treating one or more co-morbid conditions (e.g., liver disease), such as those pre-existing at the time of commencement of the anemia therapy and/or arising during the period of anemia treatment. For example, Vadadustat, an oral medication developed for the treatment of anemia of chronic kidney disease (e.g., renal anemia (anemia secondary to or associated with chronic kidney disease)) in adult patients, may also be administered to patients with hepatic impairment.

In particular, hepatic impairment may alter the pharmacodynamics and/or pharmacokinetics of a drug, as well as resulting in drug accumulation or preventing active metabolite formation. Accordingly, hepatic impairment may impact systemic exposure of a drug (e.g., Vadadustat).

In embodiments, the hepatic impairment is chronic.

In embodiments, the hepatic impairment is acute.

In embodiment, hepatic impairment is induced by a hepatocellular disease.

In embodiments, hepatic impairment is induced by cholestatic disease.

In embodiments, hepatic impairment is induced by both hepatocellular disease and cholestatic disease.

In embodiments, hepatic impairment is drug-induced.

In embodiments, hepatic impairment is characterized by liver injury, liver inflammation, or liver necrosis.

In embodiments, a subject has liver fibrosis.

In embodiments, a subject has edema induced by liver disease.

In embodiments, a subject has nonalcoholic fatty liver disease (NAFLD).

In embodiments, a subject has hepatitis. In embodiments, a subject has hepatitis B.

In embodiments, a subject has hepatitis C.

Child-Pugh score (or the Child-Turcotte-Pugh score or Child Criteria) may be used to assess the prognosis of chronic liver disease, mainly cirrhosis. It breaks down patients into three categories: A—good hepatic function, B—moderately impaired hepatic function, and C
advanced hepatic dysfunction. The scoring system uses five clinical and laboratory criteria as described below to categorize patients, including variable points for each criterion based on increasing severity.
Encephalopathy: None=1 point, Grade 1 and 2=2 points, Grade 3 and 4=3 points;
Ascites: None=1 point, slight=2 points, moderate=3 points;
Bilirubin: under 2 mg/ml=1 point, 2 to 3 mg/ml=2 points, over 3 mg/ml=3 points;
Albumin: greater than 3.5 mg/ml=1 point, 2.8 to 3.5 mg/ml=2 points, less than 2.8 mg/ml=3 points;
Prothrombin Time* (sec prolonged): less than 4 sec=1 point, 4 to 6 sec=2 points, over 6 sec=3 points;
*Frequently INR may be used as a substitute for PT (Prothrombin Time), with INR under 1.7=1 point, INR 1.7 to 2.2=2 points, INR above 2.2=3 points.

Accordingly, the severity of cirrhosis may be characterized as follow:
Child Pugh Class A: 5 to 6 points;
Child Pugh Class B: 7 to 9 points;
Child Pugh Class C: 10 to 15 points;

Study has shown that vadadustat is primarily eliminated by metabolism through organs of excretion (liver and kidney). In particular, Vadadustat is primarily metabolized to O-glucuronide by uridine diphosphate (UDP)-glucuronosyltransferases (UGTs). The predominant UGT involved in the metabolism of vadadustat is UGT1A9, which is expressed in the liver and kidney.

Provided herein are methods for treating renal anemia (anemia secondary to or associated with chronic kidney disease) in a subject with hepatic impairment, comprising administering to the subject an effective amount of {[5-(3-chlorophenyl)-3-hydroxypyridine-2-carbonyl]amino}acetic acid, or a pharmaceutically acceptable salt thereof.

In embodiments, the hepatic impairment is mild or moderate hepatic impairment, or characterized as Child Pugh Class B (7-9 points).

In embodiments, the hepatic impairment is characterized by elevated bilirubin levels, decreased serum albumin levels, elevated international normalized ratios (INR), ascites, and/or hepatic encephalopathy. In embodiments, the elevated international normalized ratio (INR) is >2.20. In embodiments, the hepatic impairment is characterized by elevated bilirubin levels. In embodiments, the hepatic impairment is characterized by decreased serum albumin levels. In embodiments, the hepatic impairment is characterized by elevated international normalized ratios (INR). In embodiments, the hepatic impairment is characterized by ascites. In embodiments, the hepatic impairment is characterized by hepatic encephalopathy. In embodiments, the elevated international normalized ratio (INR) is >2.20.

In embodiments, a subject has renal anemia (anemia secondary to or associated with chronic kidney disease). In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid. In embodiments, the daily dose of {[5-(3-chlorophenyl)-3-hydroxypyridine-2-carbonyl]amino}acetic acid is increased. In embodiments, the subject is administered an initial daily dose of about 300 mg of {[5-(3-chlorophenyl)-3-hydroxypyridine-2-carbonyl]amino}acetic acid. In embodiments, the subject is administered an initial daily dose of about 450 mg of {[5-(3-chlorophenyl)-3-hydroxypyridine-2-carbonyl]amino}acetic acid.

Administration of Additional Therapeutic Agents

Therapeutic methods for patients having anemia (e.g., renal anemia (anemia secondary to or associated with chronic kidney disease)) comprising administration of a HIF-PH inhibitor (e.g., vadadustat) can also comprise the administration of one or more additional therapeutic agents. Such additional therapeutic agents can be useful for treating one or more co-morbid conditions, such as those pre-existing at the time of commencement of the anemia therapy and/or arising during the period of anemia treatment.

For example, a subject may receive an additional therapeutic agent in order to treat or prevent a co-morbid conditions such as hepatic impairment, cardiovascular disease, diabetes, dyslipidemia, elevated cholesterol or elevated triglycerides (hypertriglyceridemia) levels, thrombosis, an inflammatory gastrointestinal disorder (e.g., ulcerative colitis or Crohn's disease), rheumatoid arthritis, edema (including edema induced by chronic kidney disease, cardiovascular disease, or liver disease), and/or liver disease. In embodiments, a subject has or is at risk for cardiovascular disease. In embodiments, a subject has or is at risk for diabetes. In embodiments, a subject has or is at risk for dyslipidemia. In embodiments, a subject has or is at risk for elevated cholesterol or elevated triglycerides (hypertriglyceridemia) levels. In embodiments, a subject has or is at risk for thrombosis. In embodiments, a subject has or is at risk for an inflammatory gastrointestinal disorder (e.g., ulcerative colitis or Crohn's disease). In embodiments, a subject has or is at risk for rheumatoid arthritis. In embodiments, a subject has or is at risk for edema (including edema induced by chronic kidney disease, cardiovascular disease, or liver disease). In embodiments, a subject has or is at risk for liver disease. In embodiments, a subject has or is at risk for hepatic impairment.

In other embodiments, such co-morbid conditions also include, but are not limited to, thrombosis, sleep disorders, somnolence, retinal hemorrhage, rotational vertigo, high blood pressure, palpitations, diarrhea, nausea, abdominal discomfort, vomiting, soft stool, gastroenteritis, stomatitis, liver dysfunction, AST elevation, rash, pruritus, eczema, erythema, alopecia, cold sweats, frequent urination, serum ferritin reduction, trans ferritin saturation reduction, fatigue, chest pain, bilirubin rise, and/or ALT rise.

Other co-morbid conditions include, but are not limited to gout, gouty arthritis, hyperuricemia, high cholesterol, triglyceride levels, hypervolemia, edema, and/or other swelling related to, e.g., congestive heart failure, liver disease, kidney disease.

As described herein, significant challenges can arise when a patient receives multiple therapeutic agents, and the therapeutic agents (including a metabolite thereof) can interact with each other in the patient's body in a way that can adversely impact the intended therapeutic effect. As described herein, such drug-drug interactions can manifest in ways that impact pharmaceutical interactions, pharmacokinetic interactions, pharmacodynamic interactions, absorption, distribution, metabolism, and/or excretion. For example, a drug-drug interaction may adversely impact bioavailability, systemic exposure, and/or absorption of Compound 1 and/or another drug (e.g., a second drug such as drugs comprising a multivalent cation, statin drugs, sulfasalazine, or furosemide).

In embodiments, a drug-drug interaction may be interactions between the therapeutic agents administered to a subject. In embodiments, a drug-drug interaction may be interactions between the therapeutic agents administered to a subject and the metabolites thereof. In embodiments, a drug-drug interaction may be interactions between the metabolites of the therapeutic agents administered to a subject.

Methods described herein can modulate a drug-drug interaction between a drug (e.g., a first drug) and another (e.g., a second) drug (or a metabolite thereof), in a subject such as a subject having renal anemia (anemia secondary to or associated with chronic kidney disease). In embodiments, a drug (e.g., a first drug) is a HIF-PH inhibitor (e.g., {[5-(3-chlorophenyl)-3-hydroxypyridine-2-carbonyl]amino}acetic acid (Compound 1)). In embodiments, another (e.g., a second) drug is a drug comprising a multivalent cation (e.g., calcium, iron, magnesium, lanthanum, aluminum, and the like), a statin drug, sulfasalazine, or furosemide.

In embodiments, a method described herein prevents a drug-drug interaction. In embodiments, a method described herein controls a drug-drug interaction. In embodiments, a method described herein reduces a drug-drug interaction. In embodiments, a method described herein minimizes a drug-drug interaction.

In embodiments, a method described herein prevents a drug-drug interaction between a drug that is a HIF-PH inhibitor (e.g., {[5-(3-chlorophenyl)-3-hydroxypyridine-2-carbonyl]amino}acetic acid (Compound 1) and a drug that is a drug comprising a multivalent cation, a statin drug, sulfasalazine, or furosemide. In embodiments, a method described herein prevents a drug-drug interaction between a first drug that is a HIF-PH inhibitor (e.g., {[5-(3-chlorophenyl)-3-hydroxypyridine-2-carbonyl]amino}acetic acid (Compound 1) and a second drug that is a drug comprising a multivalent cation, a statin drug, sulfasalazine, or furosemide. In embodiments, a method described herein controls a drug-drug interaction. In embodiments, a method described herein reduces a drug-drug interaction. In embodiments, a method described herein minimizes a drug-drug interaction.

In embodiments, a drug-drug interaction relates to pharmaceutical interactions, pharmacokinetic interactions, pharmacodynamic interactions, absorption, distribution, metabolism, and/or excretion. In embodiments, a method described herein increases bioavailability of a drug. In embodiments, a method described herein maintains bioavailability of a drug. In embodiments, a method described herein reduces (e.g., minimizes) an increase in exposure to a drug (or a metabolite thereof). In embodiments, a method described herein prevents an increase in exposure to a drug (or a metabolite thereof). In embodiments, a method described herein controls an increase in exposure to a drug (or a metabolite thereof). In embodiments, a method described herein reduces (e.g., minimizes) a decrease in absorption of a drug. In embodiments, a method described herein prevents a decrease in absorption of a drug. In embodiments, a method described herein controls a decrease in absorption of a drug.

In embodiments, bioavailability of a drug is the bioavailability of a therapeutic agent administered to a subject. In embodiments, bioavailability of a drug is the bioavailability of a metabolite of a therapeutic agent administered to a subject.

In embodiments, exposure to a drug is the exposure to a therapeutic agent administered to a subject. In embodiments, exposure to a drug is the exposure to a metabolite of a therapeutic agent administered to a subject.

In embodiments, a drug (e.g., a second drug) is a drug comprising a multivalent cation such as calcium, iron, magnesium, lanthanum, aluminum, and the like. In embodiments, a method described herein controls drug-multivalent cation (e.g., calcium, iron, magnesium, lanthanum, or aluminum) chelate formation. In embodiments, a method described herein minimizes drug-multivalent cation (e.g., calcium, iron, magnesium, lanthanum, or aluminum) chelate formation. In embodiments, a method described herein reduces drug-multivalent cation (e.g., calcium, iron, magnesium, lanthanum, or aluminum) chelate formation. In embodiments, a method described herein prevents drug-multivalent cation (e.g., calcium, iron, magnesium, lanthanum, or aluminum) chelate formation. In embodiments, a method described herein controls drug-iron chelate formation. In embodiments, a method described herein minimizes drug-iron chelate formation. In embodiments, a method described herein reduces drug-iron chelate formation. In embodiments, a method described herein prevents drug-iron chelate formation. In embodiments, a drug-drug interaction (e.g., as described herein) is modulated by adjustment of the dosage amount of at least one therapeutic agent administered to a patient. In embodiments, the dosage amount of a drug that is a HIF-PH inhibitor (e.g., {[5-(3-chlorophenyl)-3-hydroxypyridine-2-carbonyl]amino}acetic acid (Compound 1), or a pharmaceutically acceptable salt thereof, or a pharmaceutical composition thereof) is adjusted. In embodiments, the dosage amount is increased. In embodiments, the dosage amount is decreased. In embodiments, the dosage amount of a drug (e.g., a drug comprising a multivalent cation, a statin drug, sulfasalazine, or furosemide) is adjusted. In embodiments, the dosage amount is increased. In embodiments, the dosage amount is decreased. In embodiments, the dosage amounts of both drugs (e.g., a drug that is a HIF-PH inhibitor such as {[5-(3-chlorophenyl)-3-hydroxypyridine-2-carbonyl]amino}acetic acid (Compound 1), or a pharmaceutically acceptable salt thereof, and a drug that is a drug comprising a multivalent cation, a statin drug, sulfasalazine, or furosemide) are adjusted. In embodiments, the dosage amount of a first drug that is a HIF-PH inhibitor (e.g., {[5-(3-chlorophenyl)-3-hydroxypyridine-2-carbonyl]amino}acetic acid (Compound 1), or a pharmaceutically acceptable salt thereof, or a pharmaceutical composition thereof) is adjusted. In embodiments, the dosage amount is increased. In embodiments, the dosage amount is decreased. In embodiments, the dosage amount of a second drug (e.g., is a drug comprising a multivalent cation, a statin drug, sulfasalazine, or furosemide) is adjusted. In embodiments, the dosage amount is increased. In embodiments, the dosage amount is decreased. In embodiments, the dosage amounts of both a first drug (e.g., a HIF-PH inhibitor such as {[5-(3-chlorophenyl)-3-hydroxypyridine-2-carbonyl]amino}acetic acid (Compound 1), or a pharmaceutically acceptable salt thereof) and a second drug (e.g., a drug comprising a multivalent cation, a statin drug, sulfasalazine, or furosemide) are both adjusted.

Timing Adjustments

In embodiments, a drug-drug interaction (e.g., as described herein) is modulated by adjustment of the timing of administration of each therapeutic agent administered to a patient.

In embodiments, administration of a drug (e.g., a first drug) that is a HIF-PH inhibitor (e.g., {[5-(3-chlorophenyl)-3-hydroxypyridine-2-carbonyl]amino}acetic acid (Compound 1), or a pharmaceutically acceptable salt thereof) and another (e.g., a second) drug (e.g., drugs comprising a multivalent cation such as calcium, iron, magnesium, lanthanum, aluminum, and the like, statin drugs, sulfasalazine, or furosemide) occurs sequentially. In embodiments of methods described herein, administration of a drug (e.g., a first drug) that is a HIF-PH inhibitor (e.g., {[5-(3-chlorophenyl)-3-hydroxypyridine-2-carbonyl]amino}acetic acid (Compound 1), or a pharmaceutically acceptable salt thereof) and another (e.g., a second) drug (e.g., a drug comprising multivalent cations such as an iron-containing composition that is oral iron or another therapeutic as described herein) occurs sequentially.

In embodiments, a timing adjustment occurs independently of a dose adjustment.

In embodiments, both a dose adjustment and a timing adjustment are made.

In embodiments, only a timing adjustment or only a dose adjustment is made. In embodiments, only a timing adjustment is made.

In embodiments, administration of two drugs is separated by a time period that is at least about 30 minutes or at least about 1 hour to about 6 hours, at least about 2 hours to about 6 hours, at least about 2 hours to about 4 hours, at least about 3 hours to about 6 hours, at least about 4 hours to about 6 hours, at least about 1 hour to about 12 hours, at least about 2 hours to about 12 hours, at least about 3 hours to about 12 hours, at least about 4 hours to about 12 hours, or at least about 6 hours to about 12 hours.

In embodiments, administration of a first drug and a second drug is separated by a time period that is at least about 30 minutes or at least about 1 hour to about 6 hours, at least about 2 hours to about 6 hours, at least about 2 hours to about 4 hours, at least about 3 hours to about 6 hours, at least about 4 hours to about 6 hours, at least about 1 hour to about 12 hours, at least about 2 hours to about 12 hours, at least about 3 hours to about 12 hours, at least about 4 hours to about 12 hours, or at least about 6 hours to about 12 hours.

In embodiments, administration of two drugs is separated by a time period that is at least about 1 hour, 2 hours, 3 hours, 4 hours, 5 hours, 6 hours, 7 hours, 8 hours, 9 hours, 10 hours, 11 hours, or 12 hours.

In embodiments, administration of a first drug and a second drug is separated by a time period that is at least about 1 hour, 2 hours, 3 hours, 4 hours, 5 hours, 6 hours, 7 hours, 8 hours, 9 hours, 10 hours, 11 hours, or 12 hours.

In embodiments, administration of two drugs is separated by a time period that is no more than about 12 hours or about 22 hours.

In embodiments, administration of a first drug and a second drug is separated by a time period that is no more than about 12 hours or about 24 hours.

In embodiments, administration of two drugs is separated by a time period that is no more than about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, or 22 hours.

In embodiments, administration of a first drug and a second drug is separated by a time period that is no more than about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours.

In embodiments, administration of two drugs is separated by a time period that is about 1 hour to about 6 hours, about 2 hours to about 6 hours, about 2 hours to about 4 hours, about 3 hours to about 6 hours, about 4 hours to about 6 hours, about 1 hour to about 12 hours, about 2 hours to about 12 hours, about 3 hours to about 12 hours, about 4 hours to about 12 hours, or about 6 hours to about 12 hours.

In embodiments, administration of a first drug and a second drug is separated by a time period that is about 1 hour to about 6 hours, about 2 hours to about 6 hours, about 2 hours to about 4 hours, about 3 hours to about 6 hours, about 4 hours to about 6 hours, about 1 hour to about 12 hours, about 2 hours to about 12 hours, about 3 hours to about 12 hours, about 4 hours to about 12 hours, or about 6 hours to about 12 hours.

In embodiments, administration of two drugs is separated by a time period that is at least about 1 hour, about 2 hours, about 3 hours, about 4 hours, about 5 hours, about 6 hours, about 7 hours, about 8 hours, about 9 hours, about 10 hours, about 11 hours, or about 12 hours.

In embodiments, administration of a first drug and a second drug is separated by a time period that is at least about 1 hour, about 2 hours, about 3 hours, about 4 hours, about 5 hours, about 6 hours, about 7 hours, about 8 hours, about 9 hours, about 10 hours, about 11 hours, or about 12 hours.

In embodiments, administration of two drugs is separated by a time period that is at least about 2 hours.

In embodiments, administration of a first drug and a second drug is separated by a time period that is at least about 2 hours.

In embodiments, a drug that is a HIF-PH inhibitor (e.g., {[5-(3-chlorophenyl)-3-hydroxypyridine-2-carbonyl]amino}acetic acid (Compound 1), or a pharmaceutically acceptable salt thereof) is the first drug administered to the patient. In embodiments, a drug that is a HIF-PH inhibitor (e.g., {[5-(3-chlorophenyl)-3-hydroxypyridine-2-carbonyl]amino}acetic acid (Compound 1), or a pharmaceutically acceptable salt thereof) is not the first drug administered to the patient. In embodiments, a drug that is a HIF-PH inhibitor (e.g., {[5-(3-chlorophenyl)-3-hydroxypyridine-2-carbonyl]amino}acetic acid (Compound 1), or a pharmaceutically acceptable salt thereof) is the second drug administered to the patient.

Provided herein is a method of reducing or minimizing drug-drug interaction between a drug and another drug in a subject. In embodiments, one drug is given at least 2 hours before and/or after taking the other drug. Also provided herein is method of controlling drug-drug interaction between one drug and another drug in a subject, wherein one drug is given at least about 2 hours before and/or after taking the other drug. In some embodiments, the subject is administered an effective amount of one drug or a pharmaceutical composition comprising an effective amount of the drug, wherein the drug is {[5-(3-chlorophenyl)-3-hydroxypyridine-2-carbonyl]amino}acetic acid (Compound 1); and an effective amount of another drug, wherein the other drug is a drug comprising a multivalent cation, a statin drug, sulfasalazine, or furosemide. In embodiments, the other drug is a multivalent cation-containing composition (e.g., an iron-containing composition such as oral iron). In embodiments, one drug is administered about 2 hours before administration of the other drug. In embodiments, one drug is administered about 2 hours to about 4 hours before administration of the other drug. In embodiments, one drug is administered about 2 hours to about 6 hours before administration of the other drug. In embodiments, one drug is administered about 2 hours after administration of the other drug. In embodiments, one drug is administered about 2 hours to about 4 hours after administration of the other drug. In embodiments, one drug is administered about 2 hours to about 6 hours after administration of the other drug.

In embodiments, provided herein is a method of reducing or minimizing drug-drug interaction between a first drug and a second drug in a subject. In embodiments, the first drug is given at least 2 hours before and/or after taking the second drug. Also provided herein is method of controlling drug-drug interaction between a first drug and a second drug in a subject, wherein the first drug is given at least about 2 hours before and/or after taking the second drug. In some embodiments, the subject is administered an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and an effective amount of the second drug, wherein the second drug is a drug comprising a multivalent cation, a statin drug, sulfasalazine, or furosemide. In embodiments, the second drug is a multivalent cation-containing composition (e.g., an iron-containing composition such as oral iron). In embodiments, the first drug is administered about 2 hours before administration of the second drug. In embodiments, the first drug is administered about 2 hours to about 4 hours before administration of the second drug. In embodiments, the first drug is administered about 2 hours to about 6 hours before administration of the second drug. In embodiments, the first drug is administered about 2 hours after administration of the second drug. In embodiments, the first drug is administered about 2 hours to about 4 hours after administration of the second drug. In embodiments, the first drug is administered about 2 hours to about 6 hours after administration of the second drug.

Also provided herein are methods of increasing the bioavailability of one or more therapeutic agent administered to the patient. For example, when a patient receives two different therapeutic agents, methods described herein can increase the bioavailability of one or both therapeutic agents. Accordingly, in embodiments, provided herein are methods of increasing the bioavailability of a drug, comprising administering to a subject an effective amount of one drug (e.g., an effective amount of {[5-(3-chlorophenyl)-3-hydroxypyridine-2-carbonyl]amino}acetic acid (Compound 1), or a pharmaceutically acceptable salt thereof); and an effective amount of another drug (e.g., statin drugs, sulfasalazine, or furosemide or a drug comprising a multivalent cation (e.g., an iron-containing composition such as oral iron)). In embodiments, one drug is {[5-(3-chlorophenyl)-3-hydroxypyridine-2-carbonyl]amino}acetic acid (Compound 1), or a pharmaceutically acceptable salt thereof. In embodiments, the other drug is a drug comprising a multivalent cation. In embodiments, the other drug is an iron-containing composition (e.g., oral iron). In embodiments, one drug is given at least about 2 hours before and/or after taking the other drug (e.g., an iron-containing composition). In embodiments, one drug is administered about 2 hours before administration of the other drug. In embodiments, one drug is administered about 2 hours to about 4 hours before administration of the other drug. In embodiments, one drug is administered about 2 hours to about 6 hours before administration of the other drug. In embodiments, one drug is administered about 2 hours after administration of the other drug. In embodiments, one drug is administered about 2 hours to about 4 hours after administration of the other drug. In embodiments, one drug is administered about 2 hours to 6 hours after administration of the other drug. In embodiments, bioavailability of one of the two drugs is increased. In embodiments, bioavailability of both drugs are increased.

In embodiments, provided herein are methods of increasing the bioavailability of a drug, comprising administering to a subject an effective amount of a first drug (e.g., an effective amount of {[5-(3-chlorophenyl)-3-hydroxypyridine-2-carbonyl]amino}acetic acid (Compound 1), or a pharmaceutically acceptable salt thereof); and an effective amount of a second drug that is a drug comprising a multivalent cation, a statin drug, sulfasalazine, or furosemide. In embodiments, a first drug is {[5-(3-chlorophenyl)-3-hydroxypyridine-2-carbonyl]amino}acetic acid (Compound 1), or a pharmaceutically acceptable salt thereof. In embodiments, the second drug is a drug comprising a multivalent cation (e.g., an iron-containing composition such as oral iron). In embodiments, the second drug is an iron-containing composition (e.g., oral iron). In embodiments, the first drug is given at least about 2 hours before and/or after taking the second drug (e.g., an iron-containing composition). In embodiments, the first drug is administered about 2 hours before administration of the second drug. In embodiments, the first drug is administered about 2 hours to about 4 hours before administration of the second drug. In embodiments, the first drug is administered about 2 hours to about 6 hours before administration of the second drug. In embodiments, the first drug is administered about 2 hours after administration of the second drug. In embodiments, the first drug is administered about 2 hours to about 4 hours after administration of the second drug. In embodiments, the first drug is administered about 2 hours to 6 hours after administration of the second drug. In embodiments, bioavailability of the first drug is increased. In embodiments, bioavailability of the second drug is increased. In embodiments, bioavailability of both drugs are increased.

Also provided herein are methods of maintaining bioavailability of one or more therapeutic agents administered to a patient. For example, when a patient receives two different therapeutic agents, methods described herein can maintain the bioavailability of one or both therapeutic agents. Accordingly, in embodiments, provided herein are methods for maintaining the bioavailability of a drug, comprising administering to a subject an effective amount of a drug (e.g., an effective amount of {[5-(3-chlorophenyl)-3-hydroxypyridine-2-carbonyl]amino}acetic acid (Compound 1), or a pharmaceutically acceptable salt thereof); and an effective amount of another drug that is a drug comprising a multivalent cation, a statin drug, sulfasalazine, or furosemide. In embodiments, one drug is {[5-(3-chlorophenyl)-3-hydroxypyridine-2-carbonyl]amino}acetic acid (Compound 1), or a pharmaceutically acceptable salt thereof. In embodiments, the other drug is a drug comprising a multivalent cation (e.g., an iron-containing composition). In embodiments, the other drug is an iron-containing composition (e.g., oral iron). In embodiments, one drug is given at least about 2 hours before and/or after taking the iron-containing composition. In embodiments, one drug is administered about 2 hours before administration of the other drug. In embodiments, one drug is administered about 2 hours to about 4 hours before administration of the other drug. In embodiments, one drug is administered about 2 hours to about 6 hours before administration of the other drug. In embodiments, one drug is administered about 2 hours after administration of the other drug. In embodiments, one drug is administered about 2 hours to about 4 hours after administration of the other drug. In embodiments, one drug is administered about 2 hours to about 6 hours after administration of the other drug. In embodiments, bioavailability of one of the two drugs is maintained. In embodiments, bioavailability of both drugs are maintained.

In embodiments, provided herein are methods for maintaining the bioavailability of a drug, comprising administering to a subject an effective amount of a first drug (e.g., an effective amount of {[5-(3-chlorophenyl)-3-hydroxypyridine-2-carbonyl]amino}acetic acid (Compound 1), or a pharmaceutically acceptable salt thereof); and an effective amount of a second drug that is a drug comprising a multivalent cation, a statin drug, sulfasalazine, or furosemide. In embodiments, a first drug is {[5-(3-chlorophenyl)-3-hydroxypyridine-2-carbonyl]amino}acetic acid (Compound 1), or a pharmaceutically acceptable salt thereof. In embodiments, the second drug is a drug comprising a multivalent cation (e.g., an iron-containing composition). In embodiments, the second drug is an iron-containing composition (e.g., oral iron). In embodiments, the first drug is given at least about 2 hours before and/or after taking the iron-containing composition. In embodiments, the first drug is administered about 2 hours before administration of the second drug. In embodiments, the first drug is administered about 2 hours to about 4 hours before administration of the second drug. In embodiments, the first drug is administered about 2 hours to about 6 hours before administration of the second drug. In embodiments, the first drug is administered about 2 hours after administration of the second drug. In embodiments, the first drug is administered about 2 hours to about 4 hours after administration of the second drug. In embodiments, the first drug is administered about 2 hours to about 6 hours after administration of the second drug. In embodiments, bioavailability of the first drug is maintained. In embodiments, bioavailability of the second drug is maintained. In embodiments, bioavailability of both drugs are maintained.

Also provided herein are methods of controlling a decrease in the absorption of one or more therapeutic agents administered to a patient. For example, when a patient receives two different therapeutic agents, methods described herein can control a decrease in the absorption of one or both therapeutic agents. Accordingly, in embodiments, provided herein are methods of controlling a decrease in the absorption of a drug, comprising administering to a subject an effective amount of a drug (e.g., an effective amount of {[5-(3-chlorophenyl)-3-hydroxypyridine-2-carbonyl]amino}acetic acid (Compound 1), or a pharmaceutically acceptable salt thereof); and an effective amount of another drug that is a drug comprising a multivalent cation, a statin drug, sulfasalazine, or furosemide. In embodiments, one drug is {[5-(3-chlorophenyl)-3-hydroxypyridine-2-carbonyl]amino}acetic acid (Compound 1), or a pharmaceutically acceptable salt thereof. In embodiments, the other drug is a drug comprising a multivalent cation (e.g., an iron-containing composition). In embodiments, the other drug is an iron-containing composition (e.g., oral iron). In embodiments, one drug is given at least 2 hours before and/or after taking the other drug. In embodiments, one drug is given at least about 2 hours before and/or after taking the iron-containing composition. In embodiments, one drug is administered about 2 hours before administration of the other drug. In embodiments, one drug is administered about 2 hours to about 4 hours before administration of the other drug. In embodiments, one drug is administered about 2 hours to about 6 hours before administration of the other drug. In embodiments, one drug is administered about 2 hours after administration of the other drug. In embodiments, one drug is administered about 2 hours to about 4 hours after administration of the other drug. In embodiments, one drug is administered about 2 hours to about 6 hours after administration of the other drug. In embodiments, a decrease of absorption of one of the two drugs is controlled (e.g., there is no change in absorption or the change of absorption is less than about 25%, about 20%, about 15%, about 10%, or about 5%). In embodiments, decreases of absorption of both drugs are controlled (e.g., for each drug, there is independently no change in absorption or the change of absorption is less than about 25%, about 20%, about 15%, about 10%, or about 5%).

In embodiments, provided herein are methods of controlling a decrease in the absorption of a drug, comprising administering to a subject an effective amount of a first drug (e.g., an effective amount of {[5-(3-chlorophenyl)-3-hydroxypyridine-2-carbonyl]amino}acetic acid (Compound 1), or a pharmaceutically acceptable salt thereof); and an effective amount of a second drug that is a drug comprising a multivalent cation, a statin drug, sulfasalazine, or furosemide. In embodiments, a first drug is {[5-(3-chlorophenyl)-3-hydroxypyridine-2-carbonyl]amino}acetic acid (Compound 1), or a pharmaceutically acceptable salt thereof. In embodiments, the second drug is a drug comprising a multivalent cation (e.g., an iron-containing composition). In embodiments, the second drug is an iron-containing composition (e.g., oral iron). In embodiments, the first drug is given at least 2 hours before and/or after taking the second drug. In embodiments, the first drug is given at least about 2 hours before and/or after taking the iron-containing composition. In embodiments, the first drug is administered about 2 hours before administration of the second drug. In embodiments, the first drug is administered about 2 hours to about 4 hours before administration of the second drug. In embodiments, the first drug is administered about 2 hours to about 6 hours before administration of the second drug. In embodiments, the first drug is administered about 2 hours after administration of the second drug. In embodiments, the first drug is administered about 2 hours to about 4 hours after administration of the second drug. In embodiments, the first drug is administered about 2 hours to about 6 hours after administration of the second drug. In embodiments, a decrease of absorption of the first drug is controlled (e.g., there is no change in absorption or the change of absorption is less than about 25%, about 20%, about 15%, about 10%, or about 5%). In embodiments, a decrease of absorption of the second drug is controlled (e.g., there is no change in absorption or the change of absorption is less than about 25%, about 20%, about 15%, about 10%, or about 5%). In embodiments, decreases of absorption of both drugs are controlled (e.g., for each drug, there is independently no change in absorption or the change of absorption is less than about 25%, about 20%, about 15%, about 10%, or about 5%).

Also provided herein are methods of controlling drug-cation chelate formation (e.g., drug-iron chelate formation when an iron-containing composition is administered to a patient) in patients receiving two or more therapeutic agents. In embodiments, a method comprises administering to a subject an effective amount of a drug (e.g., an effective amount of {[5-(3-chlorophenyl)-3-hydroxypyridine-2-carbonyl]amino}acetic acid (Compound 1), or a pharmaceutically acceptable salt thereof); and an effective amount of another drug comprising multivalent cations (e.g., an iron-containing composition). In embodiments, a drug is {[5-(3-chlorophenyl)-3-hydroxypyridine-2-carbonyl]amino}acetic acid (Compound 1), or a pharmaceutically acceptable salt thereof. In embodiments, the other drug is an iron-containing composition (e.g., oral iron). In embodiments, one drug is given at least 2 hours before and/or after taking the other drug (e.g., an iron-containing composition). In embodiments, one drug is given at least about 2 hours before and/or after taking the iron-containing composition. In embodiments, one drug is administered about 2 hours before administration of the other drug. In embodiments, one drug is administered about 2 hours to about 4 hours before administration of the other drug. In embodiments, one drug is administered about 2 hours to about 6 hours before administration of the other drug. In embodiments, one drug is administered about 2 hours after administration of the other drug. In embodiments, one drug is administered about 2 hours to about 4 hours after administration of the other drug. In embodiments, one drug is administered about 2 hours to about 6 hours after administration of the other drug.

In embodiments, a method comprises administering to a subject an effective amount of a first drug (e.g., an effective amount of {[5-(3-chlorophenyl)-3-hydroxypyridine-2-carbonyl]amino}acetic acid (Compound 1), or a pharmaceutically acceptable salt thereof); and an effective amount of a second drug that is a drug comprising a multivalent cation, a statin drug, sulfasalazine, or furosemide. In embodiments, a first drug is {[5-(3-chlorophenyl)-3-hydroxypyridine-2-carbonyl]amino}acetic acid (Compound 1), or a pharmaceutically acceptable salt thereof. In embodiments, the second drug is a drug comprising multivalent cations (e.g., an iron-containing composition). In embodiments, the second drug is an iron-containing composition (e.g., oral iron). In embodiments, the first drug is given at least 2 hours before and/or after taking a second drug (e.g., an iron-containing composition). In embodiments, the first drug is given at least about 2 hours before and/or after taking the iron-containing composition. In embodiments, the first drug is administered about 2 hours before administration of the second drug. In embodiments, the first drug is administered about 2 hours to about 4 hours before administration of the second drug. In embodiments, the first drug is administered about 2 hours to about 6 hours before administration of the second drug. In embodiments, the first drug is administered about 2 hours after administration of the second drug. In embodiments, the first drug is administered about 2 hours to about 4 hours after administration of the second drug. In embodiments, the first drug is administered about 2 hours to about 6 hours after administration of the second drug.

Also provided herein are methods of minimizing or reducing drug-cation chelate formation (e.g., drug-iron chelate formation when an iron-containing composition is administered to a patient). In embodiments, a method comprises administering to a subject an effective amount of a drug (e.g., an effective amount of {[5-(3-chlorophenyl)-3-hydroxypyridine-2-carbonyl]amino}acetic acid (Compound 1), or a pharmaceutically acceptable salt thereof, or a pharmaceutically acceptable composition thereof); and an effective amount of another drug comprising a multivalent cation (e.g., an iron-containing composition). In embodiments, a drug is {[5-(3-chlorophenyl)-3-hydroxypyridine-2-carbonyl]amino}acetic acid (Compound 1), or a pharmaceutically acceptable salt thereof. In embodiments, the other drug is an iron-containing composition (e.g., oral iron). In embodiments, one drug is given at least 2 hours before and/or after taking the other drug (e.g., an iron-containing composition). In embodiments, one drug is given at least about 2 hours before and/or after taking the other drug (e.g., an iron-containing composition). In embodiments, one drug is administered about 2 hours before administration of the other drug. In embodiments, one drug is administered about 2 hours to about 4 hours before administration of the other drug. In embodiments, one drug is administered about 2 hours to about 6 hours before administration of the other drug. In embodiments, one drug is administered about 2 hours after administration of the other drug. In embodiments, one drug is administered about 2 hours to about 4 hours after administration of the other drug. In embodiments, one drug is administered about 2 hours to about 6 hours after administration of the other drug. In embodiments, a method comprises administering to a subject an effective amount of a first drug (e.g., an effective amount of {[5-(3-chlorophenyl)-3-hydroxypyridine-2-carbonyl]amino}acetic acid (Compound 1), or a pharmaceutically acceptable salt thereof, or a pharmaceutically acceptable composition thereof); and an effective amount of a second drug comprising a multivalent cation (e.g., an iron-containing composition). In embodiments, a first drug is {[5-(3-chlorophenyl)-3-hydroxypyridine-2-carbonyl]amino}acetic acid (Compound 1), or a pharmaceutically acceptable salt thereof. In embodiments, the second drug is an iron-containing composition (e.g., oral iron). In embodiments, the first drug is given at least 2 hours before and/or after taking the second drug (e.g., an iron-containing composition). In embodiments, the first drug is given at least about 2 hours before and/or after taking the second drug (e.g., an iron-containing composition). In embodiments, the first drug is administered about 2 hours before administration of the second drug. In embodiments, the first drug is administered about 2 hours to about 4 hours before administration of the second drug. In embodiments, the first drug is administered about 2 hours to about 6 hours before administration of the second drug. In embodiments, the first drug is administered about 2 hours after administration of the second drug. In embodiments, the first drug is administered about 2 hours to about 4 hours after administration of the second drug. In embodiments, the first drug is administered about 2 hours to about 6 hours after administration of the second drug.

Also provided herein are methods of preventing drug-cation chelate formation (e.g., drug-iron chelate formation when an iron-containing composition is administered to a patient) comprising administering to a subject an effective amount of {[5-(3-chlorophenyl)-3-hydroxypyridine-2-carbonyl]amino}acetic acid (Compound 1), or a pharmaceutical composition comprising Compound 1; and an effective amount of another drug comprising a multivalent cation (e.g., an iron-containing composition), wherein Compound 1, or a pharmaceutical composition thereof, is given at least 2 hours before and/or after taking the second drug (e.g., an iron-containing composition). In embodiments, provided herein are methods of preventing drug-cation chelate formation (e.g., drug-iron chelate formation when an iron-containing composition is administered to a patient) comprising administering to a subject an effective amount of {[5-(3-chlorophenyl)-3-hydroxypyridine-2-carbonyl]amino}acetic acid (Compound 1), or a pharmaceutical composition comprising Compound 1; and an effective amount of second drug comprising a multivalent cation (e.g., an iron-containing composition), wherein Compound 1, or a pharmaceutical composition thereof, is given at least 2 hours before and/or after taking the other drug (e.g., an iron-containing composition). In embodiments, Compound 1, or a pharmaceutical composition thereof, is administered about 2 hours before administration of the iron-containing composition. In embodiments, Compound 1, or a pharmaceutical composition thereof, is administered about 2 hours to about 4 hours before administration of the iron-containing composition. In embodiments, Compound 1, or a pharmaceutical composition thereof, is administered about 2 hours to about 6 hours before administration of the iron-containing composition. In embodiments, Compound 1, or a pharmaceutical composition thereof, is administered about 2 hours after administration of the iron-containing composition. In embodiments, Compound 1, or a pharmaceutical composition thereof, is administered about 2 hours to about 4 hours after administration of the iron-containing composition. In embodiments, the Compound 1, or a pharmaceutical composition thereof, is administered about 2 hours to about 6 hours after administration of the iron-containing composition.

In some embodiments of the methods provided herein, administration of the iron-containing composition is associated with a medical treatment. For example, the drug comprising a multivalent cation (e.g., an iron-containing composition such as oral iron) may be administered as a supplement. In embodiments, the second drug comprising a multivalent cation (e.g., an iron-containing composition such as oral iron) may be administered as a supplement. Such supplementation may be needed to maintain normal ferritin and TSAT levels in subjects (e.g., at 100 ng/mL and 20%, respectively).

In some embodiments of the methods provided herein, Compound 1, or a pharmaceutical composition thereof, is administered at least 2 hours before the drug that is a drug comprising a multivalent cation (e.g., an iron-containing composition such as oral iron), a statin drug, sulfasalazine, or furosemide. In embodiments, Compound 1, or a pharmaceutical composition thereof, is administered at least 2 hours before the second drug that is a drug comprising a multivalent cation (e.g., an iron-containing composition such as oral iron), a statin drug, sulfasalazine, or furosemide. Alternatively, Compound 1, or a pharmaceutical composition thereof, is administered at least 2 hours after the drug that is a drug comprising a multivalent cation (e.g., an iron-containing composition), a statin drug, sulfasalazine, or furosemide. In certain embodiments, Compound 1, or a pharmaceutical composition thereof, is administered about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours before the drug that is a drug comprising a multivalent cation (e.g., an iron-containing composition), a statin drug, sulfasalazine, or furosemide. In embodiments, Compound 1, or a pharmaceutical composition thereof, is administered about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours before the drug that is a drug comprising a multivalent cation (e.g., an iron-containing composition), a statin drug, sulfasalazine, or furosemide. In other embodiments, Compound 1, or a pharmaceutical composition thereof, is administered about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours after the drug that is a drug comprising a multivalent cation (e.g., an iron-containing composition), a statin drug, sulfasalazine, or furosemide. In embodiments, Compound 1, or a pharmaceutical composition thereof, is administered about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours after the drug that is a drug comprising a multivalent cation (e.g., an iron-containing composition), a statin drug, sulfasalazine, or furosemide.

In embodiments, a drug that is a drug comprising a multivalent cation, a statin drug, sulfasalazine, or furosemide is the first administered composition and is administered prior to Compound 1: that is, a drug comprising a multivalent cation, a statin drug, sulfasalazine, or furosemide is administered at least about 2 hours prior to administration of Compound 1.

In embodiments, a drug comprising a multivalent cation (e.g., an iron-containing composition such as oral iron) is the first administered composition and is administered prior to Compound 1: that is, a drug comprising a multivalent cation such as an iron-containing composition (e.g., oral iron) is administered at least about 2 hours prior to administration of Compound 1.

Dose Adjustments

In embodiments, a drug-drug interaction (e.g., as described herein) is modulated by adjustment of the dosage amount of the therapeutic agent administered to a patient (e.g., adjusted compared to the amount when administered in monotherapy). In embodiments, a drug-drug interaction (e.g., as described herein) is modulated by adjustment of the dosage amount of at least one therapeutic agent administered to a patient.

In embodiments, a dose adjustment occurs independently of a timing adjustment.

In embodiments, both a dose adjustment and a timing adjustment are made.

In embodiments, only a dose adjustment or only a timing adjustment is made. In embodiments, only a dose adjustment is made.

In embodiments, the dosage amount of a drug (e.g., a first drug) that is a HIF-PH inhibitor (e.g., {[5-(3-chlorophenyl)-3-hydroxypyridine-2-carbonyl]amino}acetic acid (Compound 1), or a pharmaceutically acceptable salt thereof, or a pharmaceutical composition thereof) is adjusted. In embodiments, the dosage amount is increased. In embodiments, the dosage amount is decreased. In embodiments, the dosage amount of another (e.g., a second) drug (e.g., a drug comprising a multivalent cation, a statin drug, sulfasalazine, or furosemide) is adjusted. In embodiments, the dosage amount is increased. In embodiments, the dosage amount is decreased. In embodiments, the dosage amounts of both drugs (e.g., a drug that is a HIF-PH inhibitor such as {[5-(3-chlorophenyl)-3-hydroxypyridine-2-carbonyl]amino}acetic acid (Compound 1), or a pharmaceutically acceptable salt thereof, and a drug such as a drug comprising a multivalent cation, a statin drug, sulfasalazine, or furosemide) are adjusted. In embodiments, the dosage amount is increased. In embodiments, the dosage amount is decreased. In embodiments, the dosage amount of another (e.g., a second) drug this is a statin drug, sulfasalazine, or furosemide is adjusted. In embodiments, the dosage amount is increased. In embodiments, the dosage amount is decreased.

In embodiments, the dosage amount is increased. In embodiments, the dosage amount is increased by no more than about 100%, 200%, or 300%. In embodiments, the dosage amount is increased by more than about 300%. In embodiments, the dosage amount is increased by about 20%, 40%, 60%, 80%, 100%, 120%, 140%, 160%, 180%, 200%, 220%, 240%, 260%, 280%, or 300%. In embodiments, the dosage amount is increased by 0 to about 50%, about 50% to about 100%, about 100% to about 150%, about 150% to about 200%, about 200% to about 250%, or about 250% to about 300%. In embodiments, the dosage amount is decreased. In embodiments, the dosage amount is decreased by at least about 10%, 20%, 30%, 40%, 50%, 60%, 70%, 80%, or 90%, or 100%. In embodiments, the dosage amount is decreased by at least 0 to about 25%, at least about 25% to about 50%, at least about 50% to about 75%, or at least about 75% to about 100%. In embodiments, the dosage amount is decreased by about 10%, 20%, 30%, 40%, 50%, 60%, 70%, 80%, or 90%, or 100%. In embodiments, the dosage amount is decreased by 0 to about 25%, about 25% to about 50%, about 50% to about 75%, or about 75% to about 100%. In embodiments, the dosage amount is decreased by no more than about 25%, 50%, 75%, or 100%.

In embodiments, the dosage amount of at least one therapeutic agent is adjusted, wherein the adjustments is as described herein. In embodiments, the dosage amount is increased and the amount is as described herein. In embodiments, the dosage amount is decreased and the amount is as described herein.

In embodiments, the dosage amount of a drug (e.g., a first drug) that is a HIF-PH inhibitor (e.g., {[5-(3-chlorophenyl)-3-hydroxypyridine-2-carbonyl]amino}acetic acid (Compound 1), or a pharmaceutically acceptable salt thereof, or a pharmaceutical composition thereof) is adjusted, wherein the adjustments is as described herein. In embodiments, the dosage amount is increased and the amount is as described herein. In embodiments, the dosage amount is decreased and the amount is as described herein.

In embodiments, the dosage amount of another (e.g., a second) drug (e.g., a therapeutic agent such as a drug comprising a multivalent cation, a statin drugs (e.g., simvastatin, rosuvastatin, or atorvastatin), sulfasalazine, furosemide, or another as described herein) is adjusted, wherein the adjustments is as described herein. In embodiments, the dosage amount is increased and the amount is as described herein. In embodiments, the dosage amount is decreased and the amount is as described herein.

In embodiments, the dosage amount of another (e.g., a second) drug (e.g., a therapeutic agent such as drugs comprising a multivalent cation, statin drugs (e.g., simvastatin, rosuvastatin, or atorvastatin), sulfasalazine, furosemide, or another as described herein) is decreased, wherein the amount is as described herein. In certain embodiments, the dosage amount is decreased by about 20% to about 80%, or about 40% to about 60%. In embodiments, the dosage amount is decreased by about 10% to about 50%, or about 20% to about 40%. In embodiments, the dosage amount is decreased by about 50%.

In embodiments, the dosage amounts of both a drug (e.g., a first drug that is a HIF-PH inhibitor such as {[5-(3-chlorophenyl)-3-hydroxypyridine-2-carbonyl]amino}acetic acid (Compound 1), or a pharmaceutically acceptable salt thereof) and another (e.g., a second) drug (e.g., a therapeutic agent such as drugs comprising a multivalent cation, statin drugs (e.g., simvastatin, rosuvastatin, or atorvastatin), sulfasalazine, furosemide, or another as described herein) are both adjusted, wherein the adjustments is as described herein. In embodiments, the dosage amount is increased and the amount is as described herein. In embodiments, the dosage amount is decreased and the amount is as described herein.

In embodiments, a drug that is a HIF-PH inhibitor (e.g., {[5-(3-chlorophenyl)-3-hydroxypyridine-2-carbonyl]amino}acetic acid (Compound 1), or a pharmaceutically acceptable salt thereof) is the first drug administered to the patient. In embodiments, a drug that is a HIF-PH inhibitor (e.g., {[5-(3-chlorophenyl)-3-hydroxypyridine-2-carbonyl]amino}acetic acid (Compound 1), or a pharmaceutically acceptable salt thereof) is not the first drug administered to the patient. In embodiments, a drug that is a HIF-PH inhibitor (e.g., {[5-(3-chlorophenyl)-3-hydroxypyridine-2-carbonyl]amino}acetic acid (Compound 1), or a pharmaceutically acceptable salt thereof) is the second drug administered to the patient.

Provided herein is a method of reducing or minimizing drug-drug interaction between a drug (e.g., a first drug) and another (e.g., a second) drug (or a metabolite thereof) in a subject. In embodiments, the amount of the other (e.g., the second) drug (e.g. a drug comprising a multivalent cation, a statin drug (e.g., simvastatin, rosuvastatin, or atorvastatin), sulfasalazine, or furosemide) is adjusted compared to the amount when administered in the absence of the first drug or in monotherapy. Also provided herein is method of preventing and controlling drug-drug interaction between a drug (e.g., a first drug) and another (e.g., a second) drug (or a metabolite thereof) in a subject, wherein the amount of the other (e.g., the second) drug (e.g., a drug comprising a multivalent cation, a statin drug (e.g., simvastatin, rosuvastatin, or atorvastatin), sulfasalazine, or furosemide) is adjusted compared to the amount when administered in the absence of the first drug or in monotherapy. In embodiments, the subject is administered an effective amount of a drug (e.g., a first drug that is {[5-(3-chlorophenyl)-3-hydroxypyridine-2-carbonyl]amino}acetic acid (Compound 1), or a pharmaceutically acceptable salt thereof); and an effective amount of another (e.g., a second) drug (e.g., a drug comprising a multivalent cation, a statin drug (e.g., simvastatin, rosuvastatin, or atorvastatin), sulfasalazine, or furosemide).

Also provided herein are methods of maintaining bioavailability of one or more therapeutic agents (including a metabolite thereof) administered to a patient. For example, when a patient receives two different therapeutic agents, methods described herein can maintain the bioavailability of one or both therapeutic agents (including a metabolite thereof). Accordingly, in embodiments, provided herein are methods for maintaining the bioavailability of a drug (or a metabolite thereof), comprising administering to a subject an effective amount of a drug (e.g., a first drug that is {[5-(3-chlorophenyl)-3-hydroxypyridine-2-carbonyl]amino}acetic acid (Compound 1), or a pharmaceutically acceptable salt thereof); and an effective amount of another (e.g., a second) drug (e.g., a drug comprising a multivalent cation, a statin drug (e.g., simvastatin, rosuvastatin, or atorvastatin), sulfasalazine, or furosemide). In embodiments, bioavailability of one drug (e.g., the first drug, or a metabolite thereof) is maintained. In embodiments, bioavailability of another (e.g., the second) drug (or a metabolite thereof) is maintained. In embodiments, bioavailability of both drugs (or a metabolite thereof) are maintained.

Also provided herein are methods of minimizing, preventing and controlling an increase in exposure to one or more therapeutic agents (including a metabolite thereof) administered to a patient. For example, when a patient receives two different therapeutic agents, methods described herein can minimize, prevent and/or control an increase in exposure to one or both therapeutic agents (including a metabolite thereof). Accordingly, in embodiments, provided herein are methods of minimizing, preventing and controlling an increase in exposure to a drug (or a metabolite thereof), comprising administering to a subject an effective amount of a drug (e.g., a first drug that is {[5-(3-chlorophenyl)-3-hydroxypyridine-2-carbonyl]amino}acetic acid (Compound 1), or a pharmaceutically acceptable salt thereof); and an effective amount of another (e.g., a second) drug (e.g., a drug comprising a multivalent cation, a statin drug (e.g., simvastatin, rosuvastatin, or atorvastatin), sulfasalazine, or furosemide). In embodiments, an increase in exposure to one drug (e.g., the first drug, or a metabolite thereof) is controlled (e.g., there is no change in exposure or the change of exposure is less than about 25%, about 20%, about 15%, about 10%, or about 5%). In embodiments, an increase in exposure to the other (e.g., the second) drug (or a metabolite thereof) is controlled (e.g., there is no change in exposure or the change of exposure is less than about 25%, about 20%, about 15%, about 10%, or about 5%). In embodiments, an increase in exposure to both drugs (or a metabolite thereof) is controlled (e.g., there is no change in exposure or the change of exposure is less than about 25%, about 20%, about 15%, about 10%, or about 5%).

In embodiments, a drug (e.g., a first drug) is {[5-(3-chlorophenyl)-3-hydroxypyridine-2-carbonyl]amino}acetic acid (Compound 1), or a pharmaceutically acceptable salt thereof.

In embodiments, the other (e.g., a second) drug is a statin drug. In embodiments, the amount of the second drug that is a statin drug is decreased by at least about 10%, 20%, 30%, 40%, 50%, 60%, 70%, 80%, or 90%, or 100%. In embodiments, the dosage amount is decreased by at least 0 to about 25%, at least about 25% to about 50%, at least about 50% to about 75%, or at least about 75% to about 100%. In embodiments, the dosage amount is decreased by about 10%, 20%, 30%, 40%, 50%, 60%, 70%, 80%, or 90%, or 100%. In embodiments, the dosage amount is decreased by 0 to about 25%, about 25% to about 50%, about 50% to about 75%, or about 75% to about 100%. In embodiments, the dosage amount is decreased by no more than about 25%, 50%, 75%, or 100%. In embodiments, the amount of the second drug that is a statin drug is decreased by about 20% to about 80% compared to the amount when administered in the absence of the first drug or in monotherapy. In embodiments, the amount of the second drug that is a statin drug is decreased by about 40% to about 60% compared to the amount when administered in the absence of the first drug or in monotherapy. In embodiments, the amount of the second drug that is a statin drug is decreased by about 50% compared to the amount when administered in the absence of the first drug or in monotherapy. In embodiments, a statin drug is simvastatin, pitavastatin, fluvastatin, lovastatin, pravastatin, rosuvastatin, or atorvastatin. In embodiments, a statin drug is simvastatin, rosuvastatin, or atorvastatin. In embodiments, a statin drug is simvastatin or rosuvastatin.

In embodiments, the other (e.g., a second drug is an anti-inflammatory drug such as sulfasalazine. In embodiments, the amount of the second drug that is sulfasalazine is decreased by at least about 10%, 20%, 30%, 40%, 50%, 60%, 70%, 80%, or 90%, or 100%. In embodiments, the dosage amount is decreased by at least 0 to about 25%, at least about 25% to about 50%, at least about 50% to about 75%, or at least about 75% to about 100%. In embodiments, the dosage amount is decreased by about 10%, 20%, 30%, 40%, 50%, 60%, 70%, 80%, or 90%, or 100%. In embodiments, the dosage amount is decreased by 0 to about 25%, about 25% to about 50%, about 50% to about 75%, or about 75% to about 100%. In embodiments, the dosage amount is decreased by no more than about 25%, 50%, 75%, or 100%. In embodiments, the amount of the second drug that is sulfasalazine is decreased by about 10% to about 50% compared to the amount when administered in the absence of the first drug or in monotherapy. In embodiments, the amount of the second drug that is sulfasalazine is decreased by about 20% to about 40% compared to the amount when administered in the absence of the first drug or in monotherapy.

In embodiments, the other (e.g., a second) drug is a BCRP (breast cancer resistance protein) substrate (e.g., sulfasalazine). In embodiments, the amount of the second drug that is a BCRP substrate is decreased by at least about 10%, 20%, 30%, 40%, 50%, 60%, 70%, 80%, or 90%, or 100%. In embodiments, the dosage amount is decreased by at least 0 to about 25%, at least about 25% to about 50%, at least about 50% to about 75%, or at least about 75% to about 100%. In embodiments, the dosage amount is decreased by about 10%, 20%, 30%, 40%, 50%, 60%, 70%, 80%, or 90%, or 100%. In embodiments, the dosage amount is decreased by 0 to about 25%, about 25% to about 50%, about 50% to about 75%, or about 75% to about 100%. In embodiments, the dosage amount is decreased by no more than about 25%, 50%, 75%, or 100%. In embodiments, the amount of the second drug that is a BCRP substrate is decreased by about 10% to about 50% compared to the amount when administered in the absence of the first drug or in monotherapy. In embodiments, the amount of the second drug that is a BCRP substrate is decreased by about 20% to about 40% compared to the amount when administered in the absence of the first drug or in monotherapy.

In embodiments, a drug that is a BCRP substrate is mitoxantrone, imatinib, irinotecan, lapatinib, apixaban, atorvastatin, baricitinib, copanlisib, dolutegravir, eltrombopag, ethinylertradiol, glecaprevir, glyburide, letermovir, methotrexate, paritaprevir, pibrentasvir, pravastatin, presatovir, prucalopride, rosuvastatin, simvastatin, sofosbuvir, sulfasalazine, tenofovir, topotecan, velpatasvir, venetoclax, and voxilaprevir. In embodiments, a drug that is a BCRP substrate is atorvastatin, pravastatin, rosuvastatin, simvastatin, or sulfasalazine.

In embodiments, the other (e.g., a second) drug is a diuretic such as furosemide. In embodiments, the amount of the second drug that is furosemide is decreased by at least about 10%, 20%, 30%, 40%, 50%, 60%, 70%, 80%, or 90%, or 100%. In embodiments, the dosage amount is decreased by at least 0 to about 25%, at least about 25% to about 50%, at least about 50% to about 75%, or at least about 75% to about 100%. In embodiments, the dosage amount is decreased by about 10%, 20%, 30%, 40%, 50%, 60%, 70%, 80%, or 90%, or 100%. In embodiments, the dosage amount is decreased by 0 to about 25%, about 25% to about 50%, about 50% to about 75%, or about 75% to about 100%. In embodiments, the dosage amount is decreased by no more than about 25%, 50%, 75%, or 100%. In embodiments, the amount of the second drug that is furosemide is decreased by about 10% to about 50% compared to the amount when administered in the absence of the first drug or in monotherapy. In embodiments, the amount of the second drug that is furosemide is decreased by about 20% to about 40% compared to the amount when administered in the absence of the first drug or in monotherapy.

In some embodiments of the methods provided herein, administration of the other (e.g., a second) drug is associated with a medical treatment. In embodiments, the other (e.g., the second) drug is a statin drug that may be administered in subjects with (or at risk of) cardiovascular disease or diabetes. A statin drug may also be administered in subjects with dyslipidemia, subjects with elevated cholesterol (e.g. elevated total cholesterol, elevated LDL-cholesterol) or triglycerides (hypertriglyceridemia) levels, or subjects with low HDL-cholesterol levels. In embodiments, the other (e.g., the second) drug is sulfasalazine that may be administered in subject with (or at risk of) ulcerative colitis, Crohn's disease, or rheumatoid arthritis. In embodiments, the other (e.g., the second) drug is furosemide that may be administered in subjects with (or at risk of) edema, cardiovascular disease, or liver disease.

Methods described herein can be useful in subjects having anemia. Anemia may be characterized by hemoglobin threshold as follows:

| Age or Gender Group | Hemoglobin Threshold (g/dL) |
|---|---|
| Children | 11.0 |
| (0.50-4.99 yrs.) | |
| Children | 11.5 |
| (5.00-11.99 yrs.) | |
| Children | 12.0 |
| (12.00-14.99 yrs.) | |
| Non-pregnant Women | 12.0 |
| (≥15.00 yrs) | |
| Pregnant Women | 11.0 |
| Men (≥15.00 yrs) | 13.0 |

In embodiments, a subject has renal anemia (anemia secondary to or associated with chronic kidney disease).

Dialysis Status

The methods described herein can be beneficial to patients of different dialysis status, including the statuses described herein.

In embodiments, the patient is non-dialysis dependent. For example, in some embodiments, the patient with chronic kidney disease is non-dialysis dependent (an NDD-CKD patient).

In embodiments, the patient is dialysis-dependent. For example, in embodiments, the patient with chronic kidney disease is dialysis-dependent (a DD-CKD patient).

In embodiments, the patient receives or previously has received dialysis. In embodiments, the patient receives dialysis. In embodiments, the patient previously received dialysis.

In embodiments, dialysis is hemodialysis (HD). In embodiments, the patient with chronic kidney disease receives or previously received hemodialysis. In embodiments, the patient with chronic kidney disease receives hemodialysis. In embodiments, the patient with chronic kidney disease previously received hemodialysis.

In embodiments, dialysis is peritoneal dialysis (PD). In embodiments, the patient with chronic kidney disease receives or previously received peritoneal dialysis. In embodiments, the patient with chronic kidney disease receives peritoneal dialysis. In embodiments, the patient with chronic kidney disease previously received peritoneal dialysis.

Formulations (Pharmaceutical Compositions) of Compound 1

In certain embodiments, Compound 1 may be provided as a formulation (pharmaceutical composition). In embodiments, Compound 1 is provided as a pharmaceutical formulation that is suitable for oral administration. Such pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art.

Exemplary formulations of Compound 1 are described in WO 2014/200773 and WO 2016/161094, which are incorporated by reference in their entirety. Still further exemplary formulations are described herein.

Oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In embodiments, oral dosage forms are tablets or capsules, in which case solid excipients are employed. In another embodiment, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM. Other suitable forms of microcrystalline cellulose include, but are not limited to, silicified microcrystalline cellulose, such as the materials sold as PROSOLV 50, PROSOLV 90, PROSOLV HD90, PROSOLV 90 LM, and mixtures thereof.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is, in one embodiment, present in from about 50 to about 99 weight percent of the pharmaceutical composition or dosage form.

In embodiments, fillers may include, but are not limited to block copolymers of ethylene oxide and propylene oxide. Such block copolymers may be sold as POLOXAMER or PLURONIC, and include, but are not limited to POLOXAMER 188 NF, POLOXAMER 237 NF, POLOXAMER 338 NF, POLOXAMER 437 NF, and mixtures thereof.

In embodiments, fillers may include, but are not limited to isomalt, lactose, lactitol, mannitol, sorbitol xylitol, erythritol, and mixtures thereof.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation, and is readily discernible to those of ordinary skill in the art. In one embodiment, pharmaceutical compositions comprise from about 0.5 weight percent to about 15 weight percent of disintegrant, or from about 1 weight percent to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, povidone, crospovidone, polacrilin potassium, sodium starch glycolate, potato or tapioca starch, other starches, pre-gelatinized starch, other starches, clays, other algins, other celluloses, gums, and mixtures thereof.

Glidants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium stearyl fumarate, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional glidants include, for example, a syloid silica gel (AEROSIL200, manufactured by W. R. Grace Co. of Baltimore, Md.), a coagulated aerosol of synthetic silica (marketed by Degussa Co. of Plano, Tex.), CAB-O-SIL (a pyrogenic colloidal silicon dioxide product sold by Cabot Co. of Boston, Mass.), and mixtures thereof. If used at all, glidants may be used in an amount of less than about 1 weight percent of the pharmaceutical compositions or dosage forms into which they are incorporated.

Also provided are anhydrous pharmaceutical compositions and dosage forms since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

An anhydrous pharmaceutical composition should be prepared and stored such that its anhydrous nature is maintained. Accordingly, anhydrous compositions are, in one embodiment, packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Also provided are pharmaceutical compositions and dosage forms that comprise one or more compounds that reduce the rate by which an active ingredient will decompose. Such compounds, which are referred to herein as “stabilizers”, include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients.

In embodiments, provided herein is a tablet formulation comprising 150 mg of Compound 1. In other embodiments, provided herein is a tablet formulation comprising 300 mg of Compound 1. Exemplary 150 mg tablet and the 300 mg tablet formulations are described in Table 1.

TABLE 1
Exemplary Formulations

| | 150 mg | 300 mg |
|---|---|---|
| | Tablet | Tablet |
| Active Ingredient | {[5-(3-chlorophenyl)- | {[5-(3-chlorophenyl)- |
| | 3-hydroxypyridine-2- | 3-hydroxypyridine-2- |
| | carbonyl]amino}acetic | carbonyl]amino}acetic |
| | acid, 150 mg | acid, 300 mg |
| Additives | crystalline cellulose, | crystalline cellulose, |
| | sodium starch glycolate, | sodium starch glycolate, |
| | hypromellose, light | hypromellose, light |
| | anhydrous silicic acid, | anhydrous silicic acid, |
| | polyvinyl alcohol (partial | polyvinyl alcohol (partially |
| | saponification), macrogol | saponified), macrogol 4000, |
| | 4000, talc, titanium oxide | talc, titanium oxide, yellow |
| | | ferric oxide |

Table 2 further describes exemplary properties of each of the 150 mg tablet of Compound 1 and the 300 mg tablet of Compound 1.

TABLE 2
Properties of the 150 mg and 300 mg Tablet of Compound 1

| | 150 mg Tablet | 300 mg Tablet |
|---|---|---|
| Properties, | White film- | Yellow, oval film- |
| dosage form | coated tablet | coated tablet |
| Size (mm) | 8 | 8 (width) |
| | | 13 (length) |
| Thickness (mm) | 4 | 6 |
| Weight (mg) | 239.2 | 474.6 |

In embodiments of any method described herein, a patient receives one or more tablets of Compound 1 that is substantially according to Table 1 and/or Table 2. In embodiments, a patient receives one or more tablets of Compound 1 that is substantially according to Table 1 and/or Table 2 comprising about 150 mg Compound 1. In embodiments, a patient receives one or more tablets of Compound 1 that is substantially according to Table 1 and/or Table 2 comprising about 300 mg Compound 1. In embodiments, a patient receives a daily dose of about 150-600 mg Compound 1 (e.g., about 150, 300, 450, or 600 mg Compound 1).

Liquid Dosage Forms

Liquid dosage forms for oral administration are also provided herein. Liquid dosage forms for oral administration can include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Amounts of Compound 1 in Unit Dosage Forms

In certain other embodiments, provided herein are unit dosage forms of Compound 1 that comprise between about 150 mg and about 600 mg of a compound having a structure of Compound 1, or a pharmaceutically acceptable salt, solvate, or hydrate thereof. Such unit dosage forms can be used to provide a daily dose of Compound 1 that is about 150 mg to about 600 mg.

In certain other embodiments, provided herein are unit dosage forms of Compound 1 that comprise about 75 mg, about 150 mg, about 200 mg, about 250 mg, about 300 mg, about 350 mg, about 400 mg, about 450 mg, about 500 mg, about 550 mg, or even about 600 mg of a compound having a structure Compound 1. In certain embodiments, the unit dosage form comprises about 150 mg, about 185 mg, about 200 mg, about 250 mg, about 300 mg, or even about 315 mg of a compound having a structure of Compound 1, or a pharmaceutically acceptable salt, solvate, or hydrate thereof. In certain such embodiments, the unit dosage form is a capsule comprising about 185 mg, about 200 mg, about 200, about 250 mg, or even about 300 mg of the compound.

In embodiments, a unit dosage form comprises about 75 mg, about 150 mg, about 300 mg, about 450 mg, or about 600 mg of Compound 1. In embodiments, a unit dosage form is a tablet. In embodiments, a unit dosage form is a capsule. In embodiments, a unit dosage form comprising about 150 mg of Compound 1 is substantially the same as that exemplified in Table 1.

In embodiments, a unit dosage form comprises about 300 mg of Compound 1. In embodiments, a unit dosage form is a tablet. In embodiments, a unit dosage form is a capsule. In embodiments, a unit dosage form comprising about 300 mg of Compound 1 is substantially the same as that exemplified in Table 1.

Second Drug

In embodiments, a subject with renal anemia (anemia or secondary to chronic kidney disease) may also be administered another (e.g., a second) therapeutic agent (e.g., drugs comprising a multivalent cation, statin drugs, sulfasalazine, or furosemide) in addition to Compound 1.

It is understood that “the other”, “another”, “a second” drug or therapeutic agent as used in methods described herein also encompasses metabolites formed in vivo from an administered drug. For example, methods described herein for the modulation of a drug-drug interaction between Compound 1 and another (e.g., a second) drug can encompass modulation of a drug-drug interaction between Compound 1 and the as-administered another (e.g., a second) drug and/or Compound 1 and one or more metabolites formed in vivo from the as-administered another (e.g., a second) drug.

In embodiments, the other (e.g., a second) therapeutic agent (e.g., drugs comprising a multivalent cation, statin drugs, sulfasalazine, or furosemide) is administered simultaneously with Compound 1. In embodiments, the other (e.g., a second) therapeutic agent (e.g., drugs comprising a multivalent cation, statin drugs, sulfasalazine, or furosemide) is not administered simultaneously with Compound 1. In embodiments, the other (e.g., a second) therapeutic agent (e.g., drugs comprising a multivalent cation, statin drugs, sulfasalazine, or furosemide) is administered prior to commencement of therapy with Compound 1. In embodiments, the other (e.g., a second) therapeutic agent (e.g., drugs comprising a multivalent cation, statin drugs, sulfasalazine, or furosemide) is administered after commencing therapy with Compound 1.

In embodiments, a subject is administered another (e.g., a second) drug for treating a disease or condition in the patient that was present at the time treatment with Compound 1 was commenced.

In embodiments, a subject is administered another (e.g., a second) drug for treating or preventing a disease or condition in the patient that was not present at the time treatment with Compound 1 was commenced (e.g., the disease or condition developed after treatment with Compound 1 was commenced). In embodiments, a subject is administered another (e.g., a second) drug for treating or preventing a disease or condition in the patient induced by treatment with Compound 1. In embodiments, a subject is administered another (e.g., a second) drug for treating or preventing a disease or condition in the patient that arises independently of treatment with Compound 1.

In embodiments, a subject receives another (e.g., a second) drug (e.g., a drug comprising a multivalent cation such as iron) for treating or preventing low levels of red blood cells. In embodiments, a subject receives another (e.g., a second) drug (e.g., a drug comprising a multivalent cation such as iron) for treating or preventing low levels of healthy red blood cells due to iron deficiency. In embodiments, a subject receives another (e.g., a second) drug (e.g., a drug comprising a multivalent cation such as iron) for treating or preventing iron-deficiency anemia. In embodiments, a subject receives another (e.g., a second) drug (e.g., a drug comprising a multivalent cation such as iron) for treating or preventing low iron levels. In embodiments, a subject receives another (e.g., a second) drug (e.g., a drug comprising a multivalent cation such as iron) for treating or preventing high blood phosphate levels (hyperphosphatemia). In embodiments, a subject is on dialysis due to severe kidney disease.

In embodiments, a subject receives another (e.g., a second) drug (e.g., a drug comprising a multivalent cation such as calcium) for treating or preventing high blood phosphate levels (hyperphosphatemia). In embodiments, a subject is on dialysis due to severe kidney disease (e.g., chronic kidney disease). In embodiments, a subject receives another (e.g., a second) drug (e.g., a drug comprising a multivalent cation such as calcium) for treating or preventing impaired phosphorus excretion. In embodiments, a subject receives another (e.g., a second) drug (e.g., a drug comprising a multivalent cation such as calcium) for reducing the risk of cardiovascular disease, kidney failure, and mortality in subjects with CKD (chronic kidney disease). In embodiments, a subject receives another (e.g., a second) drug (e.g., a drug comprising a multivalent cation such as calcium) for reducing phosphorus intake and/or lowering serum phosphate levels toward the normal range. In embodiments, a subject receives another (e.g., a second) drug (e.g., a drug comprising a multivalent cation such as calcium) to maintain serum phosphorus at a target level (e.g., below 5.5 mg/dL such as about 3.5 to about 5.5 mg/dL).

In embodiments, a subject receives another (e.g., a second) drug (e.g., a drug comprising a multivalent cation such as lanthanum) for treating or preventing high blood phosphate levels (hyperphosphatemia). In embodiments, a subject is on dialysis due to severe kidney disease (e.g., chronic kidney disease). In embodiments, a subject receives another (e.g., a second) drug (e.g., a drug comprising a multivalent cation such as lanthanum) for treating or preventing impaired phosphorus excretion. In embodiments, a subject receives another (e.g., a second) drug (e.g., a drug comprising a multivalent cation such as lanthanum) for reducing the risk of cardiovascular disease, kidney failure, and mortality in subjects with CKD (chronic kidney disease). In embodiments, a subject receives another (e.g., a second) drug (e.g., a drug comprising a multivalent cation such as lanthanum) for reducing phosphorus intake and/or lowering serum phosphate levels toward the normal range. In embodiments, a subject receives another (e.g., a second) drug (e.g., a drug comprising a multivalent cation such as lanthanum) to maintain serum phosphorus at a target level (e.g., below 5.5 mg/dL such as about 3.5 to about 5.5 mg/dL).

In embodiments, a subject receives another (e.g., a second) drug (e.g., a statin drug) for treating or preventing cardiovascular disease, diabetes and/or dyslipidemia. In embodiments, a subject receives another (e.g., a second) drug (e.g., a statin drug) for treating or preventing an elevated cholesterol (e.g., total cholesterol, LDL-cholesterol) and/or triglyceride (hypertriglyceridemia) levels. In embodiments, a subject receives another (e.g., a second) drug (e.g., a statin drug) for treating or preventing heart attack, stroke, and/or acute coronary syndrome.

In embodiments, a subject receives another (e.g., a second) drug (e.g., an anti-inflammatory drug, such as, for example, sulfasalazine) for treating or preventing ulcerative colitis, Crohn's disease, or rheumatoid arthritis.

In embodiments, subject receives another (e.g., a second) drug (e.g., a diuretic, such as, for example, furosemide) for treating or preventing hypervolemia, edema (including edema induced by chronic kidney disease, cardiovascular disease, or liver disease), and/or other swelling related to, e.g., congestive heart failure, liver disease, kidney disease, and other medical conditions.

Drugs Comprising a Multivalent Cation

In embodiments, a method described herein prevents a drug-drug interaction between a drug (e.g., a first drug) that is a HIF-PH inhibitor (e.g., {[5-(3-chlorophenyl)-3-hydroxypyridine-2-carbonyl]amino}acetic acid (Compound 1) and a drug (e.g., a second drug) that comprises a multivalent cation (e.g., an iron-containing composition as described herein). In embodiments, a method described herein controls a drug-drug interaction. In embodiments, a method described herein reduces a drug-drug interaction. In embodiments, a method described herein minimizes a drug-drug interaction.

In methods described herein, a drug comprises a multivalent cation (e.g., a composition comprising calcium, iron, magnesium, lanthanum, aluminum, and the like). In embodiments, a second drug is a drug comprising a multivalent cation (e.g., a composition comprising calcium, iron, magnesium, lanthanum, aluminum, and the like).

In embodiments, a drug comprising a multivalent cation is an oral formulation. In embodiments, a second drug comprising a multivalent cation is an oral formulation.

In embodiments, a drug is a calcium-containing composition (e.g., an oral calcium supplement). In embodiments, a second drug is a calcium-containing composition (e.g., an oral calcium supplement).

In embodiments, a drug is an iron-containing composition (e.g., an oral iron supplement). In embodiments, a second drug is an iron-containing composition (e.g., an oral iron supplement).

In embodiments, a drug is a magnesium-containing composition (e.g., an oral magnesium supplement). In embodiments, a second drug is a magnesium-containing composition (e.g., an oral magnesium supplement).

In embodiments, a drug is a lanthanum-containing composition (e.g., an oral lanthanum supplement). In embodiments, a second drug is a lanthanum-containing composition (e.g., an oral lanthanum supplement).

In embodiments, a drug is an aluminum-containing composition (e.g., an oral aluminum supplement). In embodiments, a second drug is an aluminum-containing composition (e.g., an oral aluminum supplement).

In embodiments, a drug comprising a multivalent cation (e.g., a composition comprising calcium, iron, magnesium, lanthanum, aluminum, and the like) is administered prior (e.g., at least about two hours prior) to administration of Compound 1.

In embodiments, the patient has renal anemia (anemia associated with or secondary to chronic kidney disease).

In embodiments, methods described herein result in substantially no change to the AUC_{MAX} for Compound 1 (e.g., any change to the AUC_{MAX} of Compound 1 is no more than about 25%, about 20%, or about 15%).

Iron-Containing Compositions

In embodiments, a subject with renal anemia (anemia associated with or secondary to chronic kidney disease) may also be administered an iron-containing composition in addition to Compound 1. In embodiments, a subject is receiving an iron-containing composition prior to commencement of therapy with Compound 1. In embodiments, a subject receives an iron-containing composition after commencing therapy with Compound 1.

In embodiments, an iron-containing composition is formulated for oral administration (oral iron).

In embodiments, a subject is administered an iron-containing composition for treating a disease or condition in the patient that was present at the time treatment with Compound 1 was commenced.

In embodiments, a subject is administered an iron-containing composition for treating or preventing a disease or condition in the patient that was not present at the time treatment with Compound 1 was commenced (e.g., the disease or condition developed after treatment with Compound 1 was commenced). In embodiments, a subject is administered an iron-containing composition for treating or preventing a disease or condition in the patient induced by treatment with Compound 1). In embodiments, a subject is administered an iron-containing composition for treating or preventing a disease or condition in the patient that arises independently of treatment with Compound 1.

In embodiments, a subject receiving an iron-containing composition receives the iron-containing composition for treating or preventing low levels of red blood cells.

In embodiments, a subject receiving an iron-containing composition receives the iron-containing composition for treating or preventing low levels of healthy red blood cells due to iron deficiency.

In embodiments, a subject receiving an iron-containing composition receives the iron-containing composition for treating or preventing iron-deficiency anemia.

In embodiments, a subject receiving an iron-containing composition receives the iron-containing composition for treating or preventing low iron levels.

In embodiments, a subject receiving an iron-containing composition receives the iron-containing composition for treating or preventing high blood phosphate levels (hyperphosphatemia). In embodiments, a subject is on dialysis due to severe kidney disease.

In embodiments, an iron-containing composition comprises ferrous sulfate (also referred to as iron sulfate or iron (II) sulfate), ferrous citrate, ferric citrate, or sucroferric oxyhydroxide.

In embodiments, an iron-containing composition comprises ferrous citrate, ferric citrate, or sucroferric oxyhydroxide.

In embodiments, a subject receiving an iron-containing composition receives the iron-containing composition for treating or preventing hypocalcemia. In embodiments, hypocalcemia is associated with or caused by hyperphosphatemia.

In some embodiments, the iron-containing composition is an iron-containing phosphorus adsorbent. Alternatively, the iron-containing composition is an oral iron phosphorus adsorbent. Exemplary iron-containing composition include ferrous sulfate (also known as iron sulfate or iron (II) sulfate), sodium ferrous citrate, ferric citrate, or sucroferric oxyhydroxide. In embodiments, an iron-containing composition comprises sodium ferrous citrate, ferric citrate, or sucroferric oxyhydroxide.

An iron-containing composition can further comprise any of the excipients described herein (e.g., as described for formulations of Compound 1), as well as any combinations thereof.

In one embodiment, the iron-containing composition is in the form of a tablet. Such tablets may be produced by tableting, e.g., direct compressing, the iron-containing composition as a pure powder, i.e., without containing any excipient. In other embodiments, suitable excipients may be added. Such as excipients include antiadherents, binders, coatings, colors, disintegrants, flavors, glidants, lubricants, preservatives, sorbents, sweeteners, vehicles, and mixtures thereof.

In other embodiments, the tablet is obtained by compression of the granulated powders (i.e. the “inner phase”) together with further excipients (the “outer phase”). The inner phase of the iron-containing composition may comprise the phosphate adsorbent, and at least one excipient. The outer phase of the pharmaceutical composition according to the invention may comprise at least one excipient.

The pharmaceutical compositions according to the present invention may comprise a filler to provide processability.

Suitable filler materials are well-known to the art (see, e.g., Remington's Pharmaceutical Sciences, 18th Ed. (1990), Mack Publishing Co., Easton, Pa., pp. 1635-1636), and include microcrystalline cellulose, lactose and other carbohydrates, starch, pregelatinized starch, e.g., starch 1500R (Colorcon Corp.), corn starch, dicalcium phosphate, potassium bicarbonate, sodium bicarbonate, cellulose, calcium phosphate dibasic anhydrous, sugars, sodium chloride, and mixtures thereof, of which lactose, micro-crystalline cellulose, pregelatinized starch, and mixtures thereof, are preferred. Owing to its superior disintegration and compression properties, microcrystalline cellulose (Avicel grades, FMC Corp.), and mixtures comprising microcrystalline cellulose and one or more additional fillers, e.g., corn starch or pregelatinized starch, are particularly useful.

In embodiments, an iron-containing composition is formulated for oral administration.

In embodiments, an iron-containing composition is formulated for intravenous administration.

In certain embodiments, the iron-containing composition is a tablet that is formulated to be a slow-release tablet.

In other embodiments, the iron-containing composition is a tablet that is formulated to be a chewable tablet.

In certain embodiments, the iron-containing composition is administered in an amount such that ferritin is maintained at a level of between about 50 ng/mL and about 300 ng/mL.

In certain embodiments, the iron-containing composition is administered orally at a daily dose of at least about 55 mg of elemental iron.

In certain embodiments, the iron-containing composition is administered orally at a daily dose of at least about 60 mg of elemental iron.

In certain embodiments, the iron-containing composition is administered orally at a dose of about 60 mg, about 65 mg, about 70 mg, about 75 mg, about 80 mg, about 85 mg, about 90 mg, about 95 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg of elemental iron. In certain embodiments, the iron-containing composition is administered orally at a dose of about 65 mg, about 100 mg, about 200 mg, about 210 mg, about 1000 mg, or about 2000 mg of elemental iron.

In certain embodiments, the iron-containing composition is administered continuously and/or indefinitely, such as for more than 42 consecutive days. In certain alternative embodiments, the iron-containing composition is administered on an as needed basis such that ferritin is maintained at a level of between about 50 ng/mL and about 300 ng/mL.

In embodiments, an iron-containing composition comprises one or more iron (II) salts (ferrous salts). In embodiments, the iron-containing composition is formulated for oral administration.

In embodiments, an iron-containing composition comprises one or more iron (III) salts (ferric salts). In embodiments, the iron-containing composition is formulated for oral administration.

In embodiments, an iron-containing composition comprises ferrous sulfate (also known as iron sulfate or iron (II) sulfate), sodium ferrous citrate, ferric citrate, or sucroferric oxyhydroxide.

In embodiments, an iron-containing composition comprises sodium ferrous citrate, ferric citrate, or sucroferric oxyhydroxide.

Ferrous Sulfate

In embodiments, an iron-containing composition comprises ferrous sulfate (also known as iron sulfate or iron (II) sulfate).

In embodiments, a subject receives ferrous sulfate for treating or preventing low blood levels of iron. In embodiments, a subject who receives ferrous sulfate is at risk of low blood levels of iron. In embodiments, a subject receives ferrous sulfate in a dose equivalent to about 40-300 mg elemental iron. In embodiments, a subject receives ferrous sulfate in a dose equivalent to about 40-100 mg elemental iron (e.g., about 45 mg elemental iron or about 65 mg elemental iron). In embodiments, a subject receives ferrous sulfate in a dose equivalent to about 200-300 mg elemental iron (e.g., about 210 mg elemental iron). In embodiments, a subject receives ferrous sulfate as an oral solution (e.g., as a dose equivalent to about 40-100 mg elemental iron such as about 45 or about 60 mg elemental iron). In embodiments, a subject receives ferrous sulfate as oral liquid drops (e.g., as a dose equivalent to about 50-100 mg elemental iron such as about 75 mg elemental iron). In embodiments, a subject receives ferrous sulfate as a tablet (e.g., as a dose equivalent to about 40-100 mg elemental iron such as about 45 mg, about 50 mg, about 60 mg, or about 65 mg elemental iron or as a dose equivalent to about 200 to about 300 mg elemental iron such as about 215 mg elemental iron). In embodiments, a subject receives ferrous sulfate as an extended or delayed release formulation (e.g., as a tablet). In embodiments, a subject receives Compound 1 as a dose of about 150 mg. In embodiments, a subject receives Compound 1 as a dose of about 300 mg. In embodiments, a subject receives Compound 1 as a dose of about 450 mg. In embodiments, a subject receives Compound 1 as a dose of about 600 mg.

Sodium Ferrous Citrate or Ferric Citrate Hydrate

In embodiments, an iron-containing composition comprises sodium ferrous citrate. In embodiments, a subject receives sodium ferrous citrate for treating or preventing low blood levels of iron. In embodiments, a subject who receives sodium ferrous citrate is at risk of low blood levels of iron. In embodiments, a subject receives sodium ferrous citrate for treating or preventing anemia. In embodiments, a subject receives sodium ferrous citrate for treating or preventing iron deficiency. In embodiments, a subject receives sodium ferrous citrate as a dose equivalent to about 150 mg to about 300 mg elemental iron (e.g., about 200 mg elemental iron). In embodiments, a subject receives Compound 1 as a dose of about 150 mg. In embodiments, a subject receives Compound 1 as a dose of about 300 mg.

In embodiments, an iron-containing composition comprises ferric citrate. In embodiments, a subject receives ferric citrate for treating or preventing hyperphosphatemia. In embodiments, a subject receives ferric citrate for lowering high blood phosphate levels (e.g., in a subject on dialysis). In embodiments, a subject receives ferric citrate for treating or preventing anemia (e.g., iron deficiency anemia or anemia associated with or secondary to chronic kidney failure). In embodiments, a subject receives a dose of about 500-4000 mg ferric citrate (e.g., about 1000 mg, about 2000 mg, about 3000 mg, or about 4000 mg ferric citrate). In embodiments, a subject receives ferric citrate in a dose equivalent to about 200 mg to about 1000 mg elemental iron (e.g., about 210 mg, about 420 mg, about 630 mg, or about 840 mg elemental iron). In embodiments, a subject receives Compound 1 as a dose of about 150 mg. In embodiments, a subject receives Compound 1 as a dose of about 300 mg. In embodiments, a subject receives Compound 1 as a dose of about 450 mg. In embodiments, a subject receives Compound 1 as a dose of about 600 mg.

Sucroferric Oxyhydroxide

In embodiments, an iron-containing composition comprises sucroferric oxyhydroxide. In embodiments, a subject receives sucroferric oxyhydride for treating or preventing hyperphosphatemia. In embodiments, a subject receives sucroferric oxyhydride for lowering high blood phosphate levels (e.g., in a subject on dialysis). In embodiments, a subject receives sucroferric oxyhydride for treating patients with chronic kidney disease. In embodiments, a subject receives sucroferric oxyhydride for treating or preventing hypocalcemia (e.g., hypocalcemia associated with or caused by hyperphosphatemia). In embodiments, a subject receives sucroferric oxyhydroxide in a dose equivalent to about 500-2000 mg elemental iron (e.g., about 500 mg, about 1000 mg, or about 1500 mg elemental iron). In embodiments, a subject receives Compound 1 as a dose of about 150 mg. In embodiments, a subject receives Compound 1 as a dose of about 300 mg. In embodiments, a subject receives Compound 1 as a dose of about 450 mg. In embodiments, a subject receives Compound 1 as a dose of about 600 mg.

Calcium-Containing Compositions

In embodiments, a subject with renal anemia (anemia associated with or secondary to chronic kidney disease) may also be administered a calcium-containing composition in addition to Compound 1. In embodiments, a subject is receiving a calcium-containing composition prior to commencement of therapy with Compound 1. In embodiments, a subject receives a calcium-containing composition after commencing therapy with Compound 1.

In embodiments, a calcium-containing composition comprises calcium acetate.

In embodiments, a calcium-containing composition comprises calcium carbonate.

In embodiments, a calcium-containing composition is formulated for oral administration (oral calcium).

In embodiments, a subject is administered a calcium-containing composition for treating a disease or condition in the patient that was present at the time treatment with Compound 1 was commenced.

In embodiments, a subject is administered a calcium-containing composition for treating or preventing a disease or condition in the patient that was not present at the time treatment with Compound 1 was commenced (e.g., the disease or condition developed after treatment with Compound 1 was commenced). In embodiments, a subject is administered a calcium-containing composition for treating or preventing a disease or condition in the patient induced by treatment with Compound 1. In embodiments, a subject is administered a calcium-containing composition for treating or preventing a disease or condition in the patient that arises independently of treatment with Compound 1.

In some embodiments, a calcium-containing composition is a calcium-containing phosphorus adsorbent. Alternatively, a calcium-containing composition is an oral calcium phosphorus adsorbent. Exemplary calcium-containing compositions include calcium acetate and calcium carbonate.

In embodiments, a subject receiving a calcium-containing composition receives said composition for treating or preventing high blood phosphate levels (hyperphosphatemia). In embodiments, a subject is on dialysis due to severe kidney disease (e.g., chronic kidney disease).

In embodiments, a subject receiving a calcium-containing composition receives said composition for treating or preventing impaired phosphorus excretion.

In embodiments, a subject receiving a calcium-containing composition receives said composition for reducing the risk of cardiovascular disease, kidney failure, and mortality in subjects with CKD (chronic kidney disease).

In embodiments, a subject receiving a calcium-containing composition receives said composition for reducing phosphorus intake and/or lowering serum phosphate levels toward the normal range.

In certain embodiments, a subject receiving a calcium-containing composition receives said composition to maintain serum phosphorus at a target level (e.g., below 5.5 mg/dL such as about 3.5 to about 5.5 mg/dL).

A calcium-containing composition can further comprise any of the excipients described herein (e.g., as described for formulations of Compound 1), as well as any combinations thereof.

In embodiments, a calcium-containing composition is in the form of a gelcap.

In embodiments, a calcium-containing composition is in the form of a tablet including a chewable tablet. Such tablets may be produced by tableting, e.g., direct compressing, the calcium-containing composition as a pure powder, i.e., without containing any excipient. In other embodiments, suitable excipients may be added. Such as excipients include antiadherents, binders, coatings, colors, disintegrants, flavors, glidants, lubricants, preservatives, sorbents, sweeteners, vehicles, and mixtures thereof.

In other embodiments, the tablet is obtained by compression of the granulated powders (i.e. the “inner phase”) together with further excipients (the “outer phase”). The inner phase of the calcium-containing composition may comprise the phosphate adsorbent, and at least one excipient. The outer phase of the pharmaceutical composition according to the invention may comprise at least one excipient.

The pharmaceutical compositions according to the present invention may comprise a filler to provide processability.

Suitable filler materials are well-known to the art (see, e.g., Remington's Pharmaceutical Sciences, 18th Ed. (1990), Mack Publishing Co., Easton, Pa., pp. 1635-1636), and include microcrystalline cellulose, lactose and other carbohydrates, starch, pregelatinized starch, e.g., starch 1500R (Colorcon Corp.), corn starch, dicalcium phosphate, potassium bicarbonate, sodium bicarbonate, cellulose, calcium phosphate dibasic anhydrous, sugars, sodium chloride, and mixtures thereof, of which lactose, micro-crystalline cellulose, pregelatinized starch, and mixtures thereof, are preferred. Owing to its superior disintegration and compression properties, microcrystalline cellulose (Avicel grades, FMC Corp.), and mixtures comprising microcrystalline cellulose and one or more additional fillers, e.g., corn starch or pregelatinized starch, are particularly useful.

In embodiments, a calcium-containing composition is formulated for oral administration.

In certain embodiments, the calcium-containing composition is administered in an amount such that serum phosphorus is maintained at a target level (e.g., below 5.5 mg/dL such as about 3.5 to about 5.5 mg/dL).

In certain embodiments, the calcium-containing composition is administered orally at a dose of about 100 mg to about 700 mg of elemental calcium. In embodiments, the calcium-containing composition is administered orally at a dose of about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 550 mg, about 600 mg, about 650 mg, or about 700 mg of elemental calcium. In certain embodiments, the calcium-containing composition is administered orally at a dose of about 340 mg of elemental calcium. In certain embodiments, the calcium-containing composition is administered orally at a dose of about 510 mg of elemental calcium. In certain embodiments, the calcium-containing composition is administered orally at a dose of about 680 mg of elemental calcium.

In certain embodiments, the calcium-containing composition is administered continuously and/or indefinitely. In certain alternative embodiments, the calcium-containing composition is administered on an as needed basis such that serum phosphorus is maintained at a target level (e.g., below 5.5 mg/dL such as about 3.5 to about 5.5 mg/dL).

In embodiments, a calcium-containing composition comprises one or more calcium (II) salts. In embodiments, the calcium-containing composition is formulated for oral administration.

Calcium Acetate

In embodiments, a calcium-containing composition comprises calcium acetate.

In embodiments, a subject receives calcium acetate for treating or preventing high blood phosphate levels (hyperphosphatemia). In embodiments, a subject is on dialysis due to severe kidney disease (e.g., chronic kidney disease). In embodiments, a subject receives calcium acetate for treating or preventing impaired phosphorus excretion. In embodiments, a subject receives calcium acetate for reducing the risk of cardiovascular disease, kidney failure, and mortality in subjects with CKD (chronic kidney disease). In embodiments, a subject receives calcium acetate for reducing phosphorus intake and/or lowering serum phosphate levels toward the normal range. In embodiments, a subject receives calcium acetate to maintain serum phosphorus at a target level (e.g., below 5.5 mg/dL such as about 3.5 to about 5.5 mg/dL).

In embodiments, a subject receives calcium acetate at a dose of about 400 mg to about 2700 mg calcium acetate. In embodiments, a subject receives calcium acetate in a dose of about 1000 mg to about 1500 mg calcium acetate. In embodiments, a subject receives calcium acetate in a dose of about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, or about 2700 mg calcium acetate. In embodiments, a subject receives calcium acetate at a dose of about 1300 mg (e.g., 1334 mg) calcium acetate.

In embodiments, a subject is administered one time per week, two times per week, or three times per week. In embodiments, a subject is administered a dose of calcium acetate (e.g., about 1300 mg, such as 1334 mg) three times per week. For example, a subject may receive calcium acetate (e.g., about 1300 mg, such as 1334 mg) on days 3, 5, and 7 of a seven-day period. In embodiments, a subject is administered a dose of Compound 1 (e.g., 150, 300, 450, or 600 mg Compound 1) daily. In embodiments, a subject is administered a dose of Compound 1 (e.g., 150, 300, 450, or 600 mg Compound 1) 3 times per week. In embodiments, a subject is administered a dose of Compound 1 (e.g., 150, 300, 450, or 600 mg Compound 1) 4 times per week (e.g., days 1, 3, 5, and 7 of a seven-day period).

In embodiments, a subject receives calcium acetate orally. In embodiments, a subject receives calcium acetate as oral liquid drops. In embodiments, a subject receives calcium acetate as an oral solution. In embodiments, a subject receives calcium acetate as a tablet. In embodiments, a subject receives calcium acetate as gelcaps. In embodiments, a subject receives Compound 1 as a dose of about 150 mg. In embodiments, a subject receives Compound 1 as a dose of about 300 mg. In embodiments, a subject receives Compound 1 as a dose of about 450 mg. In embodiments, a subject receives Compound 1 as a dose of about 600 mg.

Lanthanum-Containing Compositions

In embodiments, a subject with renal anemia (anemia associated with or secondary to chronic kidney disease) may also be administered a lanthanum-containing composition in addition to Compound 1. In embodiments, a subject is receiving a lanthanum-containing composition prior to commencement of therapy with Compound 1. In embodiments, a subject receives a lanthanum-containing composition after commencing therapy with Compound 1.

In embodiments, a lanthanum-containing composition comprises lanthanum carbonate.

In embodiments, a lanthanum-containing composition is formulated for oral administration (oral lanthanum).

In embodiments, a subject is administered a lanthanum-containing composition for treating a disease or condition in the patient that was present at the time treatment with Compound 1 was commenced.

In embodiments, a subject is administered a lanthanum-containing composition for treating or preventing a disease or condition in the patient that was not present at the time treatment with Compound 1 was commenced (e.g., the disease or condition developed after treatment with Compound 1 was commenced). In embodiments, a subject is administered a lanthanum-containing composition for treating or preventing a disease or condition in the patient induced by treatment with Compound 1. In embodiments, a subject is administered a lanthanum-containing composition for treating or preventing a disease or condition in the patient that arises independently of treatment with Compound 1.

In some embodiments, a lanthanum-containing composition is a lanthanum-containing phosphorus adsorbent. Alternatively, a lanthanum-containing composition is an oral lanthanum phosphorus adsorbent. Exemplary lanthanum-containing compositions include lanthanum carbonate.

In embodiments, a subject receiving a lanthanum-containing composition receives said composition for treating or preventing high blood phosphate levels (hyperphosphatemia). In embodiments, a subject is on dialysis due to severe kidney disease (e.g., chronic kidney disease).

In embodiments, a subject receiving a lanthanum-containing composition receives said composition for treating or preventing impaired phosphorus excretion.

In embodiments, a subject receiving a lanthanum-containing composition receives said composition for reducing the risk of cardiovascular disease, kidney failure, and mortality in subjects with CKD (chronic kidney disease).

In embodiments, a subject receiving a lanthanum-containing composition receives said composition for reducing phosphorus intake and/or lowering serum phosphate levels toward the normal range.

In certain embodiments, a subject receiving a lanthanum-containing composition receives said composition to maintain serum phosphorus at a target level (e.g., below 5.5 mg/dL such as about 3.5 to about 5.5 mg/dL).

A lanthanum-containing composition can further comprise any of the excipients described herein (e.g., as described for formulations of Compound 1), as well as any combinations thereof.

In embodiments, a lanthanum-containing composition is in the form of a gelcap.

In embodiments, a lanthanum-containing composition is in the form of oral powder.

In embodiments, a lanthanum-containing composition is in the form of a tablet including a chewable tablet. Such tablets may be produced by tableting, e.g., direct compressing, the lanthanum-containing composition as a pure powder, i.e., without containing any excipient. In other embodiments, suitable excipients may be added. Such as excipients include antiadherents, binders, coatings, colors, disintegrants, flavors, glidants, lubricants, preservatives, sorbents, sweeteners, vehicles, and mixtures thereof.

In other embodiments, the tablet is obtained by compression of the granulated powders (i.e. the “inner phase”) together with further excipients (the “outer phase”). The inner phase of the lanthanum-containing composition may comprise the phosphate adsorbent, and at least one excipient. The outer phase of the pharmaceutical composition according to the invention may comprise at least one excipient.

The pharmaceutical compositions according to the present invention may comprise a filler to provide processability.

Suitable filler materials are well-known to the art (see, e.g., Remington's Pharmaceutical Sciences, 18th Ed. (1990), Mack Publishing Co., Easton, Pa., pp. 1635-1636), and include microcrystalline cellulose, lactose and other carbohydrates, starch, pregelatinized starch, e.g., starch 1500R (Colorcon Corp.), corn starch, dicalcium phosphate, potassium bicarbonate, sodium bicarbonate, cellulose, calcium phosphate dibasic anhydrous, sugars, sodium chloride, and mixtures thereof, of which lactose, micro-crystalline cellulose, pregelatinized starch, and mixtures thereof, are preferred. Owing to its superior disintegration and compression properties, microcrystalline cellulose (Avicel grades, FMC Corp.), and mixtures comprising microcrystalline cellulose and one or more additional fillers, e.g., corn starch or pregelatinized starch, are particularly useful.

In embodiments, a lanthanum-containing composition is formulated for oral administration.

In certain embodiments, the lanthanum-containing composition is administered in an amount such that serum phosphorus is maintained at a target level (e.g., below 5.5 mg/dL such as about 3.5 to about 5.5 mg/dL).

In certain embodiments, the lanthanum-containing composition is administered orally at a dose of about 600 mg to about 2700 mg of elemental lanthanum. In embodiments, the lanthanum containing composition is administered orally at a dose of about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 2000 mg, about 2050 mg, about 2100 mg, about 2150 mg, about 2200 mg, about 2250 mg, about 2300 mg, about 2350 mg, about 24000 mg, about 2450 mg, about 2500 mg, about 2550 mg, about 2600 mg, about 2650 mg, or about 2700 mg of elemental lanthanum. In certain embodiments, the lanthanum-containing composition is administered orally at a dose of about 900 mg to about 1900 mg of elemental lanthanum.

In certain embodiments, the lanthanum-containing composition is administered continuously and/or indefinitely. In certain alternative embodiments, the lanthanum-containing composition is administered on an as needed basis such that serum phosphorus is maintained at a target level (e.g., below 5.5 mg/dL such as about 3.5 to about 5.5 mg/dL).

In embodiments, a lanthanum-containing composition comprises one or more lanthanum (III) salts. In embodiments, the lanthanum-containing composition is formulated for oral administration (e.g., tablets).

Lanthanum Carbonate

In embodiments, a lanthanum-containing composition comprises lanthanum carbonate.

In embodiments, a subject receives lanthanum carbonate for treating or preventing high blood phosphate levels (hyperphosphatemia). In embodiments, a subject is on dialysis due to severe kidney disease (e.g., chronic kidney disease). In embodiments, a subject receives lanthanum carbonate for treating or preventing impaired phosphorus excretion. In embodiments, a subject receives lanthanum carbonate for reducing the risk of cardiovascular disease, kidney failure, and mortality in subjects with CKD (chronic kidney disease). In embodiments, a subject receives lanthanum carbonate for reducing phosphorus intake and/or lowering serum phosphate levels toward the normal range. In embodiments, a subject receives lanthanum carbonate to maintain serum phosphorus at a target level (e.g., below 5.5 mg/dL such as about 3.5 to about 5.5 mg/dL).

In embodiments, a subject receives lanthanum carbonate at a dose of about 1000 mg to about 4500 mg lanthanum carbonate. In embodiments, a subject receives lanthanum carbonate in a dose of about 1500 mg to about 3000 mg lanthanum carbonate. In embodiments, a subject receives lanthanum carbonate in a dose of about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, or about 4500 mg lanthanum carbonate. In embodiments, a subject receives lanthanum carbonate at a dose of about 1500 mg to 3000 mg lanthanum carbonate.

In embodiments, a subject is administered one time per week, two times per week, or three times per week. In embodiments, a subject is administered a dose of lanthanum carbonate three times per week. For example, a subject may receive lanthanum carbonate on days 3, 5, and 7 of a seven-day period. In embodiments, a subject is administered a dose of Compound 1 (e.g., 150, 300, 450, or 600 mg Compound 1) daily. In embodiments, a subject is administered a dose of Compound 1 (e.g., 150, 300, 450, or 600 mg Compound 1) 3 times per week. In embodiments, a subject is administered a dose of Compound 1 (e.g., 150, 300, 450, or 600 mg Compound 1) 4 times per week (e.g., days 1, 3, 5, and 7 of a seven-day period).

In embodiments, a subject receives lanthanum carbonate orally. In embodiments, a subject receives lanthanum carbonate as a tablet. In embodiments, a tablet is a chewable tablet. In embodiments, a subject receives lanthanum carbonate as a powder. In embodiments, a subject receives Compound 1 as a dose of about 150 mg. In embodiments, a subject receives Compound 1 as a dose of about 300 mg. In embodiments, a subject receives Compound 1 as a dose of about 450 mg. In embodiments, a subject receives Compound 1 as a dose of about 600 mg.

BCRP Substrates

In embodiments, the other (e.g., a second) drug or therapeutic is a human breast cancer resistance protein (BCRP) substrate. Accordingly, methods described herein can be useful for modulating drug interactions between Compound 1 and a BCRP substrate and/or one or more metabolites thereof, including as described herein.

In embodiments, a drug (e.g., a second drug) that is a BCRP substrate is mitoxantrone, imatinib, irinotecan, lapatinib, apixaban, atorvastatin, baricitinib, copanlisib, dolutegravir, eltrombopag, ethinylertradiol, glecaprevir, glyburide, letermovir, methotrexate, paritaprevir, pibrentasvir, pravastatin, presatovir, prucalopride, rosuvastatin, simvastatin, sofosbuvir, sulfasalazine, tenofovir, topotecan, velpatasvir, venetoclax, or voxilaprevir.

In embodiments, a drug (e.g., a second drug) that is a BCRP substrate is atorvastatin, pravastatin, rosuvastatin, simvastatin, or sulfasalazine.

In embodiments, the dosage amount of a drug (e.g., a second drug) that is a BCRP substrate is not adjusted when co-administered with Compound 1.

In embodiments, the dosage amount of a drug (e.g., a second drug) that is a BCRP substrate is adjusted when co-administered with Compound 1. In embodiments, the dosage amount is increased. In embodiments, the dosage amount is decreased.

Still further embodiments of methods comprising treating a disease or a condition using a BCRP substrate include those described herein.

Statins

In embodiments, a drug (e.g., a second drug) is a statin drug. Accordingly, methods described herein can be useful for modulating drug interactions between Compound 1 and a statin drug and/or one or more metabolites thereof, including as described herein.

In embodiments, a subject receives a statin drug for treating or preventing cardiovascular disease, diabetes and/or dyslipidemia. In embodiments, a subject receives a statin drug for treating or preventing an elevated cholesterol (e.g., total cholesterol, LDL-cholesterol) and/or triglyceride (hypertriglyceridemia) levels. In embodiments, a subject receives a statin drug for treating or preventing heart attack, stroke, and/or acute coronary syndrome. In embodiments, a statin drug is simvastatin, pitavastatin, fluvastatin, lovastatin, pravastatin, rosuvastatin, or atorvastatin. In embodiments, a statin drug is simvastatin, rosuvastatin, or atorvastatin. In embodiments, a statin drug is simvastatin or rosuvastatin.

In embodiments, a statin drug is Rosuvastatin. In embodiments, a subject receives Rosuvastatin at a dose of about 5 mg to about 40 mg per day. In embodiments, a subject receives Rosuvastatin at a dose of about 2.5 mg to about 20 mg per day. In embodiments, a subject receives Rosuvastatin at a dose of about 2.5 mg, about 5 mg, about 7.5 mg, about 10 mg, about 12.5 mg, about 15 mg, about 17.5 mg, about 20 mg, about 25 mg, about 30 mg, about 35 mg, or about 40 mg per day. In embodiments, a subject receives Rosuvastatin at a dose of about 5 mg, about 10 mg, about 15 mg, about 20 mg, about 25 mg, about 30 mg, about 35 mg, or about 40 mg per day. In embodiments, a subject receives Rosuvastatin at a dose of about 20 mg per day. In embodiments, a daily dose is a maximum daily dose. In embodiments, a subject receives Rosuvastatin at a maximum dose of about 20 mg per day. In embodiments, a subject receives Rosuvastatin at a maximum dose of about 10 mg per day.

In embodiments, the dosage amount of a drug (e.g., a second drug) that is Rosuvastatin is not adjusted when co-administered with Compound 1. In embodiments, the dosage amount of a drug (e.g., a second drug) that is Rosuvastatin is adjusted when co-administered with Compound 1. In embodiments, the dosage amount is increased. In embodiments, the dosage amount is decreased. In embodiments, the daily dose is decreased by at least about 2.5 mg, about 5 mg, about 7.5 mg, about 10 mg, about 12.5 mg, about 15 mg, about 17.5 mg, about 20 mg, about 25 mg, about 30 mg, or about 35 mg. In embodiments, the daily dose is decreased by at least about 5 mg, about 10 mg, about 15 mg, about 20 mg, about 25 mg, about 30 mg, or about 35 mg. In embodiments, the daily dose is decreased by about 5 mg to about 15 mg, about 15 mg to about 25 mg, or about 25 mg to about 35 mg.

In embodiments, a subject receives Rosuvastatin at maximum a dose of about 5 mg to about 40 mg per day when co-administered with Compound 1. In embodiments, a subject receives Rosuvastatin at maximum a dose of about 2.5 mg to about 20 mg per day when co-administered with Compound 1. In embodiments, a subject receives Rosuvastatin at a maximum dose of about 2.5 mg, about 5 mg, about 7.5 mg, about 10 mg, about 12.5 mg, about 15 mg, about 17.5 mg, about 20 mg, about 25 mg, about 30 mg, about 35 mg, or about 40 mg per day when co-administered with Compound 1. In embodiments, a subject receives Rosuvastatin at a maximum dose of about 5 mg, about 10 mg, about 15 mg, about 20 mg, about 25 mg, about 30 mg, about 35 mg, or about 40 mg per day when co-administered with Compound 1. In embodiments, a subject receives Rosuvastatin at a maximum dose of about 10 mg per day when co-administered with Compound 1.

In embodiments, a statin is Pravastatin. In embodiments, a subject receives Pravastatin at a dose of about 10 mg to about 80 mg per day. In embodiments, a subject receives Pravastatin at a dose of about 10 mg, about 20 mg, about 30 mg, about 40 mg, about 50 mg, about 60 mg, about 70 mg, or about 80 mg per day. In embodiments, a subject receives Pravastatin at a dose of about 40 mg per day. In embodiments, a subject receives Pravastatin at a dose of about 20 mg per day. In embodiments, a daily dose is a maximum daily dose. In embodiments, a subject receives Pravastatin at a maximum dose of about 40 mg per day. In embodiments, a subject receives Pravastatin at a maximum dose of about 20 mg per day.

In embodiments, the dosage amount of a drug (e.g., a second drug) that is Pravastatin is not adjusted when co-administered with Compound 1. In embodiments, the dosage amount of a drug (e.g., a second drug) that is Pravastatin is adjusted when co-administered with Compound 1. In embodiments, the dosage amount is increased. In embodiments, the dosage amount is decreased. In embodiments, the daily dose is decreased by at least about 5 mg, about 10 mg, about 15 mg, about 20 mg, about 25 mg, about 30 mg, or about 35 mg. about 40 mg, about 45 mg, about 50 mg, about 55 mg, about 60 mg, about 65 mg, about 70 mg, about 75 mg. In embodiments, the daily dose is decreased by about 5 mg to about 15 mg, about 15 mg to about 25 mg, about 25 mg to about 35 mg, about 35 mg to about 45 mg, about 45 mg to about 55 mg, about 55 mg to about 65 mg, or about 65 mg to about 75 mg.

In embodiments, a subject receives Pravastatin at a maximum dose of about 10 mg to about 80 mg per day when co-administered with Compound 1. In embodiments, a subject receives Pravastatin at a maximum dose of about 10 mg to about 20 mg per day when co-administered with Compound 1. In embodiments, a subject receives Pravastatin at a maximum dose of about 10 mg, about 20 mg, about 30 mg, about 40 mg, about 50 mg, about 60 mg, about 70 mg, or about 80 mg per day when co-administered with Compound 1. In embodiments, a subject receives Pravastatin at a maximum dose of about 40 mg per day.

In embodiments, a statin is Atorvastatin. For example, methods described herein can modulate a drug interaction between Compound 1 and Atorvastatin and/or one or more metabolites thereof (e.g., ortho-hydroxy Atorvastatin and/or para-hydroxy Atorvastatin). In embodiments, a method modulates a drug interaction between Compound 1 and Atorvastatin as-administered. In embodiments, a method modulates a drug interaction between Compound 1 and one or more metabolites of Atorvastatin (e.g., ortho-hydroxy Atorvastatin and/or para-hydroxy Atorvastatin). In embodiments, a method modulates a drug interaction between Compound 1 and ortho-hydroxy Atorvastatin (also known as o-hydroxy atorvastatin, or (3R,5R)-7-[2-(4-fluorophenyl)-4-[(2-hydroxyphenyl)carbamoyl]-3-phenyl-5-propan-2-ylpyrrol-1-yl]-3,5-dihydroxyheptanoic acid). In embodiments, a method modulates a drug interaction between Compound 1 and para-hydroxy Atorvastatin (also known as p-hydroxy atorvastatin, or (3R,5R)-7-[2-(4-fluorophenyl)-4-[(4-hydroxyphenyl)carbamoyl]-3-phenyl-5-propan-2-ylpyrrol-1-yl]-3,5-dihydroxyheptanoic acid).

In embodiments, a subject receives Atorvastatin at a dose of about 10 mg to about 80 mg per day. In embodiments, a subject receives Atorvastatin at a dose of about 10 mg to about 40 mg per day. In embodiments, a subject receives Atorvastatin at a dose of about 10 mg, about 15 mg, about 20 mg, about 25 mg, about 30 mg, about 35 mg, about 40 mg, about 50 mg, about 60 mg, about 70 mg, or about 80 mg per day. In embodiments, a subject receives Atorvastatin at a dose of about 10 mg, about 20 mg, about 30 mg, about 40 mg, about 50 mg, about 60 mg, about 70 mg, or about 80 mg per day. In embodiments, a subject receives Atorvastatin at a dose of about 40 mg per day. In embodiments, a daily dose is a maximum daily dose. In embodiments, a subject receives Atorvastatin at a maximum dose of about 40 mg per day.

In embodiments, the dosage amount of a drug (e.g., a second drug) that is Atorvastatin is not adjusted when co-administered with Compound 1. In embodiments, the dosage amount of a drug (e.g., a second drug) that is Atorvastatin is adjusted when co-administered with Compound 1. In embodiments, the dosage amount is increased. In embodiments, the dosage amount is decreased. In embodiments, the daily dose is decreased by at least about 5 mg, about 10 mg, about 15 mg, about 20 mg, about 25 mg, about 30 mg, or about 35 mg. about 40 mg, about 45 mg, about 50 mg, about 55 mg, about 60 mg, about 65 mg, about 70 mg, about 75 mg. In embodiments, the daily dose is decreased by about 5 mg to about 15 mg, about 15 mg to about 25 mg, about 25 mg to about 35 mg, about 35 mg to about 45 mg, about 45 mg to about 55 mg, about 55 mg to about 65 mg, or about 65 mg to about 75 mg.

In embodiments, a subject receives Atorvastatin at a maximum dose of about 10 mg to about 80 mg per day when co-administered with Compound 1. In embodiments, a subject receives Atorvastatin at a maximum dose of about 10 mg to about 40 mg per day when co-administered with Compound 1. In embodiments, a subject receives Atorvastatin at a maximum dose of about 10 mg, about 20 mg, about 30 mg, about 40 mg, about 50 mg, about 60 mg, about 70 mg, or about 80 mg per day when co-administered with Compound 1. In embodiments, a subject receives Atorvastatin at a maximum dose of about 40 mg per day when co-administered with Compound 1. In embodiments, a subject receives Atorvastatin at a maximum dose of about 20 mg to about 40 mg per day when co-administered with Compound 1.

In embodiments, a statin is Simvastatin. For example, methods described herein can modulate a drug interaction between Compound 1 and Simvastatin and/or one or more metabolites thereof (e.g., p-hydroxy Simvastatin acid, or any other metabolites as described herein). In embodiments, a method modulates a drug interaction between Compound 1 and Simvastatin as-administered. In embodiments, a method modulates a drug drug interaction between Compound 1 and one or more metabolites of Simvastatin (e.g., pi-hydroxy Simvastatin acid, or any other metabolites as described herein).

In embodiments, a subject receives Simvastatin at a dose of about 10 mg to about 80 mg per day. In embodiments, a subject receives Simvastatin at a dose of about 5 mg to about 20 mg per day. In embodiments, a subject receives Simvastatin at a dose of about 5 mg, about 10 mg, about 15 mg, about 20 mg, about 30 mg, about 40 mg, about 50 mg, about 60 mg, about 70 mg, or about 80 mg per day. In embodiments, a subject receives Simvastatin at a dose of about 10 mg, about 20 mg, about 30 mg, about 40 mg, about 50 mg, about 60 mg, about 70 mg, or about 80 mg per day. In embodiments, a subject receives Simvastatin at a dose of about 40 mg per day. In embodiments, a subject receives Simvastatin at a dose of about 20 mg per day. In embodiments, a daily dose is a maximum daily dose. In embodiments, a subject receives Simvastatin at a maximum dose of about 40 mg per day. In embodiments, a subject receives Simvastatin at a maximum dose of about 20 mg per day.

In embodiments, the dosage amount of a drug (e.g., a second drug) that is Simvastatin is not adjusted when co-administered with Compound 1. In embodiments, the dosage amount of a drug (e.g., a second drug) that is Simvastatin is adjusted when co-administered with Compound 1. In embodiments, the dosage amount is increased. In embodiments, the dosage amount is decreased. In embodiments, the daily dose is decreased by at least about 5 mg, about 10 mg, about 15 mg, about 20 mg, about 25 mg, about 30 mg, or about 35 mg. about 40 mg, about 45 mg, about 50 mg, about 55 mg, about 60 mg, about 65 mg, about 70 mg, about 75 mg. In embodiments, the daily dose is decreased by about 5 mg to about 15 mg, about 15 mg to about 25 mg, about 25 mg to about 35 mg, about 35 mg to about 45 mg, about 45 mg to about 55 mg, about 55 mg to about 65 mg, or about 65 mg to about 75 mg.

In embodiments, a subject receives Simvastatin at a maximum dose of about 10 mg to about 80 mg per day when co-administered with Compound 1. In embodiments, a subject receives Simvastatin at a maximum dose of about 5 mg to about 20 mg per day when co-administered with Compound 1. In embodiments, a subject receives Simvastatin at a maximum dose of about 5 mg, about 10 mg, about 15 mg, about 20 mg, about 30 mg, about 40 mg, about 50 mg, about 60 mg, about 70 mg, or about 80 mg per day when co-administered with Compound 1. In embodiments, a subject receives Simvastatin at a maximum dose of about 10 mg, about 20 mg, about 30 mg, about 40 mg, about 50 mg, about 60 mg, about 70 mg, or about 80 mg per day when co-administered with Compound 1. In embodiments, a subject receives Simvastatin at a maximum dose of about 20 mg per day when co-administered with Compound 1.

In certain embodiments, a statin drug as described herein is administered continuously and/or indefinitely. In certain embodiments, a statin drug is administered once, twice, or three times per day. In embodiments, a statin drug described herein is administered simultaneously with Compound 1. In embodiments, a statin drug described herein is not administered simultaneously with Compound 1.

In embodiments, a subject has renal anemia (anemia secondary to or associated with chronic kidney disease). In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid. In embodiments, the daily dose of {[5-(3-chlorophenyl)-3-hydroxypyridine-2-carbonyl]amino}acetic acid is increased. In embodiments, the subject is administered an initial daily dose of about 300 mg, 450 mg, or 600 mg of {[5-(3-chlorophenyl)-3-hydroxypyridine-2-carbonyl]amino}acetic acid.

Sulfasalazine

In embodiments, a drug (e.g., a second drug) is an anti-inflammatory drug.

In embodiments, a drug (e.g., a second drug) is sulfasalazine. Sulfasalazine is also a BCRP substrate (e.g., as described herein). For example, methods described herein can modulate a drug interaction between Compound 1 and sulfasalazine and/or one or more metabolites thereof (e.g., sulfapyridine and/or mesalamine). In embodiments, a method modulates a drug interaction between Compound 1 and sulfasalazine as-administered. In embodiments, a method modulates a drug interaction between Compound 1 and one or more metabolites of sulfasalazine (e.g., sulfapyridine and/or mesalamine). In embodiments, a method modulates a drug interaction between Compound 1 and sulfapyridine. In embodiments, a method modulates a drug interaction between Compound 1 and mesalamine.

In embodiments, a subject receives sulfasalazine for treating or preventing ulcerative colitis, Crohn's disease, or rheumatoid arthritis. In embodiments, a subject receives sulfasalazine at a dose of about 50 mg to about 1000 mg per day. In embodiments, a subject receives sulfasalazine at a dose of about 1 g to about 4 g per day. In embodiments, a subject receives sulfasalazine at a dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg per day. In embodiments, a subject receives sulfasalazine at a dose of about 1 g, about 1.1 g, about 1.2 g, about 1.3 g, about 1.4 g, about 1.5 g, about 1.6 g, about 1.7 g, about 1.8 g, about 1.9 g, about 2.0 g, about 2.1 g, about 2.2 g, about 2.3 g, about 2.4 g, about 2.5 g, about 2.6 g, about 2.7 g, about 2.8 g, about 2.9 g, about 3.0 g, about 3.1 g, 3.2 g, 3.2 g, 3.3 g, 3.4 g, 3.5 g, 3.6 g, 3.7 g, 3.8 g, 3.8 g, or 4.0 g per day. In embodiments, a subject receives sulfasalazine at a dose of about 500 mg per day. In embodiments, a daily dose is a maximum daily dose. In embodiments, a subject receives sulfasalazine at a maximum dose of about 500 mg per day.

In embodiments, the dosage amount of a drug (e.g., a second drug) that is sulfasalazine is not adjusted when co-administered with Compound 1. In embodiments, the dosage amount of a drug (e.g., a second drug) that is sulfasalazine is adjusted when co-administered with Compound 1. In embodiments, the dosage amount is increased. In embodiments, the dosage amount is decreased. In embodiments, the daily dose is decreased by at least about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, or about 950 mg. In embodiments, the daily dose is decreased by at least about 1 g, about 1.5 g, about 2 g, about 2.5 g, about 3 g, or about 3.5 g. In embodiments, the daily dose is decreased by about 50 mg to about 150 mg, about 150 mg to about 250 mg, about 250 mg to about 350 mg, about 350 mg to about 450 mg, about 450 mg to about 550 mg, about 550 mg to about 650 mg, about 650 mg to about 750 mg, about 750 mg to about 850 mg about 850 mg to about 950 mg. In embodiments, the daily dose is decreased by about 1 g to about 1.5 g, about 1.5 g to about 2 g, about 2 g to about 2.5 g, about 2.5 g to about 3 g, or about 3 g to about 3.5 g.

In embodiments, a subject receives sulfasalazine at maximum a dose of about 50 mg to about 1000 mg per day when co-administered with Compound 1. In embodiments, a subject receives sulfasalazine at maximum a dose of about 1 g to about 4 g per day when co-administered with Compound 1. In embodiments, a subject receives sulfasalazine at a maximum dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg per day when co-administered with Compound 1. In embodiments, a subject receives sulfasalazine at a maximum dose of about 1 g, about 1.1 g, about 1.2 g, about 1.3 g, about 1.4 g, about 1.5 g, about 1.6 g, about 1.7 g, about 1.8 g, about 1.9 g, about 2.0 g, about 2.1 g, about 2.2 g, about 2.3 g, about 2.4 g, about 2.5 g, about 2.6 g, about 2.7 g, about 2.8 g, about 2.9 g, about 3.0 g, about 3.1 g, 3.2 g, 3.2 g, 3.3 g, 3.4 g, 3.5 g, 3.6 g, 3.7 g, 3.8 g, 3.8 g, or 4.0 g per day when co-administered with Compound 1. In embodiments, a subject receives sulfasalazine at a maximum dose of about 500 mg per day when co-administered with Compound 1. In embodiments, a subject receives sulfasalazine at a maximum dose of about 350 mg to 500 mg per day when co-administered with Compound 1.

In certain embodiments, a sulfasalazine drug is administered continuously and/or indefinitely. In certain embodiments, a sulfasalazine drug is administered once, twice, or three times per day. In embodiments, a sulfasalazine drug is administered simultaneously with Compound 1. In embodiments, a sulfasalazine drug is not administered simultaneously with Compound 1.

In embodiments, a subject has renal anemia (anemia secondary to or associated with chronic kidney disease). In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid. In embodiments, the daily dose of {[5-(3-chlorophenyl)-3-hydroxypyridine-2-carbonyl]amino}acetic acid is increased. In embodiments, the subject is administered an initial daily dose of about 300 mg, 450 mg, or 600 mg of {[5-(3-chlorophenyl)-3-hydroxypyridine-2-carbonyl]amino}acetic acid.

Furosemide

In embodiments, a drug (e.g., a second drug) is a diuretic. In embodiments, a diuretic is furosemide. In embodiments, subject receives furosemide for treating or preventing hypervolemia, edema (including edema induced by chronic kidney disease, cardiovascular disease, or liver disease), and/or other swelling related to, e.g., congestive heart failure, liver disease, kidney disease, and other medical conditions. In embodiments, a subject receives furosemide at a dose of about 10 mg to about 80 mg per day. In embodiments, a subject receives furosemide at a dose of about 10 mg, about 20 mg, about 30 mg, about 40 mg, about 50 mg, about 60 mg, about 70 mg, or about 80 mg per day. In embodiments, a subject receives furosemide at a dose of about 40 mg per day. In embodiments, a daily dose is a maximum daily dose. In embodiments, a subject receives furosemide at a maximum dose of about 40 mg per day.

In embodiments, the dosage amount of a drug (e.g., a second drug) that is furosemide is not adjusted when co-administered with Compound 1. In embodiments, the dosage amount of a drug (e.g., a second drug) that is furosemide is adjusted when co-administered with Compound 1. In embodiments, the dosage amount is increased. In embodiments, the dosage amount is decreased. In embodiments, the daily dose is decreased by at least about 5 mg, about 10 mg, about 15 mg, about 20 mg, about 25 mg, about 30 mg, or about 35 mg. about 40 mg, about 45 mg, about 50 mg, about 55 mg, about 60 mg, about 65 mg, about 70 mg, about 75 mg. In embodiments, the daily dose is decreased by about 5 mg to about 15 mg, about 15 mg to about 25 mg, about 25 mg to about 35 mg, about 35 mg to about 45 mg, about 45 mg to about 55 mg, about 55 mg to about 65 mg, or about 65 mg to about 75 mg.

In embodiments, a subject receives furosemide at a maximum dose of about 10 mg to about 80 mg per day when co-administered with Compound 1. In embodiments, a subject receives furosemide at a maximum dose of about 10 mg, about 20 mg, about 30 mg, about 40 mg, about 50 mg, about 60 mg, about 70 mg, or about 80 mg per day when co-administered with Compound 1. In embodiments, a subject receives furosemide at a maximum dose of about 40 mg per day. In embodiments, a subject receives furosemide at a maximum dose of about 30 mg per day.

In certain embodiments, a furosemide drug is administered continuously and/or indefinitely. In certain embodiments, a furosemide drug is administered once, twice, or three times per day. In embodiments, a furosemide drug is administered simultaneously with Compound 1. In embodiments, a furosemide drug is not administered simultaneously with Compound 1.

In embodiments, a subject has renal anemia (anemia secondary to or associated with chronic kidney disease). In embodiments, the chronic kidney disease is dialysis-dependent chronic kidney disease (DD-CKD). In embodiments, the chronic kidney disease is non dialysis-dependent chronic kidney disease (NDD-CKD).

In embodiments, the subject is administered a daily dose of about 150-600 mg of {[5-(3-chlorophenyl)-3-hydroxypyridine-2-carbonyl]amino}acetic acid. In embodiments, the daily dose of {[5-(3-chlorophenyl)-3-hydroxypyridine-2-carbonyl]amino}acetic acid is increased. In embodiments, the subject is administered an initial daily dose of about 300, 450, or 600 mg of {[5-(3-chlorophenyl)-3-hydroxypyridine-2-carbonyl]amino}acetic acid.

Diseases Associated with HIF Prolyl Hydroxylase Modulation

In certain embodiments, the methods of the inventions include subjects having a disease associated with HIF prolyl hydroxylase modulation.

Diseases associated with HIF prolyl hydroxylase modulation include Peripheral Vascular Disease (PVD); Coronary Artery Disease (CAD); heart failure; ischemia; anemia; wound healing; ulcers; ischemic ulcers; inadequate blood supply; poor capillary circulation; small artery atherosclerosis; venous stasis; atherosclerotic lesions (e.g., in coronary arteries); angina; myocardial infarction; diabetes; hypertension; Buerger's disease; diseases associated with abnormal levels of VEGF, GAPDH, and/or EPO; Crohn's disease; ulcerative colitis; psoriasis; sarcoidosis; rheumatoid arthritis; hemangiomas; Osler-Weber-vasculitis disease; hereditary hemorrhagic telangiectasia; solid or blood borne tumors and acquired immune deficiency syndrome; atrial arrhythmias; ischemic tissue damage in tissues such as: cardiac tissue, such as myocardium and cardiac ventricles, skeletal muscle, neurological tissue, such as from the cerebellum, internal organs, such as the stomach, intestine, pancreas, liver, spleen, and lung; and distal appendages such as fingers and toes.

Specifically, the methods provided herein include administering Compound 1 and another drug (e.g., a second drug such as drugs comprising a multivalent cation, statin drugs, sulfasalazine, or furosemide) to a subject having, inter alia, cardiovascular toxicity, dyslipidemia, Peripheral Vascular Disease (PVD); Coronary Artery Disease (CAD); heart failure; ischemia; anemia; wound healing; ulcers; ischemic ulcers; inadequate blood supply; poor capillary circulation; small artery atherosclerosis; venous stasis; atherosclerotic lesions (e.g., in coronary arteries); angina; myocardial infarction; diabetes; hypertension; Buerger's disease; diseases associated with abnormal levels of VEGF, GAPDH, and/or EPO; Crohn's disease; ulcerative colitis; psoriasis; sarcoidosis; rheumatoid arthritis; hemangiomas; Osler-Weber-vasculitis disease; hereditary hemorrhagic telangiectasia; solid or blood borne tumors and acquired immune deficiency syndrome; atrial arrhythmias; ischemic tissue damage in tissues such as: cardiac tissue, such as myocardium and cardiac ventricles, skeletal muscle, neurological tissue, such as from the cerebellum, internal organs, such as the stomach, intestine, pancreas, liver, spleen, and lung; and distal appendages such as fingers and toes.

In certain embodiments, the methods provided herein include administering Compound 1 and another drug (e.g., a second drug such as drugs comprising a multivalent cation, statin drugs, sulfasalazine, or furosemide) to a subject having anemia, as such anemia secondary to non-dialysis dependent chronic kidney disease.

In embodiments, a subject has renal anemia (anemia secondary to or associated with chronic kidney disease).

In certain embodiments, the chronic kidney disease is stage 3, 4, or 5 chronic kidney disease. In certain embodiments, the chronic kidney disease is pre-dialysis chronic kidney disease. In other embodiments, the chronic kidney disease is non-dialysis dependent chronic kidney disease. In still other embodiments, the subject has not been previously treated for anemia, such as renal anemia (anemia secondary to or associated with chronic kidney disease). In alternative embodiments, the subject has been previously treated for anemia, such as renal anemia (anemia secondary to or associated with chronic kidney disease).

Doses and Dosing Regimens of Compound 1

The specific doses for uses of a Compound 1 can be administered in any manner known to the skilled artisan. Exemplary doses are provided herein, including in the Examples.

Doses of Compound 1 may be taken orally, topically, or intravenously. Doses of Compound 1 may be taken while fasting, together with fluids, or together with food of any kind. In specific embodiments, doses of Compound 1 may be taken or 1, 2, 3, 4, 5, 6, 7, 8, 9, 10, 11, or 12 hours after a meal, or 1, 2, 3, 4, 5, 6, 7, 8, 9, 10, 11, or 12 hours before a meal. Doses of Compound 1 may be taken at any time of day. In certain embodiments, repeat doses are administered at the same time during the day. In certain embodiments, the dose doses are administered in the morning, around mid-day, or in the evening. In certain embodiments, the doses are administered between 4:00 am and 2:00 pm. In certain embodiments, the doses are administered between 5:00 am and 1:00 pm. In certain embodiments, the doses are administered between 6:00 am and 12:00 noon. In certain embodiments, the doses are administered between 7:00 am and 11:00 am. In certain embodiments, the doses are administered between 8:00 am and 10:00 am. In certain embodiments, the doses are administered before, during, or after breakfast. Administration and dosing regimens may be adjusted as described herein.

Dose levels of the compound include 150, 300, 450, and 600 mg. Thereafter, the medication is taken once daily during the course of treatment. The subject should take the study medication with 4 ounces of water or other oral beverage, regardless of food intake. The dose is taken at approximately the same time each day, preferably between 7 AM and 2 PM.

In a specific embodiment, a subject is initially treated with 300 mg of Compound 1 daily (300 mg/day).

In a specific embodiment, a subject is initially treated with 450 mg of Compound 1 daily (450 mg/day).

In a specific embodiment, a subject is initially treated with 600 mg of Compound 1 daily (600 mg/day).

This section provides several exemplary doses for Compound 1. In certain embodiments, such a dose is the initial dose at the beginning of a treatment. In other embodiments, such a dose is the adjusted dose at a later time during the course of treatment.

In certain embodiments, the daily dose of Compound 1 is between about 150 mg and about 600 mg. In certain embodiments, the daily dose of the compound is between about 150 mg and about 300 mg or about 300 and about 600 mg. In certain embodiments, the daily dose is about 150 mg, 200 mg, 250 mg, 300 mg, 350 mg, 400 mg, 450 mg, 500 mg, 550 mg, or 600 mg of Compound 1, or a pharmaceutically acceptable salt thereof. In certain embodiments, the daily dose of Compound 1, or a pharmaceutically acceptable salt thereof, is at least about 150 mg, at least about 300 mg, at least about 450 mg, or even at least about 600 mg.

In certain embodiments, the daily dose is about 150 mg, about 300 mg, about 450 mg, or about 600 mg of Compound 1. In certain embodiments, the daily dose Compound 1 is about 150 mg, about 300 mg, about 450 mg, or about 600 mg. In embodiments, a maximum dose is about 600 mg.

In embodiments, a starting dose is about 300 mg, and the dose is adjusted (e.g., according to the patient's condition). In embodiments, a maximum dose is about 600 mg.

In embodiments, a starting dose is about 450 mg, and the dose is adjusted (e.g., according to the patient's condition). In embodiments, a maximum dose is about 600 mg.

In certain embodiments, a daily dose of 450 mg of Compound 1 may be decreased by about 150 mg, such that the daily dose of the compound is about 300 mg. In certain embodiments, a daily dose of Compound 1 may be decreased by about 300 mg, such that the daily dose of the compound is about 150 mg. In certain embodiments, the daily dose Compound 1 may be increased or decreased by about 75 mg, about 100 mg, about 125 mg, about 150 mg, about 175 mg, about 200 mg, about 225 mg, about 250 mg, about 275 mg, or about 300 mg. In certain embodiments, the daily dose may be increased or decreased by an amount between about 75 mg and 300 mg, about 100 mg and about 300 mg, about 125 mg and about 300 mg, about 150 mg and about 300 mg, about 175 mg and about 300 mg, about 200 mg and about 300 mg, about 225 mg and about 300 mg, about 250 mg and about 300 mg, or about 275 mg and about 300 mg. In certain embodiments, the daily dose of Compound 1, or a pharmaceutically acceptable salt thereof, may be increased or decreased by an amount between about 75 mg and about 250 mg, about 100 mg and about 225 mg, or about 125 mg and about 200 mg. In certain such embodiments, the daily dose Compound 1 does not exceed about 600 mg.

Dose Adjustments of Compound 1

In certain embodiments, the dose may be adjusted by administering to a patient having anemia an initial daily dose of a compound which is {[5-(3-chlorophenyl)-3-hydroxypyridine-2-carbonyl]amino}acetic acid (Compound 1) or a pharmaceutically acceptable salt, solvate, or hydrate thereof administered in a formulation as described herein; taking a first measurement of the hemoglobin level in the patient and subsequently taking a second measurement of the hemoglobin level in the patient, wherein if the hemoglobin level in the patient at the second measurement is less than about 10.0 g/dL and the level of hemoglobin has decreased by less than about 0.5 g/dL as compared to the level at the first measurement; or if the hemoglobin level in the patient at the second measurement is less than about 10.0 g/dL and the level of hemoglobin has changed by up to about 0.4 g/dL as compared to the level at the first measurement; or if the hemoglobin level in the patient at the second measurement is between about 10.0 and about 10.9 g/dL and the level of hemoglobin has decreased by less than about 0.5 g/dL as compared to the level at the first measurement; then administering an adjusted daily dose of the compound that is greater than the initial daily dose. In certain such embodiments, the adjusted daily dose of the compound is about 150 mg greater than the initial daily dose.

In certain embodiments, the dose may be adjusted by administering to a patient having anemia an initial daily dose of a compound which is {[5-(3-chlorophenyl)-3-hydroxypyridine-2-carbonyl] amino} acetic acid, or a pharmaceutically acceptable salt thereof, administered in a formulation as described herein; taking a first measurement of the hemoglobin level in the patient and subsequently taking a second measurement of the hemoglobin level in the patient, wherein if the hemoglobin level in the patient at the second measurement is less than about 10.0 g/dL and the level of hemoglobin has increased by greater than about 1.5 g/dL as compared to the level at the first measurement; or if the hemoglobin level in the patient at the second measurement is between about 10.0 and about 10.9 g/dL and the level of hemoglobin has increased by greater than about 1.5 g/dL as compared to the level at the first measurement; or if the hemoglobin level in the patient at the second measurement is between about 11.0 and about 12.2 g/dL and the level of hemoglobin has increased by between about 1.0 and about 1.4 g/dL as compared to the level at the first measurement; or if the hemoglobin level in the patient at the second measurement is between about 12.3 and about 12.9 g/dL and the level of hemoglobin has decreased by up to about 0.4 g/dL or increased by up to about 0.4 g/dL as compared to the at the first measurement; or if the hemoglobin level in the patient at the second measurement is between about 12.3 and about 12.9 g/dL and the level of hemoglobin has increased by about 0.5 to about 0.9 g/dL as compared to the level at the first measurement; then administering an adjusted daily dose of the compound that is less than the initial daily dose. In certain such embodiments, the adjusted daily dose of the compound is about 150 mg less than the initial daily dose. In certain embodiments, the lowest dose level is 150 mg per day. Patients already on the lowest dose level will continue on 150 mg per day unless their Hgb increases to ≥13.0 g/dL.

In certain embodiments, the dose may be adjusted by administering to a patient having anemia an initial daily dose of a compound which is {[5-(3-chlorophenyl)-3-hydroxypyridine-2-carbonyl]amino}acetic acid (Compound 1) or a pharmaceutically acceptable salt thereof, administered in a formulation; taking a first measurement of the hemoglobin level in the patient and subsequently taking a second measurement of the hemoglobin level in the patient, wherein if the hemoglobin level in the patient at the second measurement is between about 11.0 and about 12.2 g/dL and the level of hemoglobin has increased by greater than about 1.5 g/dL as compared to the level at the first measurement; or if the hemoglobin level in the patient at the second measurement is between about 12.3 and about 12.9 g/dL and the level of hemoglobin has increased by between about 1.0 and about 1.4 g/dL as compared to the level at the first measurement; or if the hemoglobin level in the patient at the second measurement is between about 12.3 and about 12.9 g/dL and the level of hemoglobin has increased by greater than about 1.5 g/dL as compared to the level at the first measurement; then administering an adjusted daily dose of the compound that is less than the initial daily dose. In certain such embodiments, the adjusted daily dose of the compound is about 300 mg less than the initial daily dose. In certain embodiments, the lowest dose level is 150 mg per day. Patients already on the lowest dose level will continue on 150 mg per day unless their Hgb increases to 13.0 g/dL.

In certain embodiments, the dose may be adjusted by administering to a patient having anemia an initial daily dose of a compound which is {[5-(3-chlorophenyl)-3-hydroxypyridine-2-carbonyl]amino}acetic acid (Compound 1) or a pharmaceutically acceptable salt thereof, administered in a formulation; taking a first measurement of the hemoglobin level in the patient and subsequently taking a second measurement of the hemoglobin level in the patient, wherein if the hemoglobin level in the patient at the second measurement is equal to or above 13.0 g/dL, then administering an adjusted daily dose of the compound that is less than the initial daily dose. In certain such embodiments, dosing is suspended. In certain such embodiments, dosing will be suspended if Hgb rises to 13 g/dL, and will not be restarted until Hgb reduces to 12.5 g/dL. Factors that may temporarily change the Hgb level should be considered before suspending the dose. Hgb is assessed every 2 weeks during this time period.

In certain embodiments, the dose may be adjusted by administering to a patient having anemia an initial daily dose of a compound which is {[5-(3-chlorophenyl)-3-hydroxypyridine-2-carbonyl]amino}acetic acid (Compound 1) or a pharmaceutically acceptable salt thereof; taking a first measurement of the hemoglobin level in the patient and subsequently taking a second measurement of the hemoglobin level in the patient, wherein if the hemoglobin level in the patient at the second measurement is equal to or above 12.5 g/dL, then administering an adjusted daily dose of the compound that is less than the initial daily dose. In certain such embodiments, dosing is suspended. In certain such embodiments, dosing will be suspended if Hgb rises to 12.5 g/dL, and will not be restarted until Hgb reduces to 12.0 g/dL. Factors that may temporarily change the Hgb level should be considered before suspending the dose. Hgb is assessed every 2 weeks during this time period.

In certain embodiments, the dose may be adjusted by administering to a patient having anemia an initial daily dose of a compound which is {[5-(3-chlorophenyl)-3-hydroxypyridine-2-carbonyl]amino}acetic acid (Compound 1) or a pharmaceutically acceptable salt thereof; taking a first measurement of the hemoglobin level in the patient and subsequently taking a second measurement of the hemoglobin level in the patient, wherein if the hemoglobin level in the patient at the second measurement is equal to or above 13.0 g/dL if the patient is an adult male or 12.5 g/dL if the patient is an adult female, then administering an adjusted daily dose of the compound that is less than the initial daily dose. In certain such embodiments, dosing is suspended. In certain such embodiments, dosing will be suspended if Hgb rises to 13.0 g/dL if the patient is an adult male or to 12.5 g/dL if the patient is an adult female, and will not be restarted until Hgb reduces to 12.5 g/dL if the patient is an adult male or 12.0 g/dL if the patient is an adult female. Factors that may temporarily change the Hgb level should be considered before suspending the dose. Hgb is assessed every 2 weeks during this time period.

In certain embodiments, the dose may be adjusted by administering to a patient having anemia an initial daily dose of a compound which is {[5-(3-chlorophenyl)-3-hydroxypyridine-2-carbonyl]amino}acetic acid (Compound 1) or a pharmaceutically acceptable salt thereof, administered in a formulation; taking a first measurement of the hemoglobin level in the patient and subsequently taking a second measurement of the hemoglobin level in the patient, wherein if the hemoglobin level in the patient at the second measurement is less than about 9.5 to 10.5 g/dL or about 9.75 to 10.25 g/dL and the level of hemoglobin has decreased by less than about 0.2 to 0.8, about 0.3 to 0.7, or about 0.4 to 0.6 g/dL as compared to the level at the first measurement; or if the hemoglobin level in the patient at the second measurement is less than about 9.5 to 10.5 g/dL or about 9.75 to 10.25 g/dL and the level of hemoglobin has changed by up to about 0.1 to 0.7, about 0.2 to 0.6, or about 0.3 to 0.5 g/dL as compared to the level at the first measurement; or if the hemoglobin level in the patient at the second measurement is between about 9.5 to 10.5 g/dL or about 9.75 to 10.25 g/dL and about 10.4 to 11.4 g/dL or about 10.65 to 11.15 g/dL and the level of hemoglobin has decreased by less than about 0.2 to 0.8, about 0.3 to 0.7, or about 0.4 to 0.6 g/dL as compared to the level at the first measurement; then administering an adjusted daily dose of the compound that is greater than the initial daily dose. In certain such embodiments, the adjusted daily dose of the compound is about 150 mg greater than the initial daily dose.

In certain embodiments, the dose may be adjusted by administering to a patient having anemia an initial daily dose of a compound which is {[5-(3-chlorophenyl)-3-hydroxypyridine-2-carbonyl] amino} acetic acid or a pharmaceutically acceptable salt thereof, administered in a formulation; taking a first measurement of the hemoglobin level in the patient and subsequently taking a second measurement of the hemoglobin level in the patient, wherein if the hemoglobin level in the patient at the second measurement is less than about 9.5 to about 10.5 g/dL or about 9.75 to about 10.25 g/dL and the level of hemoglobin has increased by greater than about 1.2 to about 1.8, about 1.3 to about 1.7, or about 1.4 to about 1.6 g/dL as compared to the level at the first measurement; or if the hemoglobin level in the patient at the second measurement is between about 9.5 to about 10.5 or about 9.75 to about 10.25 g/dL and about 10.4 to about 11.4 g/dL or about 10.65 to about 11.15 g/dL and the level of hemoglobin has increased by greater than about 1.2 to about 1.8, about 1.3 to about 1.7, or about 1.4 to about 1.6 g/dL as compared to the level at the first measurement; or if the hemoglobin level in the patient at the second measurement is between about 10.5 to about 11.5 g/dL or about 10.75 to about 11.25 g/dL and about 11.7 to about 12.7 g/dL or about 11.95 to about 12.45 g/dL g/dL and the level of hemoglobin has increased by between about 0.7 to about 1.3, about 0.8 to about 1.2, or about 0.9 to about 1.1 g/dL, and about 1.1 to about 1.7, about 1.2 to about 1.6, or about 1.3 to about 1.5 g/dL as compared to the level at the first measurement; or if the hemoglobin level in the patient at the second measurement is between about 11.8 to about 12.8 g/dL or about 12.05 to about 12.55 g/dL and about 12.4 to about 13.9 g/dL or about 12.65 to about 13.15 g/dL and the level of hemoglobin has decreased by up to about 0.1 to about 0.7, about 0.2 to about 0.6, or about 0.3 to about 0.5 g/dL or increased by up to about 0.1 to about 0.7, about 0.2 to about 0.6, or about 0.3 to about 0.5 g/dL as compared to the at the first measurement; or if the hemoglobin level in the patient at the second measurement is between about 11.8 to about 12.8 g/dL, or about 12.05 to about 12.55 g/dL and about 12.4 to about 13.9 g/dL or about 12.65 to about 13.15 g/dL g/dL and the level of hemoglobin has increased by about 0.2 to about 0.8 g/dL, about 0.3 to about 0.7 g/dL, about 0.4 to about 0.6 g/dL, about 0.6 to about 1.2 g/dL, about 0.7 to about 1.1 g/dL, or about 0.8 to 1.0 g/dL as compared to the level at the first measurement; then administering an adjusted daily dose of the compound that is less than the initial daily dose. In certain such embodiments, the adjusted daily dose of the compound is about 150 mg less than the initial daily dose. In certain embodiments, the lowest dose level is 150 mg per day. Patients already on the lowest dose level will continue on 150 mg per day unless their Hgb increases to >12.0 g/dL, 12.5 g/dL, or 13.0 g/dL.

In certain embodiments, the dose may be adjusted by administering to a patient having anemia an initial daily dose of a compound which is {[5-(3-chlorophenyl)-3-hydroxypyridine-2-carbonyl]amino}acetic acid (Compound 1) or a pharmaceutically acceptable salt thereof, administered in a formulation; taking a first measurement of the hemoglobin level in the patient and subsequently taking a second measurement of the hemoglobin level in the patient, wherein if the hemoglobin level in the patient at the second measurement is between about 10.5 to 11.5 g/dL, or about 10.75 to 11.25 g/dL and about 11.7 to 12.7 g/dL or about 11.95 to 12.45 g/dL g/dL and the level of hemoglobin has increased by greater than about 1.2 to 1.8 g/dL, about 1.3 to about 1.7 g/dL, or about 1.4 to about 1.6 g/dL as compared to the level at the first measurement; or if the hemoglobin level in the patient at the second measurement is between about 11.8 to about 12.8 g/dL or about 12.05 to about 12.55 g/dL and about 12.4 to about 13.9 g/dL or about 12.65 to about 13.15 g/dL g/dL and the level of hemoglobin has increased by between about 0.7 to about 1.3 g/dL, about 0.8 to about 1.2 g/dL, or about 0.9 to about 1.1 g/dL and about 1.1 to about 1.7 g/dL, about 1.2 to about 1.6 g/dL, or about 1.3 to about 1.5 g/dL as compared to the level at the first measurement; or if the hemoglobin level in the patient at the second measurement is between about 11.8 to about 12.8 g/dL or about 12.05 to about 12.55 g/dL and about 12.4 to about 13.9 g/dL or about 12.65 to about 13.15 g/dL and the level of hemoglobin has increased by greater than about 1.2 to about 1.8 g/dL, about 1.3 to about 1.7 g/dL, or about 1.4 to about 1.6 g/dL as compared to the level at the first measurement; then administering an adjusted daily dose of the compound that is less than the initial daily dose. In certain such embodiments, the adjusted daily dose of the compound is about 300 mg less than the initial daily dose. In certain embodiments, the lowest dose level is 150 mg per day. Patients already on the lowest dose level will continue on 150 mg per day unless their Hgb increases to ≥12.0 g/dL, 12.5 g/dL, or 13.0 g/dL.

In certain embodiments, the dose may be adjusted by administering to a patient having anemia an initial daily dose of a compound which is {[5-(3-chlorophenyl)-3-hydroxypyridine-2-carbonyl]amino}acetic acid (Compound 1) or a pharmaceutically acceptable salt thereof, administered in a formulation; taking a first measurement of the hemoglobin level in the patient and subsequently taking a second measurement of the hemoglobin level in the patient, wherein if the hemoglobin level in the patient at the second measurement is equal to or above 11.0 g/dL, 11.5 g/dL, 12.0 g/dL, 12.5 g/dL, 13.0 g/dL, 13.5 g/dL, or 14.0 g/dL, then administering an adjusted daily dose of the compound that is less than the initial daily dose. In certain such embodiments, dosing is suspended. In certain such embodiments, dosing will be suspended if Hgb rises to 11.0 g/dL, 11.5 g/dL, 12.0 g/dL, 12.5 g/dL, 13.0 g/dL, 13.5 g/dL, or 14.0 g/dL, and will not be restarted until Hgb reduces to 10.5 g/dL, 11.0 g/dL, 11.5 g/dL, 12.0 g/dL, 12.5 g/dL, 13.0 g/dL, or 13.5 g/dL. Factors that may temporarily change the Hgb level should be considered before suspending the dose. Hgb is assessed every 2 weeks during this time period.

In certain embodiments, the dose may be adjusted by administering to a patient having anemia an initial daily dose of a compound which is {[5-(3-chlorophenyl)-3-hydroxypyridine-2-carbonyl]amino}acetic acid (Compound 1) or a pharmaceutically acceptable salt thereof, administered in a formulation; taking a first measurement of the hemoglobin level in the patient and subsequently taking a second measurement of the hemoglobin level in the patient, wherein if the hemoglobin level in the patient at the second measurement is equal to or above 10.5 g/dL, 11.0 g/dL, 11.5 g/dL, 12.0 g/dL, 12.5 g/dL, 13.0 g/dL, or 13.5 g/dL, then administering an adjusted daily dose of the compound that is less than the initial daily dose. In certain such embodiments, dosing is suspended. In certain such embodiments, dosing will be suspended if Hgb rises to 10.5 g/dL, 11.0 g/dL, 11.5 g/dL, 12.0 g/dL, 12.5 g/dL, 13.0 g/dL, or 13.5 g/dL, and will not be restarted until Hgb reduces to 10.0 g/dL, 10.5 g/dL, 11.0 g/dL, 11.5 g/dL, 12.0 g/dL, 12.5 g/dL, or 13.0 g/dL. Factors that may temporarily change the Hgb level should be considered before suspending the dose. Hgb is assessed every 2 weeks during this time period.

In certain embodiments, the dose may be adjusted by administering to a patient having anemia an initial daily dose of a compound which is {[5-(3-chlorophenyl)-3-hydroxypyridine-2-carbonyl]amino}acetic acid (Compound 1) or a pharmaceutically acceptable salt thereof, administered in a formulation; taking a first measurement of the hemoglobin level in the patient and subsequently taking a second measurement of the hemoglobin level in the patient, wherein if the hemoglobin level in the patient at the second measurement is equal to or above 11.0, 11.5, 12.0, 12.5, 13.0, 13.5, or 14.0 g/dL if the patient is an adult male or 10.5 g/dL, 11.0 g/dL, 11.5 g/dL, 12.0 g/dL, 12.5 g/dL, 13.0 g/dL, or 13.5 g/dL if the patient is an adult female, then administering an adjusted daily dose of the compound that is less than the initial daily dose. In certain such embodiments, dosing is suspended. In certain such embodiments, dosing will be suspended if Hgb rises to 11.0 g/dL, 11.5 g/dL, 12.0 g/dL, 12.5 g/dL, 13.0 g/dL, 13.5 g/dL, or 14.0 g/dL if the patient is an adult male or to 10.5 g/dL, 11.0 g/dL, 11.5 g/dL, 12.0 g/dL, 12.5 g/dL, 13.0 g/dL, or 13.5 g/dL if the patient is an adult female, and will not be restarted until Hgb reduces to 10.5 g/dL, 11.0 g/dL, 11.5 g/dL, 12.0 g/dL, 12.5 g/dL, 13.0 g/dL, or 13.5 g/dL if the patient is an adult male or 10.0 g/dL, 10.5 g/dL, 11.0 g/dL, 11.5 g/dL, 12.0 g/dL, 12.5 g/dL, or 13.0 g/dL if the patient is an adult female. Factors that may temporarily change the Hgb level should be considered before suspending the dose. Hgb is assessed every 2 weeks during this time period. The dose adjustment methods described herein may be applied in treatment regimens using any compound described herein, or any combination thereof.

Dose Adjustment based on Hemoglobin Levels

In certain embodiments, the dose may be adjusted by administering to a patient having anemia an initial daily dose of Compound 1 administered in a formulation, taking a first measurement of the hemoglobin level in the patient and subsequently taking a second measurement of the hemoglobin level in the patient, and adjusting the dose as described below. In certain embodiments, the first measurement is a baseline measurement. In certain embodiments, the dose may be adjusted by administering to a patient having anemia an initial daily dose of a compound which is Compound 1 administered in a formulation as described herein; taking a first measurement of the hemoglobin level in the patient and subsequently taking a second measurement of the hemoglobin level in the patient; and adjusting the dose as described below.

In certain embodiments, hemoglobin levels can be determined and monitored, e.g., via a HemoCue® point of care Hgb monitoring system, throughout the study to determine if the dose of study medication will be adjusted. In certain embodiments, Hgb can be obtained via HemoCue® every 2 weeks for monitoring for dose adjustment. In certain embodiments, Hgb can be obtained via HemoCue® every 4 weeks, unless more frequent monitoring is clinically indicated or warranted based on dosing changes. In certain embodiments, Hgb can be obtained via HemoCue® every 4, 6, 8, 10, 12, or 16 weeks. In certain embodiments, hemoglobin can also be assessed with a complete blood count (CBC) through the central laboratory for efficacy and safety evaluations; however, dose adjustments are based on the local HemoCue® Hgb value. In certain embodiments, the aim is to increase and maintain a Hgb level of 10-11 g/dL. In certain embodiments, the aim is to increase and maintain a Hgb level of 10-12 g/dL. In certain embodiments, the aim is to increase and maintain a Hgb level of 10-13 g/dL.

In certain embodiments, Compound 1 is dosed according to the following dose-adjustment algorithm guidelines. When adjusting therapy, Hgb rate of rise, rate of decline, and variability is considered. A single Hgb excursion may not require a dosing change.

Dose Adjustment Procedure in Patients with Chronic Kidney Disease

In certain embodiments, the dosing of a Compound 1 is adjusted during the course of treatment of a patient as described below. In certain specific embodiments, the dose is adjusted to correct anemia in a patient. In certain specific embodiments, the patient has non-dialysis dependent chronic kidney disease (NDD-CKD).

In certain embodiments, a baseline value is determined immediately prior to the first administration of Compound 1. In certain embodiments, the initial daily dose administered to the patients is 300 mg/day. In certain specific embodiments, the initial daily dose is administered in form of two tablets of 150 mg each. In certain embodiments, the initial daily dose administered to the patients is 450 mg/day. In certain specific embodiments, the initial daily dose is administered in form of three tablets of 150 mg each. In certain specific embodiments, the initial daily dose is administered in the morning. In certain specific embodiments, the initial daily dose is administered between 7 am and 2 pm.

In certain embodiments, the daily dose of Compound 1 is not increased more frequently than once every 4 weeks during the course of treatment. Decreases in daily dose can occur more frequently, but frequent dose adjustments are to be avoided.

In certain embodiments, if the Hgb has not increased by more than 0.5 g/dL above the baseline value after 4 weeks of treatment, the daily dose of the compound is increased by 150 mg/day. The daily dose is increased by 150 mg/day every 4 weeks until Hgb is above 10.0 g/dL (maximum dose is 600 mg/day). In certain specific embodiments, if the Hgb has not increased by more than 0.5 g/dL above the baseline value after 4 weeks of treatment of an NDD-CKD patient with a daily dose of Compound 1, the daily dose of the compound is increased by 150 mg/day. In certain specific embodiments, the daily dose of Compound 1 is increased by 150 mg/day every 4 weeks until Hgb in the NDD-CKD patient is above 10.0 g/dL (maximum dose is 600 mg/day).

In certain embodiments, if the Hgb rises rapidly during treatment (e.g., more than 1.0 g/dL in any 2-week period), the daily dose is reduced by 150 mg/day. In certain specific embodiments, if the Hgb in an NDD-CKD patient rises rapidly during treatment with a daily dose of Compound 1(e.g., more than 1.0 g/dL in any 2-week period), the daily dose is reduced by 150 mg/day.

In certain embodiments, if the Hgb falls below 10.0 g/dL, the daily dose is increased by 150 mg/day. In certain specific embodiments, if the Hgb in an NDD-CKD patient falls below 10.0 g/dL during treatment with a dose of Compound 1, the daily dose is increased by 150 mg/day.

In certain embodiments, if the Hgb level exceeds 11.0 g/dL, treatment is interrupted until the Hgb decreases to 10.5 g/dL or less. Thereafter, dosing is resumed with a daily dose reduced by 150 mg/day. In certain specific embodiments, if the Hgb level in an NDD-CKD patient exceeds 11.0 g/dL, treatment with Compound 1 is interrupted until the Hgb decreases to 10.5 g/dL or less. Thereafter, dosing with Compound 1 is resumed with a daily dose reduced by 150 mg/day.

In certain embodiments, if the Hgb level exceeds 12.0 g/dL, the daily dose is reduced by 150 mg. In certain embodiments, if the Hgb level exceeds 13.0 g/dL, treatment is interrupted until the Hgb decreases to 12.5 g/dL or less. Thereafter, dosing is resumed with with a daily dose reduced by 150 mg/day. In certain specific embodiments, if the Hgb level in an NDD-CKD patient exceeds 12.0 g/dL during treatment with a daily dose of Compound 1, the dose is reduced by 150 mg/day. In certain specific embodiments, if the Hgb level in an NDD-CKD patient exceeds 13.0 g/dL, treatment with Compound 1 is interrupted until the Hgb decreases to 12.5 g/dL or less. Thereafter, dosing with Compound 1 is resumed with a daily dose reduced by 150 mg/day.

In certain embodiments, if a dose adjustment is required to maintain Hgb at the desired level, the daily dose is adjusted by 150 mg/day. In certain specific embodiments, if a dose adjustment of Compound 1 is required to maintain Hgb in an NDD-CKD patient at the desired level, the daily dose is adjusted by 150 mg/day.

In certain embodiments, the dosing of Compound 1, is adjusted during the course of treatment of a patient as described below. In certain specific embodiments, the daily dose is adjusted for the maintenance treatment of anemia in a patient. In certain specific embodiments, the patient has non-dialysis dependent chronic kidney disease (NDD-CKD).

In certain embodiments, a baseline value is determined immediately prior to the first administration of Compound 1. In certain embodiments, the initial daily dose administered to the patients is 300 mg/day. In certain specific embodiments, the initial daily dose is administered in form of two tablets of 150 mg each. In certain embodiments, the initial daily dose administered to the patients is 450 mg/day. In certain specific embodiments, the initial daily dose is administered in form of three tablets of 150 mg each. In certain specific embodiments, the initial daily dose is administered in the morning. In certain specific embodiments, the initial daily dose is administered between 7 am and 2 pm.

In certain embodiments, the daily dose of Compound 1 is not increased more frequently than once every 4 weeks during the course of treatment. Decreases in daily dose can occur more frequently, but frequent dose adjustments are to be avoided.

In certain embodiments, if dose adjustment is required to maintain Hgb at the desired level, the daily dose of the compound is adjusted by 150 mg/day (maximum daily dose is 600 mg/day). In certain specific embodiments, if dose adjustment is required to maintain Hgb at the desired level in an NDD-CKD patient, the daily dose of Compound 1 is adjusted by 150 mg/day (maximum dose is 600 mg/day).

In certain embodiments, if the Hgb falls below 10.0 g/dL, the daily dose is increased by 150 mg/day. In certain specific embodiments, if the Hgb in an NDD-CKD patient falls below 10.0 g/dL during treatment with a dose of Compound 1, the daily dose is increased by 150 mg/day.

In certain embodiments, if the Hgb level exceeds 11.0 g/dL, treatment is interrupted until the Hgb decreases to 10.5 g/dL or less. Thereafter, dosing is resumed with a daily dose reduced by 150 mg/day. In certain specific embodiments, if the Hgb level in an NDD-CKD patient exceeds 11.0 g/dL, treatment with Compound 1 is interrupted until the Hgb decreases to 10.5 g/dL or less. Thereafter, dosing with Compound 1 is resumed with a daily dose reduced by 150 mg/day.

In certain embodiments, if the Hgb level exceeds 12.0 g/dL, the daily dose is reduced by 150 mg/day. In certain embodiments, if the Hgb level exceeds 13.0 g/dL, treatment is interrupted until the Hgb decreases to 12.5 g/dL or less. Thereafter, dosing is resumed with with a daily dose reduced by 150 mg/day. In certain specific embodiments, if the Hgb level in an NDD-CKD patient exceeds 12.0 g/dL during treatment with a daily dose of Compound 1, the daily dose is reduced by 150 mg/day. In certain specific embodiments, if the Hgb level in an NDD-CKD patient exceeds 13.0 g/dL, treatment with Compound 1 is interrupted until the Hgb decreases to 12.5 g/dL or less. Thereafter, dosing with Compound 1 is resumed with a daily dose reduced by 150 mg/day.

### EXEMPLIFICATION

### Example 1: Clinical Information Related to Compound 1

Mechanism of Action

Under normal oxygen concentration, prolyl hydroxylase (PHD) causes hypoxia inducible factor (HIF) a to be hydroxylated and decomposed, while PHD activity is decreased under low oxygen concentration, HIF-α protein is stabilized. As a result, gene expression including erythropoietin is increased, and erythropoiesis is enhanced to obtain adaptability to a hypoxic environment. Without being bound by theory, by inhibiting PHD activity, Compound 1 mimics the above-mentioned biological response, and exerts hemoglobin production and erythropoiesis by increasing erythropoietin production following stabilization of HIF-α protein.

PHD Inhibitory Action

Compound 1 inhibits human PHD1, PHD2, and PHD3 (IC₅₀ values: 15.36 nmol/L, 11.83 nmol/L, and 7.63 nmol/L, respectively) (in vitro).

Blood Erythropoietin Concentration Increasing Action

In a normal rat, a single oral administration of Compound 1 showed elevated blood erythropoietin levels.

Hemoglobin Concentration

In normal rats, repeated oral administration of Compound 1 for 14 days showed the effect of increasing hemoglobin concentration and red blood cell count.

Guidelines for Initiation of Administration of Compound 1

As a guideline for initiation of administration, hemoglobin concertation in hemodialysis patients is less than 10 g/dL (30% in hematocrit value), hemoglobin concentration should be less than 11 g/dL (33% hematocrit) in patients with chronic kidney disease and peritoneal dialysis patients in the storage stage.

When administering this drug, it should be confirmed that that the patient has renal anemia, and it should not be administered for other anemias, such as blood loss anemia, pancytopenia, etc.

During administration of this drug, the hemoglobin concentration or hematocrit value should be observed regularly.

Dosage Regimens

Adult patients are typically administered with an initial dose of 300 mg of Compound 1 orally, once daily.

Once an anemia-ameliorating effect is obtained, adult patients typically are administered 150 mg-600 mg of Compound 1 orally, once daily.

The dose may be adjusted appropriately according to factors including the progress and severity of anemia. The dose may also be adjusted appropriate based on, for example, the dose of a red blood cell (RBC) stimulating agent (e.g., an erythropoiesis-stimulating agent) preparation before switching. However, the maximum once daily dose of Compound 1 is 600 mg.

Increase or decrease the dose in the range of 150-600 mg daily if dose adjustment may be necessary, where no proper increase is seen in hemoglobin or hematocrit level in the early administration and/or where it is difficult to maintain hemoglobin or hematocrit level in the target range in the maintenance administration period. Dose increases or reductions are considered before the hemoglobin or hematocrit level deviate from the target values by fully monitoring changes in the level. However, the dose should not be increased by more than 150 mg increments.

Administration of an iron agent may be performed if iron deficiency is observed.

During administration of Compound 1, the hemoglobin concentration or hematocrit value are observed regularly to prevent excessive hematopoietic activity. Complications, including heart failure, ischemic heart disease, and death are associate with hemoglobin concentrations round 14 g/dL (hematocrit level 42%) compared to those with hemoglobin concentrations round 10 g/dL (hematocrit level 30%).

Pharmacokinetics of Compound 1

Blood levels-Single Dose

Changes in plasma concentration and pharmacokinetic parameters on the first day of administration when 150 mg, 300 mg, and 600 mg of Compound 1 are repeatedly administered once a day for 10 days to healthy adult men are summarized in Table 3.

TABLE 3
Repeated Dose Administration

| | AUC₀₋ₗₐₛₜ | Cₘₐₓ | tₘₐₓ | t_{1/2} |
|---|---|---|---|---|
| Dose | (μg · h/mL) | (μg/mL) | (h) | (h) |
| 150 mg | 123 ± 30.5 | 24.2 ± 4.99 | 0.750 | 5.96 ± 0.914 |
| | | | (0.450-3.93) | |
| 300 mg | 289 ± 75.3 | 44.3 ± 10.8 | 1.95 | 6.14 ± 0.763 |
| | | | (1.99-4.00) | |
| 600 mg | 624 ± 205 | 84.8 ± 22.3 | 1.98 | 6.07 ± 0.419 |
| | | | (0.98-4.00) | |

Ratio (%) of the geometric mean of Cₘₐₓ and AUC_{0-inf}. when fasted or fed a single dose of 450 mg of this drug to healthy adult males) and its 90% confidence intervals were 73% [68%, 79%] and 94% [90%, 98%]. The median tₘₐₓ of this drug was prolonged by postprandial administration for about 1.5 hours as compared with fasting. These results are summarized in Table 4.

TABLE 4
Influences of Meal

| | Cₘₐₓ | AUC_{0-inf} | tₘₐₓ |
|---|---|---|---|
| Dose | (μg/mL) | (μg · h/mL) | (h) |
| Fasting | 63.1 ± 14.58 | 371 ± 100.0 | 2.00 |
| | | | (0.97-6.00) |
| After Meal | 46.3 ± 12.17 | 351 ± 101.3 | 3.52 |
| | | | (1.03-8.97) |

Distribution

The human plasma protein binding rate of this drug was higher than 99% (in vitro, equilibrium dialysis).

Metabolism

Compound 1 is metabolized via UGT, mainly producing O-glucuronide conjugates. When Compound 1 was orally administered to a healthy adult male (6 patients) once with 650 mg of [¹⁴C]-labeled, Compound 1 accounts for 75% of the total radioactivity (AUC_{0-∞}) in plasma, whereas the O-glucuronide conjugate was about 15%.

UGT1A1, UGT1A7, UGT1A8 and UGT1A9 were involved in the generation of O-glucuronide, which is the main metabolite of this drug in humans (in vitro). Although Compound 1 showed an inhibitory effect on CYP2B6, CYP2C8, CYP2C9, and UGT1A1 (in vitro), the variation ratio of AUC of each typical substrate was less than 1.25 times (static) Pharmacokinetics model).

Excretion

When a single dose of 650 mg of [¹⁴ C]-labeled Compound 1 was administered to healthy adult males (6 cases), 58.9% of the total radioactivity administered is in urine and 26 9% in feces by 72 hours after administration. The urinary excretion rate of this drug and acyl glucuronide is less than 1% of the total radioactivity. Without being bound by theory, the main excretion route of this Compound 1 is urinary excretion of O-glucuronide conjugate via glucuronidation.

Phase III Clinical Trial of Compound 1 with Patients Having Hemodialysis Anemia

Compound 1 was administered for 24 weeks to patients with hemodialysis anemia who did not use erythrocyte stimulating factor (the dose of Compound 1 was 300 mg once daily as the initial dose and 150 mg to 600 mg once daily as the maintenance dose). Average hemoglobin levels at 20 and 24 weeks are shown in Table 5. The incidence of adverse reactions when this drug was administered up to 24 weeks was 8.3% (2/24 cases).

TABLE 5
Phase III Clinical Trial of Compound 1

| | Before administration | After administration |
|---|---|---|
| Main agent group | 9.30 ± 0.67 | 10.75 ± 0.19 |
| (23 people) | | [10.35, 11.14] |

Before administration: mem ± standard deviation,
After administration: adjusted average value + standard error
[ ] 95% confidence interval of both sides

### Example 2: Administration of Drugs and other Iron Supplementation with Compound 1

Investigators can prescribe iron supplementation (e.g., oral iron) as needed to a patient taking Compound 1 to maintain ferritin ≥100 ng/mL and TSAT≥20%. In general, oral iron can be used for therapy. Intravenous iron use may be administered to subjects who have documented intolerance to oral iron and iron deficiency (e.g., ferritin <100 ng/mL and/or TSAT <20%). Discontinuation of IV iron may be required once the subject is no longer iron deficient (ferritin 100 ng/mL and TSAT≥20%).

As described herein, iron-containing compositions can cause the absorption and bioavailability of Compound 1 to decrease when simultaneously administered. To avoid or minimize these effects, such iron-containing compositions should be given at least 2 hours before and/or after taking Compound 1. Without being bound by theory, it is believed that Compound 1 and iron form a chelate in the digestive tract and suppress absorption.

Ferrous Sulfate Tablet on the Pharmacokinetics of Compound 1 in Healthy Male Subjects

A phase I, randomized, two-period, open-label, single-dose study was conducted to assess the bioavailability of Compound 1 co-administered with ferrous sulfate alone in 10 healthy male subjects. The pharmacokinetic (pK) parameters of Compound 1 administered alone and in combination with ferrous sulfate, monitor the safety and tolerability of single dose Compound 1 were assessed. The pharmacokinetic parameters of Compound 1-O-glucuronide and Compound 1-acyl-glucuronide in plasma were also characterized.

The study population consisted of subjects between 18 years and 55 years of age, body mass index (BMI) between 18 and 30 kg/m² at the Screening visit, and glomerular filtration rate (eGFR) of ≥65 mL/min/1.73 m² at the Screening visit, who were healthy adult males without any history of significant medical conditions.

The maximum duration of study participation for each subject was approximately 27 days including 4 periods:
Screening Period: 21 days
Confinement Period 1: 24 hours
Washout Period: 4 days
Confinement Period 2: 24 hours

Following a screening period of 21 days, subjects who meet all inclusion and no exclusion criteria were randomized in one of two treatment sequences (Compound 1 followed by Compound 1+Iron, or Compound 1+Iron followed by Compound 1).

Criteria for Evaluation

Pharmacokinetics. Blood samples for the measurement of Compound 1 and the two glucuronide metabolites (Compound 1-O-glucuronide and Compound 1-acyl-glucuronide) in plasma were collected relative to each single dose as follows: 0 hour (pre-dose), 0.5, 1, 1.5, 2, 3, 4, 5, 6, 8, 12, 16, and 24 hours after study drug administration. The following PK parameters were estimated from the plasma concentration-time profiles based upon actual collections times for Compound 1, Compound 1-O-glucuronide and Compound 1-acyl-glucuronide: Cₘₐₓ, AUC₀₋ₜ, AUC_{0-inf}, Tₘₐₓ, AUC_% extrap, λz, t_{1/2}, CL/F (parent) and V₂/F (parent). The ratio of metabolite to parent was calculated for each glucuronide metabolite for AUC₍₀₋ₜ₎; and AUC_{(0-inf)} and Cₘₐₓ.

Comparative Bioavailability. Using MIXED procedures in SAS, ANOVA was performed on In-transformed AUC₍₀₋ₜ₎; and AUC_{(0-inf)} and Cₘₐₓ for Compound 1. Factors incorporated in the model included: Sequence, Subject (Sequence), Period, and Treatment. The ratio of means (Compound 1+Iron/Compound 1) and a 90% confidence interval for the ratio of means based on least-squares means from the ANOVA of the In-transformed data, was calculated for AUC₍₀₋ₜ₎; and AUC_{(0-inf)} and Cₘₐₓ for Compound 1. In addition, a 90% geometric confidence interval (converted back to the original units) of the ratio (Compound 1/Compound 1+Iron) was presented for AUC₍₀₋ₜ₎; and AUC_{(0-inf)} and Cₘₐₓ for Compound 1.

Safety and Tolerability. Safety analyses included adverse events (AEs), clinical laboratory tests, vital signs and physical examinations.

Treatment of Subjects. Each subject was randomized to one of two treatment sequences (Compound 1 followed by Compound 1+Iron, or Compound 1+Iron followed by Compound 1) according to randomization schedule prepared prior to the start of the study. Each subject received a single 450 mg dose of Compound 1 (administered orally as three 150 mg tablets) during one study period and a single 450 mg dose of Compound 1 (administered orally as three 150 mg tablets) plus 325 mg of ferrous sulfate (one tablet, 65 mg elemental iron) during the other study period. All subjects are dosed on the same day for Day 1 of Confinement Period 1, and are crossed over to the alternate treatment and are dosed on the same day for Day 1 of Confinement Period 2. Both treatments are administered in a fasted state, and a four-day Washout Period separates each Confinement Period.

Data Analysis

Pharmacokinetic Results. Concentration data for all 10 subjects who received study drug were included in the PK and statistical analysis. Plasma Compound 1 PK parameters and statistical analysis results are summarized in Table 6 below.

TABLE 6
Pharmacokinetic Parameters

| | Least Squares (LS) | | | 90% Confidence |
|---|---|---|---|---|
| | Geometric Means | | Geometric | Interval |
| PK | | Compound | Mean | for Ratio |
| Parameter | Compound 1 | 1 + Iron | Ratio^{a} | of LS Means |
| AUC₍₀₋ₜ₎ | 253 | 117 | 46.0 | 36.7-57.6 |
| (h · μg/mL) | | | | |
| AUC_{(0-inf)} | 263 | 122 | 46.3 | 37.1-57.8 |
| (h · μg/mL) | | | | |
| Cₘₐₓ | 45.3 | 22.3 | 49.3 | 37.8-64.4 |
| (μg/mL) | | | | |

Abbreviations: h = hour; PK = pharmacokinetic.
^{a}Geometric Mean Ratio: Compound 1 + Iron/Compound 1. Ratios and confidence intervals are expressed as percentages.

Mean Compound 1 plasma AUC (AUC₀₋ₜ and AUC_{0-inf}) was approximately 54% lower following Compound 1+Iron as compared to Compound 1 administered alone. Cₘₐₓ for the combination treatment was approximately 51% lower than Cₘₐₓ for Compound 1 administered alone. Median Compound 1 Tₘₐₓ values were slightly prolonged. from 3.0 to 4.0 hours, when iron was co-administered with Compound 1, although the range of values were identical between the two treatments (1.5 to 5.0 hours).

Plasma Compound 1-O-glucuronide and Compound 1-acyl-glucuronide PK parameters are summarized in Table 7 below.

TABLE 7
Plasma Compound 1-O-glucuronide and Compound 1-acyl-glucuronide

| | Compound 1-O-glucuronide | | Compound 1-Acyl-glucuronide | |
|---|---|---|---|---|
| | N = 10 | | N = 10 | |
| PK | | Compound | | Compound |
| Parameter | Compound 1 | 1 + Iron | Compound 1 | 1 + Iron |
| AUC₍₀₋ₜ₎ | 42.8 | 18.6 | 0.208 | 0.0864 |
| (h · μg/mL) | (10.8) | (8.21) | (0.0649) | (0.0391) |
| AUC_{(0-inf)} | 44.5 | 19.7 | NC | NC |
| (h · μg/mL) | (11.4) | (9.03) | | |
| Cₘₐₓ | 6.07 | 2.83 | 0.0826 | 0.0322 |
| (μg/mL) | (1-25) | (1.10) | (0.0384) | (0.0185) |
| Tₘₐₓ | 4.00 | 5.00 | 1.75 | 3.00 |
| (h)^{a} | (3.00-6.00) | (3.00-6.00) | (0.50-5.00) | (1.50-5.00) |
| Metabolite | 0.104 | 0.0935 | 0.00543 | 0.000458 |
| Ratio | (0.0120) | (0.0139) | (0.000221) | (0.000209) |
| AUC₍₀₋ₜ₎ | | | | |
| Metabolite | 0.104 | 0.0943 | NC | NC |
| Ratio | (0.0121) | (0.0138) | | |
| AUC_{(0-inf)} | | | | |
| Metabolite | 0.0848 | 0.0748 | 0.00112 | 0.000909 |
| Ratio Cₘₐₓ | (0.0173) | (0.0117) | (0.000478) | (0.000504) |

Note:
Metabolite Ratio = Metabolite/Parent, accounting for differences in molecular weight.
Abbreviations: h = hour; N = number of subjects; NC = not calculated, parameter is not estimable for more than ⅓ of the subjects; PK = pharmacokinetic; SD = standard deviation.
^{a}Median (range).

Mean plasma AUC (AUC₍₀₋ₜ₎ and AUC_{(0_inf)} was reduced >50% for the two glucuronide metabolites when Compound 1 is administered with ferrous sulfate (iron) as compared to Compound 1 alone. Median Tₘₐₓ values for Compound 1-Oglucuronide and Compound 1-acyl-glucuronide concentrations are slightly delayed, from 4.0 to 5.0 hours, and 1.75 to 3.0 hours, respectively, when iron is co-administered with Compound 1.

Metabolite to parent drug ratios (based on AUC and Cₘₐₓ) were similar between the two treatments for both Compound 1-O-glucuronide and Compound 1-acyl-glucuronide.

Safety Results. There were no AEs, SAEs, or deaths reported during the study.

Sodium ferrous citrate, Citric acid ferric hydrate, Sucroferric oxyhydroxide, Ferrous sulfate (slow release) on the Pharmacokinetics of Compound 1 in Healthy Male Subjects

A phase 1, single-dose, open-label, randomized, crossover study was conducted in 61 healthy adult male subjects to evaluate the pharmacokinetics and safety of Compound 1 when Compound 1 and oral iron or iron-containing phosphate binders were co-administered.

The study included 3 periods as follow:
Screening: performed within 4 weeks before the day of administration of the study drug.
Evaluation period: Cohort 1 was confined to the study site for 10-night, 11-day. Cohort 2 and Cohort 3 were confined to the study site for 7 nights and 8 days.
Post-administration tests: performed 3 days after administration of the last dose of the study drug.

Following the screening period, subjects who meet all inclusion and no exclusion criteria described below were randomized into 3 cohorts in the subsequent evaluation period.

Inclusion criteria:
1. Men aged no younger than 20 years and no older than 45 years at the time of informed consent.
2. Japanese (both parents and both grandparents must be Japanese).
3. Persons whom the investigator/subinvestigator deem to be suitable as a subject via the screening tests, and the interview and examinations on Day −1 (day before administration of the study drug) and Day 1 (day of administration of the study drug).

Exclusion criteria:
1. Persons with a current medical history or treatment history (drug treatment, in-patient treatment, etc.) as follows.
   1) Heart: Angina pectoris, arrhythmia, myocardial infarction, heart failure, or other heart disease
   2) Liver: Liver disease with decreased liver function
   3) Kidney: Kidney disease with decreased kidney function
   4) Gastrointestinal system: Gastric ulcer, pancreatitis, or other gastrointestinal disease (excluding, however, persons with no recurrence for at least 5 years on drug therapy alone)
   5) Respiratory system: Pulmonary tuberculosis, obstructive pulmonary disease, or other respiratory disease
   6) Nervous system: Diseases accompanied by sensory disorder or other neurological disorder
   7) Hematopoietic function: Blood disease accompanies by findings of anemia with pronounced deviations from reference values
   8) Endocrine function: Endocrine disease that is deemed to have a pronounced effect on endocrine function such as thyroid, parathyroid, or pituitary function
   9) Other: Malignant tumor
2. Persons with signs of heart disease at screening (for example, QTcF interval of 450 msec or greater in 12-lead ECG findings).
3. Persons with a history or current symptoms of drug or alcohol dependence.
4. Persons who the investigator/subinvestigator believe cannot adhere to the prohibitions during the confinement period.
5. Persons who have previously used Compound 1.
6. Persons with a history or current symptoms of drug or food allergy.
7. Persons with, at screening, BMI less than 18.5 kg/m2 or greater than 25.0 kg/m2. Alternatively, persons with body weight less than 50.0 kg (in BMI calculations, the second decimal place is rounded off).
8. Persons who have donated blood components with 2 weeks before informed consent. Alternatively, persons who have donated blood or had blood drawn in an amount of 400 mL or more within 12 weeks or 200 mL or more within 4 weeks before informed consent.
9. Persons who have donated or had blood drawn in an amount of 800 mL or more within 1 year before informed consent.
10. Persons with a history of surgical procedures that are known to have an effect on gastrointestinal drug absorption (excluding appendicectomy and hernia operations).
11. Persons with a positive result for HBs antigen, serological test for syphilis, HCV antibody, or HIV antigen or antibody at screening.
12. Persons who are unwilling to consent to use contraception from the beginning of the study period to 90 days following the final dose of the study drug.
13. Persons who have participated in another clinical study and received study drugs within 12 weeks prior to informed consent, or within 5 times the half-life of the study drug (whichever is longer).
14. Persons who used any medications other than the study drug to be administered in this study or other therapies within 7 days before the start of administration of the study drug.
15. Persons who consumed any supplements within 7 days before the start of administration of the study drug.
16. Persons who consumed grapefruit or other citrus fruits, apples, or processed foods containing any of these within 7 days before the start of administration of the study drug.
17. Persons who consumed health foods containing St. John's wort (Japanese name, seiyo-otogiri-so) within 2 weeks before the start of administration of the study drug.
18. Other persons judged by investigators/subinvestigators to be inappropriate as a subject in this study.

Criteria for Evaluation

Pharmacokinetics. Blood samples for measurement of the plasma concentration of unchanged Compound 1 were collected as follow in all three Cohorts: immediately before administration of the study drug, and at 0.5, 1, 1.5, 2, 3, 4, 5, 6, 8, 12, 16, and 24 hours after administration of the study drug. For each evaluation period, the plasma concentration of unchanged Compound 1 will be assayed, and the following parameters calculated by non-compartmental analysis: AUC_{0-inf}, Cₘₐₓ, AUC₀₋ₗₐₛₜ, Kel, and t_{1/2}.

Safety. Safety analyses included adverse events (AEs), adverse drug reaction (ADRs), general laboratory tests, vital signs and standard 12-lead ECG (electrocardiogram).

Treatment of Subjects

Subjects were randomized into 3 cohorts as described below in the evaluation period.

Cohort 1 (Administration after meal)

In Cohort 1, 21 subjects were further randomized into 3 groups (7 subjects per group) and the evaluation period was divided into 3 periods during the study:

| | Period 1 | Period 2 | Period 3 |
|---|---|---|---|
| | (Day 1) | (Day 4) | (Day 7) |
| Group 1 | Vadadustat Tablets | Vadadustat Tablets | Vadadustat Tablets |
| | | and Sodium | and Ferric |
| | | Ferrous Citrate | Citrate Hydrate |
| Group 2 | Vadadustat Tablets | Vadadustat Tablets | Vadadustat Tablets |
| | and Ferric Citrate | | and Sodium |
| | Hydrate | | Ferrous Citrate |
| Group 3 | Vadadustat Tablets | Vadadustat Tablets | Vadadustat Tablets |
| | and Sodium Ferrous | and Ferric | |
| | Citrate | Citrate Hydrate | |

Administration of vadadustat tablets alone: after fasting for at least 10 hours, subjects would eat breakfast over a period of 10 minutes, then immediately after finishing the meal (5 minutes after finishing the meal), 1 vadadustat tablet (150 mg) would be taken orally with 200 mL of water (extra water may be drunk if more is needed when taking the tablet, and the extra volume would be recorded). After breakfast, subjects would fast until the end of the collection of blood samples at 4 hours after administration of the study drug. Subjects would eat lunch over a period of 20 minutes after blood samples were collected at 4 hours after administration of the study drug. After lunch, subjects would fast until dinner, when they would eat dinner over a period of 20 minutes at 10 hours after administration of the study drug. Subjects would fast from dinner until breakfast the next morning.

Administration of vadadustat tablets and sodium ferrous citrate: after fasting for at least 10 hours, subjects would eat breakfast over a period of 10 minutes, then immediately after finishing the meal (5 minutes after finishing the meal), 1 vadadustat tablet (150 mg) and 4 tablets of sodium ferrous citrate (containing 200 mg of iron) would be taken orally with 200 mL of water (extra water may be drunk if more is needed when taking the tablets, and the extra volume would be recorded). After breakfast, subjects would fast until the end of the collection of blood samples at 4 hours after administration of the study drug. Subjects would eat lunch over a period of 20 minutes after blood samples were collected at 4 hours after administration of the study drug. After lunch, subjects would fast until dinner, when they would eat dinner over a period of 20 minutes at 10 hours after administration of the study drug. Subjects would fast from dinner until breakfast the next morning.

Administration of vadadustat tablets and ferric citrate hydrate: after fasting for at least 10 hours, subjects would eat breakfast over a period of 10 minutes, then immediately after finishing the meal (5 minutes after finishing the meal), 1 vadadustat tablet (150 mg) and 8 tablets of ferric citrate hydrate (containing 2000 mg of ferric citrate) would be taken orally with 200 mL of water (extra water may be drunk if more is needed when taking the tablets, and the extra volume will be recorded). After breakfast, subjects would fast until the end of the collection of blood samples at 4 hours after administration of the study drug. After collection of blood samples at 4 hours after administration of the study drug, subjects would eat lunch over a period of 20 minutes, then immediately after finishing the meal, 8 tablets of ferric citrate hydrate (containing 2000 mg of ferric citrate) would be taken orally with 200 mL of water (extra water may be drunk if more is needed when taking the tablet, and the extra volume would be recorded). Subjects would fast from lunch to before dinner, and at 10 hours after administration of the study drug, subjects will eat dinner over a period of 20 minutes, then immediately after finishing the meal, 8 tablets of ferric citrate hydrate (containing 2000 mg of ferric citrate) would be taken orally with 200 mL of water (extra water may be drunk if more is needed when taking the tablets, and the extra volume would be recorded). Subjects would fast from dinner until breakfast the next morning.

Cohort 2 (Administration Before Meal)

In Cohort 2, 20 subjects were further randomized into 2 groups (10 subjects per group) and the evaluation period was divided into 2 periods during the study:

| | Period 1 | Period 2 |
|---|---|---|
| | (Day 1) | (Day 4) |
| Group 4 | Vadadustat Tablets | Vadadustat Tablets |
| | | and Sucroferric |
| | | Oxyhydroxide |
| Group 5 | Vadadustat Tablets | Vadadustat Tablets |
| | and Sucroferric | |
| | Oxyhydroxide | |

Administration of vadadustat tablets alone: after fasting for at least 10 hours and immediately before eating breakfast (5 minutes before starting the meal), 1 vadadustat tablet (150 mg) will be taken orally with 200 mL of water, then breakfast would be eaten over a period of 10 minutes (extra water may be drunk if more is needed when taking the tablet, and the extra volume would be recorded). After breakfast, subjects would fast until the end of the collection of blood samples at 4 hours after administration of the study drug. Subjects would eat lunch over a period of 20 minutes after blood samples were collected at 4 hours after administration of the study drug. After lunch, subjects would fast until dinner, when they would eat dinner over a period of 20 minutes at 10 hours after administration of the study drug. Subjects would fast from dinner until breakfast the next morning.

Administration of vadadustat tablets and sucroferric oxyhydroxide: after fasting for at least 10 hours and immediately before eating breakfast (5 minutes before starting the meal), subjects would chew and swallow 2 tablets of sucroferric oxyhydroxide (containing 1000 mg of iron), then 1 vadadustat tablet (150 mg) would be taken orally with 200 mL of water, then breakfast would be eaten over a period of 10 minutes (extra water may be drunk if more is needed when taking the tablets, and the extra volume would be recorded). After breakfast, subjects would fast until the end of the collection of blood samples at 4 hours after administration of the study drug. After blood samples were collected at 4 hours after administration of the study drug and immediately before lunch, subjects would chew and swallow 2 tablets of sucroferric oxyhydroxide (containing 1000 mg of iron), then eat lunch over a period of 20 minutes. After lunch, subjects will fast until dinner, and immediately before dinner at 10 hours after administration of the study drug, chew and swallow 2 tablets of sucroferric oxyhydroxide (containing 1000 mg of iron), then eat dinner over a period of 20 minutes. Subjects would fast from dinner until breakfast the next morning.

Cohort 3 (Administration in Fasting)

In Cohort 3, 20 subjects were further randomized into 2 groups (10 subjects per group) and the evaluation period was divided into 2 periods during the study:

| | Period 1 | Period 2 |
|---|---|---|
| | (Day 1) | (Day 4) |
| Group 6 | Vadadustat Tablets | Vadadustat Tablets |
| | | and Dried Ferrous |
| | | Sulfate (Slow |
| | | Release) |
| Group 7 | Vadadustat Tablets | Vadadustat Tablets |
| | and Dried Ferrous | |
| | Sulfate (Slow | |
| | Release) | |

Administration of vadadustat tablets alone: after fasting for at least 10 hours, 1 vadadustat tablet (150 mg) would be taken orally with 200 mL of water (extra water may be drunk if more is needed when taking the tablet, and the extra volume would be recorded). Subjects would fast until the end of the collection of blood samples at 4 hours after administration of the study drug. Subjects would eat lunch over a period of 20 minutes after blood samples were collected at 4 hours after administration of the study drug. After lunch, subjects would fast until dinner, when they would eat dinner over a period of 20 minutes at 10 hours after administration of the study drug. Subjects would fast from dinner until breakfast the next morning.

Administration of vadadustat tablets and dried ferrous sulfate: after fasting for at least 10 hours, 1 vadadustat tablet (150 mg) and 2 tablets of dried ferrous sulfate (containing 210 mg of iron) would be taken orally with 200 mL of water (extra water may be drunk if more is needed when taking the tablets, and the extra volume would be recorded). Subjects would fast until the end of the collection of blood samples at 4 hours after administration of the study drug. Subjects would eat lunch over a period of 20 minutes after blood samples were collected at 4 hours after administration of the study drug. After lunch, subjects would fast until dinner, when they would eat dinner over a period of 20 minutes at 10 hours after administration of the study drug. Subjects would fast from dinner until breakfast the next morning.

Data Analysis

Pharmcokinetic Results for Cohort 1.

Concentration data for Cohort 1 subjects who received study drug were included in the PK and statistical analysis. Plasma Compound 1 PK parameters and statistical analysis results are summarized in Table 8 below.

TABLE 8
Pharmacokinetic Parameters (Cohort 1 subjects)

| | | Least Squares (LS) | | | 90% |
|---|---|---|---|---|---|
| | | Geometric Means | | | Confidence |
| | | | Compound | Geometric | Interval for |
| | PK | Compound 1 | 1 + Iron | Mean | Ratio of LS |
| Treatment | Parameter | (21 subjects) | (20 subjects) | Ratio^{a} | Means |
| with | Cₘₐₓ | 14.30 | 6.96 | 48.66 | 40.55-58.39 |
| sodium | (μg/mL) | | | | |
| ferrous | AUC_{(0-inf)} | 104.02 | 46.59 | 44.79 | 38.14-52.60 |
| citrate | (h · μg/mL) | | | | |
| | AUC₍₀₋ₗₐₛₜ₎ | 99.22 | 44.41 | 44.76 | 37.99-52.74 |
| | (h · μg/mL) | | | | |
| | Kel (/h) | 0.13 | 0.13 | 99.76 | 94.03-105.82 |
| | t_{1/2} (h) | 5.32 | 5.34 | 100.25 | 94.50-106.34 |
| with | Cₘₐₓ | 14.30 | 5.18 | 36.25 | 30.21-43.51 |
| ferric | (μg/mL) | | | | |
| citrate | AUC_{(0-inf)} | 104.02 | 32.35 | 31.10 | 26.49-36.53 |
| hydrate | (h · μg/mL) | | | | |
| | AUC₍₀₋ₗₐₛₜ₎ | 99.22 | 31.14 | 31.39 | 26.64-36.98 |
| | (h · μg/mL) | | | | |
| | Kel (/h) | 0.13 | 0.16 | 125.00 | 117.83-132.61 |
| | t_{1/2} (h) | 5.32 | 4.26 | 80.00 | 75.41-84.87 |

The geometric mean ratios of AUC_{(0-inf)} and Cₘₐₓ for each treatment were less than 50%, and the 90% confidence intervals for AUC_{(0-inf)} and Cₘₐₓ were outside the limits for bioequivalence of 80.0-125%.

Pharmacokinetic Results for Cohort 2.

Concentration data for all 20 Cohort 2 subjects who received study drug were included in the PK and statistical analysis. Plasma Compound 1 PK parameters and statistical analysis results are summarized in Table 9 below.

TABLE 9
Pharmacokinetic Parameters (Cohort 2 subjects)

| | | Least Squares (LS) | | | 90% |
|---|---|---|---|---|---|
| | | Geometric Means | | | Confidence |
| | | | Compound | Geometric | Interval for |
| | PK | Compound 1 | 1 + Iron | Mean | Ratio of LS |
| Treatment | Parameter | (20 subjects) | (20 subjects) | Ratio^{a} | Means |
| with | Cₘₐₓ | 15.20 | 8.81 | 57.95 | 49.91-67.28 |
| sucroferric | (μg/mL) | | | | |
| oxyhydroxide | AUC_{(0-inf)} | 99.47 | 45.74 | 45.99 | 40.79-51.85 |
| | (h · μg/mL) | | | | |
| | AUC₍₀₋ₗₐₛₜ₎ | 95.68 | 44.54 | 46.56 | 41.24-52.55 |
| | (h · μg/mL) | | | | |
| | Kel (/h) | 0.13 | 0.16 | 119.41 | 111.94-127.39 |
| | t_{1/2} (h) | 5.17 | 4.33 | 83.74 | 78.50-89.34 |

The geometric mean ratios of AUC_{(0-inf)} was 46% and Cₘₐₓ was 58%. The 90% confidence intervals for AUC_{(0-inf)} and Cₘₐₓ were outside the limits for bioequivalence of 80.0 -125%.

Pharmacokinetic Results for Cohort 3.

Concentration data for all 20 Cohort 3 subjects who received study drug were included in the PK and statistical analysis. Plasma Compound 1 PK parameters and statistical analysis results are summarized in Table 10 below.

TABLE 10
Pharmacokinetic Parameters (Cohort 3 subjects)

| | | Least Squares (LS) | | | 90% |
|---|---|---|---|---|---|
| | | Geometric Means | | | Confidence |
| | | | Compound | Geometric | Interval for |
| | PK | Compound 1 | 1 + Iron | Mean | Ratio of LS |
| Treatment | Parameter | (20 subjects) | (20 subjects) | Ratio^{a} | Means |
| with dried | Cₘₐₓ | 26.49 | 2.14 | 8.09 | 6.20-10.57 |
| ferrous | (μg/mL) | | | | |
| sulfate | AUC_{(0-inf)} | 128.00 | 13.20 | 10.31 | 8.03-13.25 |
| (slow | (h · μg/mL) | | | | |
| release) | AUC₍₀₋ₗₐₛₜ₎ | 123.79 | 12.03 | 9.72 | 7.46-12.67 |
| | (h · μg/mL) | | | | |
| | Kel (/h) | 0.13 | 0.14 | 107.72 | 97.06-119.55 |
| | t_{1/2} (h) | 5.17 | 4.80 | 92.83 | 83.64-103.03 |

The geometric mean ratios of AUC_{(0-inf)} and Cₘₐₓ were approximately 10% and the 90% confidence intervals for AUC_{(0-inf)} and Cₘₐₓ were outside the limits for bioequivalence of 80.0-125%.

Effects of Iron-Containing Compositions (Simultaneous Administration)

Effects of simultaneous oral iron or iron-containing phosphorus adsorbent on the pharmacokinetics of Compound 1 are summarized in Table 11 below (at the time of simultaneous administration).

TABLE 11
Effects of Iron-Containing Compositions on
Pharmacokinetic Parameters of Compound 1

| | | | | Pharmacokinetic parameters | |
|---|---|---|---|---|---|
| | | | | of this drug Ratio of | |
| | | | | geometric mean value (%) | |
| | Iron- | | This | [90% confidence intercal] | |
| Concomitant | containing | | Formulation | Combined/alone | |
| Medication | Amount | | Dosage | Cₘₐₓ | AUC_{0-∞} |
| Ferrous sulfate | 65 | mg | 450 mg | 49.3 | 46.0 |
| Preparation | | | | [37.8-64.4] | [36.7-57.6] |
| Sodium ferrous | 200 | mg | 150 mg | 48.66 | 44.79 |
| citrate | | | | [40.55-58.39] | [38.14-52.60] |
| preparation | | | | | |
| Citric acid ferric | 2000 | mg^{a)} | 150 mg | 36.25 | 31.10 |
| hydrate tablet | | | | [30.21-43.51] | [26.49-36.33] |
| Sucroferric | 1000 | mg | 150 mg | 57.95 | 45.99 |
| oxyhydroxide | | | | [49.91-67.28] | [40.79-51.85] |
| chewable tablets | | | | | |
| Iron sulfate slow | 210 | mg | 150 mg | 8.09 | 10.31 |
| release tablet | | | | [6.20-10.57] | [8.03-13.25] |

^{a)}Dosage as ferric citrate

Thus, simultaneous administration of oral iron can reduce the bioavailability of Compound 1. The reduction in bioavailability may be due to formation of a chelate complex between Compound 1 and iron. These affects can be reduced and/or minimized by administering oral iron and Compound 1 at least about two hours apart.

### Example 3: Administration of Calcium-Containing Composition with Compound 1

Investigators can prescribe calcium-containing composition (e.g., as phosphate binders) to a patient taking Compound 1. For example, such calcium-containing composition can be administered to maintain serum phosphorus at a target level (e.g., below 5.5 mg/dL).

A phase I, open-label, fixed-sequence study was conducted in healthy adult subjects to assess the effect of 1) a single oral dose of calcium acetate on the pharmacokinetics of a single oral dose of vadadustat.

The study included screening (28 days maximum), 9-day treatment period, and a 14-day follow-up period post last dose.

Following the screening period, subjects who meet all inclusion and no exclusion criteria described below were randomized in the subsequent treatment period. A total of 18 subjects were enrolled in the study.

Inclusion criteria:
1. Male or female between 18 and 55 years of age, inclusive, at time of informed consent.
   a. Female subjects of childbearing potential must be non-lactating, not pregnant as confirmed by a negative serum pregnancy test at screening and Day −1, and using, and agree to continue using, an effective method of contraception for at least of 4 weeks prior to the first dose of study drug until 30 days after the last dose of study drug.
   b. Female subjects of non-childbearing potential must be surgically sterile (e.g., hysterectomy, bilateral tubal ligation, oophorectomy) or post-menopausal (no menses for >1 year with follicle stimulating hormone [FSH]>40 U/L at screening).
   c. Female subjects of childbearing potential must agree not to donate ova during the study and for at least 30 days after the last dose of study drug.
   d. Male subjects who have not had a vasectomy for at least 6 months must agree to use an effective method of contraception during the study and until 90 days after the last dose of the study drug, and to not donate sperm during the study and for at least 90 days after the last dose of study drug.
2. Healthy per investigator judgment as documented by medical history, physical examination, vital sign assessments, 12-lead ECG, clinical laboratory assessments, and general observations.
   a. At screening, abnormalities or deviations outside the normal ranges for any clinical assessments (laboratory tests, ECG, vital signs) may be repeated once at the discretion of the investigator(s), and results that continue to be outside the normal ranges must be judged by the investigator to be not clinically significant and acceptable for study participation.
   b. On Day −1, alanine aminotransferase (ALT), aspartate aminotransferase (AST) and total bilirubin values must be within the upper limits of the normal range. All other laboratory test results that are outside the normal range on Day −1 and judged by the investigator to be not clinically significant may be repeated. Results that continue to be outside the normal range must be judged by the investigator to be not clinically significant and acceptable for study participation.
3. Body mass index (BMI) between 18.0 and 30 kg/m2, with a minimum body weight of 45 kg for females and 50 kg for males, inclusive.
4. Understands the procedures and requirements of the study and provides written informed consent and authorization for protected health information disclosure
5. Willing and able to comply with the requirements of the study protocol.

Exclusion Criteria
1. Current or past history of cardiovascular, cerebrovascular, respiratory, gastrointestinal, hematologic, renal, hepatic, immunologic, metabolic, urologic, neurologic, dermatologic, psychiatric, or other major disease, as determined by the investigator. History of cancer (except treated non-melanoma skin cancer) or history of chemotherapy use within 5 years prior to screening.
2. Any surgical or medical condition or history that, in the opinion of the investigator, may potentially alter the absorption, metabolism, or excretion of study treatment, such as, but not limited to gastric bypass surgery or gastric or duodenal ulcers.
3. Clinically significant history of dysphagia, bowel obstruction, or perforation.
4. Clinically significant history of hypercalcemia.
5. Clinically significant history of iron overload.
6. Clinically significant history of liver disease.
7. Clinically significant history of hypophosphatemia, ulcerative colitis, or gastrointestinal bleeding.
8. Contraindication to study drugs or its excipients and/or history of allergic or anaphylactic reactions.
9. Taking any of the following prohibited medications:
   a. Any prescription medication or over the counter multi-vitamin supplement, or any nonprescription products (including herbal-containing preparations but excluding acetaminophen up to 2 g daily) within 14 days prior to Day −1,
   b. Any drug known to inhibit or induce cytochrome P450 (CYP) enzymes and/or P-glycoprotein including St. John's wort (Hypericum perforatum) within 14 days or 5 half-lives (whichever is longer) prior to Day −1.
10. History of drug abuse within the previous year prior to screening or use of soft drugs (such as marijuana) within 3 months prior to the screening visit or hard drugs (such as cocaine, phencyclidine [PCP], crack, opioid derivatives including heroin, and amphetamine derivatives) within 1 year prior to screening.
11. History of regular alcohol consumption exceeding 14 drinks/week (1 drink=5 ounces [150 mL] of wine or 12 ounces [360 mL] of beer or 1.5 ounces [45 mL] of hard liquor) within 6 months of screening or alcohol abuse within 1 year prior to screening.
12. Positive drug and alcohol test at screening or on Day −1.
13. History of latent or active tuberculosis (TB) as per documented medical history. Exposure to endemic areas within 8 weeks of screening.
14. Daily use of nicotine-containing products within 6 months of screening.
15. Consumed any food or drink/beverage containing grapefruit or grapefruit juice, apple or orange juice, pomelo juice, pomegranate, pineapple, star fruit, Seville or Moro (blood) orange products, and vegetables from the mustard green family (e.g., kale, broccoli, watercress, collard greens, kohlrabi, brussels sprouts, mustard) and charbroiled meats known to modulate CYP enzyme activity and transporters within 7 days before administration of study drug.
16. Positive test results of hepatitis B surface antigen (HBsAg), or positive hepatitis C virus antibody (HCVab) test result within 3 months prior to Day −1 or at screening.
17. Positive test results for human immunodeficiency virus (HIV) antibody within 3 months prior to Day −1 or at screening.
18. Participation in another clinical trial or exposure to any investigational agent within 30 days or 5 half-lives prior to Day −1, whichever is longer.
19. Donation of plasma within 7 days prior to dosing. Donation or loss of blood (excluding volume drawn at screening) of 50 mL to 499 mL of blood within 30 days, or more than 499 mL within 56 days prior to the first dosing.
20. Received a tattoo or body piercing (including ear piercings) within 2 months prior to Day 1, and/or open wound that may result in risk of infection.
21. Having a condition that the investigator believes would interfere with his/her ability to provide written informed consent, comply with study instructions, or which might confound the interpretation of the study results or put the subject at undue risk.
22. Have previously participated in a clinical study that administered vadadustat.

Criteria for Evaluation

Pharmacokinetics. A predose PK blood sample was collected on Day 1. After each dose of vadadustat administered (Days 1, 3, 5, and 7), blood samples for PK analysis were collected at the following time points: predose and 0.5, 1, 1.5, 2, 3, 4, 6, 9, 12, 16, 24, and 48 hours after each dose of vadadustat. The 48-hour postdose sample was collected before vadadustat administration on Days 3, 5, and 7. The following parameters were calculated and analyzed: AUC_{0-∞}, AUC₀₋ₗₐₛₜ, Cₘₐₓ, Tₘₐₓ, Kel, CIB, V_{c}/F, and t_{1/2}.

Safety. Safety and tolerability were evaluated by adverse event reporting, assessment of 12-lead electrocardiograms (ECGs), vital signs, physical examination and clinical laboratory tests.

Treatment of Subjects.

Subjects were dosed approximately at the same designated time on each dosing day, where applicable. Study drugs were administered with approximately 8 ounces (240 mL) of water at room temperature in a seated position. Subjects were instructed to drink the full 8 ounces of water, and not to chew or break the tablets/gelcaps.

The 9-day treatment period was conducted as follows:
Day 1: vadadustat administered immediately after consuming breakfast.
Day 2: wash-out (no investigational product administration).
Day 3: vadadustat administered concomitantly with the requisite phosphate binder immediately after consuming breakfast.
Day 4: wash-out (no investigational product administration).
Day 5: vadadustat administered in fasted condition and the phosphate binder administered 1 hour later (breakfast must be served within 2 minutes after phosphate binder administration and consumed immediately after it is served).
Day 6: wash-out (no investigational product administration).
Day 7: phosphate binder administered immediately after consuming breakfast; vadadustat administered 2 hours after the phosphate binder was administered.
Day 8: wash-out (no investigational product administration).
Day 9: subjects discharged from the Clinical Research Unit (CRU) after the 48-hour postdose PK blood sample has been collected.

A single oral dose of vadadustat 300 mg (2×150 mg tablets) was administered on Days 1, 3, 5, and 7. Phosphate binder that is calcium acetate (1334 mg, 2×667 mg gelcaps) was administered on Days 3, 5, and 7.

Data Analysis

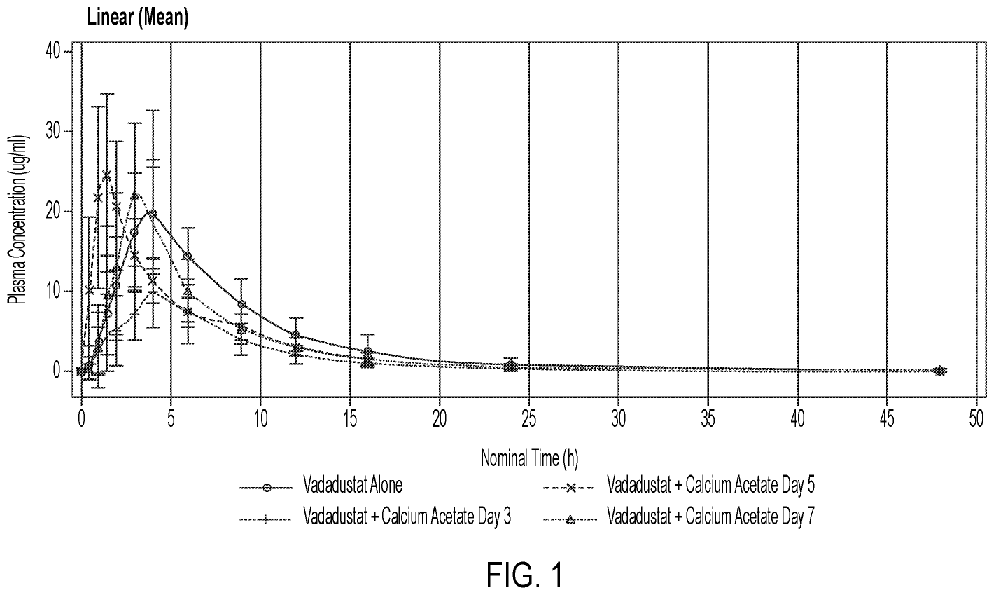

Vadadustat plasma PK parameter findings are summarized in Table 12. Plasma concentration-time profiles for vadadustat alone and when coadministered with calcium acetate are presented in FIG. 1.

TABLE 12
Summary of plasma PK parameters for vadadustat
following coadministration with calcium acetate

| | Calcium acetate + vadadustat | | | |
|---|---|---|---|---|
| | | | Vadadustat | Vadadustat |
| | | | administered 1 | administered 2 |
| | | | hour prior | hours after |
| | Vadadustat | Concomitant | to calcium | calcium |
| | alone^{a} | administration^{b} | acetate^{c} | acetate^{d} |
| PK parameters | (n = 18) | (n = 18) | (n = 17) | (n = 18) |
| AUC₀₋ₗₐₛₜ | 163 ± 45.7 | 77.4 ± 31.75 | 130 ± 36.2 | 132 ± 44.3 |
| (μg · h/mL) | | | | |
| AUC_{0-∞} | 166 ± 45.4 | 79.6 ± 32.7 | 133 ± 36.8 | 135 ± 44.3 |
| (μg · h/mL) | | | | |
| Cₘₐₓ | 21.9 ± 5.61 | 11.3 ± 4.39 | 25.0 ± 9.56 | 24.6 ± 7.13 |
| (μg/mL) | | | | |
| Tₘₐₓ | 3.93 | 4.00 | 1.44 | 2.97 |
| (h) | (1.45, 6.02) | (1.44, 5.99) | (0.93, 3.95) | (1.47, 3.95) |
| Elimination | 4.75 ± 1.20 | 4.58 ± 0.90 | 4.47 ± 0.75 | 4.84 ± 1.03 |
| half-life | | | | |
| (t_{1/2}; h) | | | | |
| Clearance | 1.91 ± 0.42 | 4.64 ± 2.47 | 2.42 ± 0.63 | 2.53 ± 1.12 |
| (Cl_{B}; L/h) | | | | |
| Volume of | 12.6 ± 2.41 | 29.8 ± 14.62 | 15.3 ± 3.25 | 17.8 ± 9.99 |
| distribution | | | | |
| (V_{c}; L) | | | | |

^{a}Vadadustat 300 mg administered immediately after breakfast.
^{b}Vadadustat 300 mg administered concomitantly with calcium acetate immediately after breakfast.
^{c}Vadadustat 300 mg administered under fasting conditions and calcium acetate administered 1 hour later, followed immediately (within 2 minutes) by breakfast.
^{d}Calcium acetate followed immediately by breakfast; vadadustat 300 mg administered 2 hours later.

Values are represented as mean±SD, expect for Tₘₐₓ, presented as median (minimum, maximum).

AUC_{0-∞}, area under the plasma concentration-time curve from dosing (time 0) to infinity; AUC₀₋ₗₐₛₜ, area under the plasma concentration-time curve from dosing (time 0) to last quantifiable concentration; Cₘₐₓ, maximum observed plasma concentration; PK, pharmacokinetics; SD, standard deviation; Tₘₐₓ, time to maximum observed plasma concentration; V_{c}, apparent volume of central component of pharmacokinetic model.

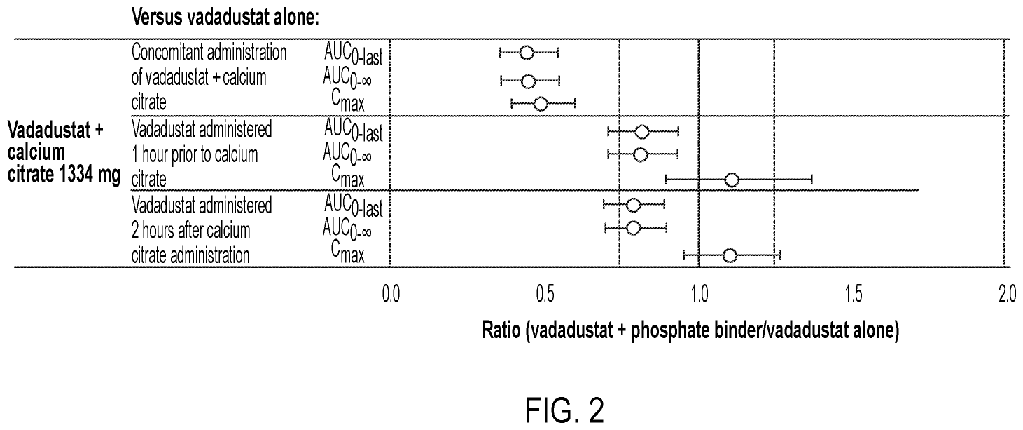

Concomitant administration of calcium acetate with vadadustat reduced vadadustat AUC₀₋ₗₐₛₜ, AUC_{0-∞} and Cₘₐₓ compared with administering vadadustat alone, whereas Tₘₐₓ and t_{1/2} remained similar and Cl_{B} and V_{c} increased (Table 12 and FIG. 1). When vadadustat was administered concurrently with calcium acetate, the geometric LS mean AUC_{0-∞}. was relatively lower at 72.44 μg·h/mL, with a geometric LS mean ratio of 45.00% (90% Cl, 36.67-55.24) to vadadustat alone. ANOVA (Analysis of variance) indicated that concomitant administration of phosphate binders with vadadustat reduced vadadustat AUC₀₋ₗₐₛₜ and AUC_{0-∞} by about 55% and Cₘₐₓ by about 51% (FIG. 2). However, when vadadustat was administered 1 hour prior to or 2 hours after calcium acetate, changes in vadadustat exposure were within the prespecified no-effect boundaries of 80% to 125%. This suggests that the DDI (drug-drug interaction) with phosphate binders such as calcium acetate can be overcome by administering vadadustat immediately upon waking, prior to breakfast, or 2 hours after administering a phosphate binder (which can continue to be administered with meals).

### Example 4: Effects of Second Drugs—Rosuvastatin, Atorvastatin, Simvastatin, Furosemide

A general overview of the effects of Compound 1 on the pharmacokinetics of a second drug is provided in Table 13.

TABLE 13
Effects of Compound 1 on Pharmacokinetic Parameters of Second Drugs

| | | | Pharmacokinetic parameters | |
|---|---|---|---|---|
| | | | of second drugs Ratio of | |
| | Concomitant | | geometric mean value (%) | |
| | Second | This | [90% confidence intercal] | |
| Concomitant | Drug | Formulation | Combined/alone | |
| Medication | Dosage | Dosage | Cₘₐₓ | AUC_{0-∞} |
| Rosuvastatin | 20 mg | 600 mg | 274.80 | 246.86 |
| | | | [246.28-306.62] | [227.08-268.36] |
| Atorvastatin | 40 mg | 600 mg | Atorvastatin | |
| | | | 100.45 | 142.05 |
| | | | [85.30-118.30] | [135.42-149.00] |
| | | | o-hydroxy atorvastatin | |
| | | | 91.20 | 112.01 |
| | | | [80.47-103.36] | [106.91-117.36] |
| | | | p-hydroxy atorvastatin | |
| | | | 230.48 | 167.57 |
| | | | [192.41-276.08] | [155.95-180.06] |
| Simvastatin | 20 mg | 600 mg | Simvastatin | |
| | | | 123.15 | 194.56 |
| | | | [104.55-145.05] | [169.77-222.97] |
| | | | β-hydroxysimvastatin acid | |
| | | | 291.84 | 246.21 |
| | | | [260.40-327.07] | [218.73-277.15] |
| Furosemide | 40 mg | 600 mg | 171.25 | 209.21 |
| | | | [136.63-214.66] | [187.07-233.97] |

Thus, the systemic exposure to the second drug, such as rosuvastatin, atorvastatin, simvastatin, or furosemide, is increased when said drugs were administered with Compound 1 compared to when said drugs were administered alone. The increase in exposure may be due to, for example, the BCRP (breast cancer resistance protein) and/or OAT3 (organic anion transporter 3) inhibitory action of Compound 1. These affects can be reduced and/or minimized by adjusting (e.g., decreasing) the amount of the second drug (e.g., rosuvastatin, atorvastatin, simvastatin, furosemide, or a drug as described herein) co-administered with Compound 1, compared to the amount when administered alone or in monotherapy.

Exemplary methods of modulating such drug-drug interactions with Compound 1 are described herein.

### Example 4a: Administration of Statin Drugs or Sulfasalazine with Compound 1

Investigators may prescribe a second drug as needed to a patient taking Compound 1 for treating or preventing a disease or condition in the patient. However, significant challenges may arise when a patient receives multiple therapeutic agents, and the therapeutic agents may interact with each other in the patient's body in a way that can adversely impact the intended therapeutic effect.

A phase I, randomized, open-label study was conducted to evaluate the potential for interaction of Compound 1 with rosuvastatin, sulfasalazine, pravastatin, atorvastatin, and simvastatin in 134 healthy male and female subjects. The pharmacokinetic (pK) parameters and safety of said drugs administered alone and in combination with Compound 1 were characterized.

The study included 3 periods as follow:
Screening: performed within 28 days prior to dosing on Day 1.
Treatment period: Subjects received rosuvastatin, sulfasalazine, pravastatin, atorvastatin, or simvastatin alone, followed by in combination with Compound 1.
Follow up period: Subjects were followed for up to 30 days (±2 days) post last dose of study drug.

Following the screening period, subjects who meet all inclusion and no exclusion criteria described below were randomized in the subsequent treatment period.

Inclusion criteria:
1. Male or female between 18 and 55 years of age, inclusive, at time of informed consent.
   a. Female subjects of childbearing potential must be non-lactating, not pregnant as confirmed by a negative serum pregnancy test at Screening and Day −1, and using, and agree to continue using, an effective method of contraception for at least of 4 weeks prior to first study drug administration until 30 days after the last dose of study drug.
   b. Female subjects of non-childbearing potential must be surgically sterile (e.g.,
   c. hysterectomy, bilateral tubal ligation, oophorectomy) or post-menopausal (no menses for >1 year with follicle stimulating hormone >40 U/L at Screening).
   d. Female subjects of childbearing potential must not donate ova during the study and for at least 30 days after the last dose of study drug.
   e. Male subjects who have not had a vasectomy must agree to use an effective method of contraception for at least 4 weeks prior to first study drug administration until 90 days after the last dose of the study drug, and to not donate sperm during the study and for at least 90 days after the last dose of study drug.
2. Healthy per investigator judgment as documented by medical history, physical examination, vital sign assessments, 12-lead ECG, clinical laboratory assessments, and general observations.
   a. At Screening, abnormalities or deviations outside the normal ranges for any clinical assessments (laboratory tests, ECG, vital signs) may be repeated once at the discretion of the investigator(s), and results that continue to be outside the normal ranges must be judged by the investigator to be not clinically significant and acceptable for study participation.
   b. On Day −1, alanine aminotransferase (ALT), aspartate aminotransferase (AST) and total bilirubin values must be within the upper limits of the normal range. All other laboratory test results that are outside the normal range on Day −1 and judged by the investigator to be not clinically significant may be repeated. Results that continue to be outside the normal range must be judged by the investigator to be not clinically significant and acceptable for study participation.
3. Body mass index between 18.0 and 30.0 kg/m², with a minimum body weight of 45 kg for females and 50 kg for males, inclusive.
4. Understands the procedures and requirements of the study and provides written informed consent and authorization for protected health information disclosure.
5. Willing and able to comply with the requirements of the study protocol.

Exclusion criteria:
1. Current or past clinically significant history of cardiovascular, cerebrovascular, pulmonary, gastrointestinal, hematologic, renal, hepatic, immunologic, metabolic, urologic, neurologic, dermatologic, psychiatric, or other major disease, as determined by the investigator. History of cancer (except treated non-melanoma skin cancer) or history of chemotherapy use within 5 years prior to Screening.
2. Any surgical or medical condition or history that, in the opinion of the investigator, may potentially alter the absorption, metabolism, or excretion of study treatment, such as, but not limited to gastric bypass surgery or gastric or duodenal ulcers.
3. Contraindication to study drugs or its excipients and/or history of allergic or anaphylactic reactions.
4. Taking any of the following prohibited medications:
   a. Any prescription medication or over the counter multi-vitamin supplement, or any non-prescription products (including herbal-containing preparations but excluding acetaminophen) within 14 days prior to Day −1.
b. Any drug known to inhibit or induce cytochrome P450 (CYP) enzymes and/or P-glycoprotein including St. John's wort (Hypericum perforatum) within 14 days or 5 half-lives (whichever is longer) prior to Day −1.
5. History of significant drug abuse within one year prior to screening or use of soft drugs (such as marijuana) within 3 months prior to the screening visit or hard drugs (such as cocaine, phencyclidine [PCP], crack, opioid derivatives including heroin, and amphetamine derivatives) within 1 year prior to screening.
6. History of regular alcohol consumption exceeding 14 drinks/week [1 drink=5 ounces (150 mL) of wine or 12 ounces (360 mL) of beer or 1.5 ounces (45 mL) of hard liquor] within 6 months of Screening.
7. Positive drug and alcohol test at Screening or on Day −1.
8. History of latent or active tuberculosis (TB) as per documented medical history. Exposure to endemic areas within 8 weeks of Screening (for Part 1 and Part 2 only).
9. Positive QuantiFERON®-TB test indicating possible TB infection at Screening (unless documented results of a test performed within 2 months; for Part 1 and Part 2 only).
10. Currently smoke 10 cigarettes or more per day.
11. Consumed any food or drink/beverage containing grapefruit or grapefruit juice, apple or orange juice, pomelo juice, star fruit, Seville or Moro (blood) orange products, and vegetables from the mustard green family (e.g., kale, broccoli, watercress, collard greens, kohlrabi, brussel sprouts, mustard), food containing poppy seeds (e.g., muffins, bagels and cakes) 24 hours before Day −1, and charbroiled meats known to modulate CYP enzyme activity and transporters within 6 days before Day −1.
12. Positive test results of hepatitis B surface antigen (HBsAg), or positive hepatitis C virus antibody (HCVab) test result within 3 months prior to Day −1 or at Screening.
13. Positive test results for human immunodeficiency virus (HIV) antibody within 3 months prior to Day −1 or at Screening.
14. Participation in another clinical trial or exposure to any investigational agent within 30 days or 5 half-lives prior to Day −1, whichever is longer.
15. Donation of plasma within 7 days prior to dosing. Donation or loss of blood (excluding volume drawn at screening) of 50 mL to 499 mL of blood within 30 days, or more than 499 mL within 56 days prior to the first dosing.
16. Received a tattoo or body piercing (including ear piercings) within 2 months prior to Day 1, and/or open wound that may result in risk of infection.
17. Having a condition that the investigator believes would interfere with his/her ability to provide written informed consent, comply with study instructions, or which might confound the interpretation of the study results or put the subject at undue risk.

Criteria for Evaluation

Pharmacokinetics. Blood samples for PK analysis were collected as follow:

In Part 1: For each dose of rosuvastatin administered, blood samples for PK analysis were collected at the following timepoints: pre-dose and 0.5, 1, 2, 3, 4, 4.5, 5, 5.5, 6, 8, 12, 24, 36, 48, 60, 72, 96, and 120 hours postdose.

In Part 2 (Arm 1): For each dose of sulfasalazine administered, blood samples for PK analysis were collected at the following timepoints: pre-dose, 0.5, 1, 2, 3, 4, 5, 6, 8, 12, 18, 24, 36, 48, 60, 72 and 96 hours postdose.

In Part 2 (Arm 2): For each dose of pravastatin administered, blood samples for PK analysis were collected at the following timepoints: pre-dose and 0.5, 1, 1.5, 2, 3, 4, 5, 6, 8, 12, 18, and 24 hours postdose.

In Part 3 (Arm 1): For atorvastatin, on Day 4 and Day 8 of administration, blood samples for PK analysis were collected at the following timepoints: pre-dose, 0.333, 0.667, 1, 1.5, 2, 2.5, 3, 4, 6, 8, 10, 12, 18, and 24 postdose.

In Part 3 (Arm 2): For each dose of simvastatin administered, blood samples for PK analysis were collected at the following timepoints: pre-dose, 0.333, 0.667, 1, 1.5, 2, 2.5, 3, 4, 6, 8, 10, 12, 18, and 24 postdose.

The following PK parameters were calculated for rosuvastatin, sulfasalazine, pravastatin, atorvastatin, simvastatin, and any corresponding metabolites from plasma concentration data for each subject using non-compartmental analysis: AUC₀₋ₗₐₛₜ, AUC_{0-inf}, AUCₜₐᵤ, % AUCₑₓₜᵣₐₚ or Residual Area, CL/F, Cₘₐₓ, Kel, Tₘₐₓ and t_{1/2}.

Safety and Tolerability. Subject safety were evaluated throughout the study beginning on Day 1 (Baseline) until 30 days (±2 days) post last dose. The following safety assessments were performed: physical exam, 12-lead ECG (electrocardiogram), vital signs, clinical laboratory results, QuantiFERON®-TB Test, serology, and pregnancy. Adverse events (AE) were summarized.

Treatment of Subjects

Subjects were randomized into 3 parts as described below in the treatment period.

Part 1 (rosuvastatin)

In Part 1, 34 subjects were treated with the study drug. On the morning of Day 1, subjects received a single oral dose of rosuvastatin 20 mg after at least a 10-hour overnight fast. Subjects would remain fasted for at least 4 hours after dosing. Following a 6-day washout period (Days 1-6) subjects received Compound 1 600 mg once daily (QD) beginning on Day 7 for 8 days (Days 7-14). When administered alone, vadadustat would be administered in the morning at least 30 minutes prior to breakfast. On Day 10, subjects received their morning dose of vadadustat 600 mg along with a single oral dose of rosuvastatin 20 mg after at least a 10-hour overnight fast. Subjects would remain fasted for at least 4 hours after dosing. Subjects were discharged from the clinical research unit (CRU) on the morning of Day 15.

Part 2 (sulfasalazine and pravastatin)

In Part 2, subjects were further randomized into 2 arms as described below:

Arm 1 (sulfasalazine): 26 subjects were treated with the study drug. On the morning of Day 1, subjects received a single oral dose of sulfasalazine 500 mg after at least a 10-hour overnight fast. Subjects would remain fasted for approximately 4 hours after dosing. Following a 5-day washout period (Days 1-5) subjects received vadadustat 600 mg once daily beginning on Day 6 for 7 days (Days 6-12). When administered alone, vadadustat would be administered in the morning at least 30 minutes prior to breakfast. On Day 9, subjects received their morning dose of vadadustat 600 mg along with a single oral dose of sulfasalazine 500 mg after at least a 10-hour overnight fast. Subjects would remain fasted for at least 4 hours after dosing. Subjects were discharged from the clinical research unit on the morning of Day 13.

Arm 2 (pravastatin): 26 subjects were treated with the study drug. On the morning of Day 1, subjects received a single oral dose pravastatin 40 mg after at least a 10-hour overnight fast. Subjects would remain fasted for approximately 4 hours after dosing. Following a 2-day washout period (Days 1-2) subjects received vadadustat 600 mg once daily beginning on Day 3 for 4 days (Days 3-6). When administered alone, vadadustat would be administered in the morning at least 30 minutes prior to breakfast. On Day 6, subjects received their morning dose of vadadustat 600 mg along with a single oral dose of pravastatin 40 mg after at least a 10-hour overnight fast. Subjects would remain fasted for at least 4 hours after dosing. Subjects were discharged from the clinical research unit on the morning of Day 7.

Part 3 (Atorvastatin and Simvastatin)

In Part 3, subjects were further randomized into 2 arms as described below:

Arm 1 (atorvastatin): 24 subjects were treated with the study drug in Arm 1. Beginning on Day 1, subjects received a single oral dose of atorvastatin 40 mg once daily (QD) for 4 days (Days 1-4). On Day 4, subjects received their dose of atorvastatin (40 mg) after at least a 10 hour overnight fast, and would remain fasted for approximately 4 hours after dosing. Beginning on Day 5, subjects received vadadustat 600 mg QD and atorvastatin 40 mg QD for 4 days (Days 5-8). When administered together on Days 5-7, vadadustat and atorvastatin would be administered in the morning at least 30 minutes prior to breakfast. On Day 8, subjects received their dose of vadadustat 600 mg and atorvastatin 40 mg after at least a 10-hour overnight fast, and would remain fasted for at least 4 hours after dosing. Subjects were discharged from the clinical research unit on the morning of Day 9.

Arm 2 (simvastatin): 24 subjects were treated with the study drug in Arm 2. On the morning of Day 1, subjects received a single oral dose simvastatin 40 mg after at least a 10-hour overnight fast. Subjects would remain fasted for approximately 4 hours after dosing. Following a 1-day washout period subjects received vadadustat 600 mg once daily beginning on Day 2 for 3 days (Days 2-4). When administered alone, vadadustat would be administered in the morning at least 30 minutes prior to breakfast. On Day 5, subjects received their morning dose of vadadustat 600 mg along with a single oral dose of simvastatin 40 mg after at least a 10 hour overnight fast. Subjects would remain fasted for at least 4 hours after dosing. Subjects were discharged from the clinical research unit on the morning of Day 6.

Data Analysis

Pharmacokinetic Results for Part 1.

Concentration data for Part 1 subjects who received study drug were included in the PK and statistical analysis. Plasma rosuvastatin PK parameters and statistical analysis results are summarized in Table 14 below.

TABLE 14
Rosuvastatin Pharmacokinetic Parameters (Part 1 subjects)

| | Geometric LSM | | Geometric | 90% Geometric CI | | Intra- |
|---|---|---|---|---|---|---|
| Parameter | Treatment | Treatment | LSM Ratio | Lower | Upper | Subject |
| (unit) | R + V | R | (R + V/R) (%) | (%) | (%) | CV (%) |
| AUC₀₋ₗₐₛₜ | 0.249 | 0.0984 | 252.86 | 231.85 | 275.77 | 21.02 |
| (h · μg/mL) | | | | | | |
| AUC_{0-inf} | 0.254 | 0.103 | 246.86 | 227.08 | 268.36 | 20.23 |
| (h · μg/mL) | | | | | | |
| Cₘₐₓ | 0.0279 | 0.0102 | 274.80 | 246.28 | 306.62 | 26.74 |
| (μg/mL) | | | | | | |

Note:
AUC_{0-inf}: area under the plasma time concentration curve from time 0 to infinity;
AUC₀₋ₗₐₛₜ: area under the plasma time concentration curve from time 0 to the last measurable concentration; CI: confidence interval; Cₘₐₓ: maximal plasma concentration; CV: coefficient of variation; LSM: least squares mean. Treatment R: Rosuvastatin Alone; Treatment R + V: Rosuvastatin + Vadadustat.

Dosage Adjustment of Rosuvastatin

The data described herein indicates that the systemic exposure to rosuvastatin is increased by 2- to 3-fold when rosuvastatin is administered with Compound 1 compared to when rosuvastatin is administered alone. Accordingly, the dosage of rosuvastatin may be adjusted when co-administered with Compound 1, while the dose of Compound 1 (e.g., a daily dose of 150 mg to 600 mg) is maintained. For example, the daily dose of 20 mg rosuvastatin may be decreased by at least about 5 mg or about 10 mg, and a maximum daily dose of 10 mg rosuvastatin is recommended for subjects who are concomitantly taking Compound 1.

Pharmacokinetic Results for Part 2 (Arm 1).

Concentration data for Part 2 (Arm 1) subjects who received study drug were included in the PK and statistical analysis. Plasma PK parameters and statistical analysis results of sulfasalazine, sulfapyridine and mesalamine are summarized in Table 15a, Table 15b, Table 15c, respectively.

TABLE 15a
Sulfasalazine Pharmacokinetic Parameters (Part 2, Arm 1 subjects)

| | Geometric LSM | | Geometric | 90% Geometric CI | | Intra- |
|---|---|---|---|---|---|---|
| Parameter | Treatment | Treatment | LSM Ratio | Lower | Upper | Subject |
| (unit) | S + V | S | (S + V/S) (%) | (%) | (%) | CV (%) |
| AUC₀₋ₗₐₛₜ | 373.01 | 82.17 | 453.94 | 375.51 | 548.75 | 41.70 |
| (h · μg/mL) | | | | | | |
| AUC_{0-inf} | 378.70 | 82.71 | 457.87 | 378.24 | 554.28 | 42.03 |
| (h · μg/mL) | | | | | | |
| Cₘₐₓ | 26.72 | 9.70 | 275.32 | 233.07 | 325.22 | 36.28 |
| (μg/mL) | | | | | | |

Note:
AUC_{0-inf}: area under the plasma time concentration curve from time 0 to infinity;
AUC₀₋ₗₐₛₜ: area under the plasma time concentration curve from time 0 to the last measurable concentration; CI: confidence interval; Cₘₐₓ: maximal plasma concentration; CV: coefficient of variation; LSM: least squares mean. Treatment S: Sulfasalazine Alone; Treatment S + V: Sulfasalazine + Vadadustat

TABLE 15b
Sulfapyridine Pharmacokinetic Parameters (Part 2, Arm 1 subjects)

| | Geometric LSM | | Geometric | 90% Geometric CI | | Intra- |
|---|---|---|---|---|---|---|
| Parameter | Treatment | Treatment | LSM Ratio | Lower | Upper | Subject |
| (unit) | S + V | S | (S + V/S) (%) | (%) | (%) | CV (%) |
| AUC₀₋ₗₐₛₜ | 41.52 | 42.42 | 97.89 | 90.01 | 106.46 | 17.86 |
| (h · μg/mL) | | | | | | |
| AUC_{0-inf} | 42.21 | 42.84 | 98.53 | 90.76 | 106.97 | 17.48 |
| (h · μg/mL) | | | | | | |
| Cₘₐₓ | 2.19 | 2.58 | 84.81 | 77.51 | 92.78 | 19.15 |
| (μg/mL) | | | | | | |

Note:
AUC_{0-inf}: area under the plasma time concentration curve from time 0 to infinity;
AUC₀₋ₗₐₛₜ: area under the plasma time concentration curve from time 0 to the last measurable concentration; CI: confidence interval; Cₘₐₓ: maximal plasma concentration; CV: coefficient of variation; LSM: least squares mean. Treatment S: Sulfasalazine Alone; Treatment S + V: Sulfasalazine + Vadadustat

TABLE 15c
Mesalamine Pharmacokinetic Parameters (Part 2, Arm 1 subjects)

| | Geometric LSM | | Geometric | 90% Geometric CI | | Intra- |
|---|---|---|---|---|---|---|
| Parameter | Treatment | Treatment | LSM Ratio | Lower | Upper | Subject |
| (unit) | S + V | S | (S + V/S) (%) | (%) | (%) | CV (%) |
| AUC₀₋ₗₐₛₜ | 0.318 | 0.220 | 144.43 | 115.56 | 180.51 | 49.81 |
| (h · μg/mL) | | | | | | |
| AUC_{0-inf} | 0.343 | 0.247 | 139.10 | 110.01 | 175.89 | 50.22 |
| (h · μg/mL) | | | | | | |
| Cₘₐₓ | 0.027 | 0.022 | 119.13 | 86.69 | 163.71 | 75.42 |
| (μg/mL) | | | | | | |

Note:
AUC_{0-inf}: area under the plasma time concentration curve from time 0 to infinity;
AUC₀₋ₗₐₛₜ: area under the plasma time concentration curve from time 0 to the last measurable concentration; CI: confidence interval; Cₘₐₓ: maximal plasma concentration; CV: coefficient of variation; LSM: least squares mean. Treatment S: Sulfasalazine Alone; Treatment S + V: Sulfasalazine + Vadadustat

There were minimal changes in the Cₘₐₓ, AUC₀₋ₗₐₛₜ, and AUC_{0-inf} geometric LSM ratios of the active metabolites, sulfapyridine and mesalamine.

Dosage Adjustment of Sulfasalazine

The data described herein indicates that the systemic exposure to sulfasalazine is increased up to 4.5-fold when sulfasalazine is administered with Compound 1 compared to when sulfasalazine is administered alone. Accordingly, the dosage of sulfasalazine may be adjusted when co-administered with Compound 1, while the dose of Compound 1 (e.g., a daily dose of 150 mg to 600 mg) is maintained. For example, the daily dose of 500 mg sulfasalazine may be decreased by at least about 50 mg, about 100 mg, or about 150 mg. Sulfasalazine is recommended to be used with caution when taken concomitantly with Compound 1.

Pharmacokinetic Results for Part 2 (Arm 2).

Concentration data for Part 2 (Arm 2) subjects who received study drug were included in the PK and statistical analysis. Plasma pravastatin PK parameters and statistical analysis results are summarized in Table 16 below.

TABLE 16
Pravastatin Pharmacokinetic Parameters (Part 2, Arm 2 subjects)

| | Geometric LSM | | Geometric | 90% Geometric CI | | Intra- |
|---|---|---|---|---|---|---|
| Parameter | Treatment | Treatment | LSM Ratio | Lower | Upper | Subject |
| (unit) | P + V | P | (P + V/P) (%) | (%) | (%) | CV (%) |
| AUC₀₋ₗₐₛₜ | 134.34 | 130.46 | 102.98 | 92.80 | 114.27 | 21.76 |
| (h · μg/mL) | | | | | | |
| AUC_{0-inf} | 137.00 | 134.27 | 102.03 | 90.87 | 114.56 | 22.60 |
| (h · μg/mL) | | | | | | |
| Cₘₐₓ | 44.01 | 53.19 | 82.74 | 72.27 | 94.73 | 28.53 |
| (μg/mL) | | | | | | |

Note:
AUC_{0-inf}: area under the plasma time concentration curve from time 0 to infinity;
AUC₀₋ₗₐₛₜ: area under the plasma time concentration curve from time 0 to the last measurable concentration; CI: confidence interval; Cₘₐₓ: maximal plasma concentration; CV: coefficient of variation; LSM: least squares mean. Treatment P: Pravastatin Alone; Treatment P + V: Pravastatin + Vadadustat.

Dosage Adjustment of Pravastatin

The data described herein indicates that the systemic exposure to pravastatin was unchanged when pravastatin is administered with Compound 1 compared to when pravastatin was administered alone. Accordingly, no dose adjustment of pravastatin is required when taken concomitantly with Compound 1.

Pharmacokinetic Results for Part 3 (Arm 1).

Concentration data for Part 3 (Arm 1) subjects who received study drug were included in the PK and statistical analysis. Plasma PK parameters and statistical analysis results of atorvastatin, o-hydroxy atorvastatin, and p-hydroxy atorvastatin are summarized in Table 17a, Table 17b, and Table 17c, respectively.

TABLE 17a
Atorvastatin Pharmacokinetic Parameters (Part 3, Arm 1 subjects)

| | Geometric LSM | | Geometric | 90% Geometric CI | | Intra- |
|---|---|---|---|---|---|---|
| Parameter | Treatment | Treatment | LSM Ratio | Lower | Upper | Subject |
| (unit) | A + V | A | (A + V/A) (%) | (%) | (%) | CV (%) |
| AUCₜₐᵤ | 99.50 | 69.73 | 142.69 | 135.94 | 149.78 | 9.82 |
| (h · μg/mL) | | | | | | |
| Cₘₐₓ | 18.05 | 17.97 | 100.45 | 85.30 | 118.30 | 33.98 |
| (μg/mL) | | | | | | |

Note:
AUCₜₐᵤ: area under the plasma time concentration curve during a dosing interval; CI: confidence interval; Cₘₐₓ: maximal plasma concentration; CV: coefficient of variation; LSM: least squares mean.
Treatment A: Atorvastatin Alone; Treatment A + V: Atorvastatin + Vadadustat.

TABLE 17b
o-Hydroxy Atorvastatin Pharmacokinetic Parameters (Part 3, Arm 1 subjects)

| | Geometric LSM | | Ratio | 90% Geometric CI | | Intra- |
|---|---|---|---|---|---|---|
| Parameter | Treatment | Treatment | (A + V/A) | Lower | Upper | Subject |
| (unit) | A + V | A | (%) | (%) | (%) | CV (%) |
| AUCₜₐᵤ | 106.83 | 95.95 | 111.34 | 106.16 | 116.77 | 9.64 |
| (h · μg/mL) | | | | | | |
| Cₘₐₓ | 13.71 | 15.04 | 91.20 | 80.47 | 103.36 | 25.72 |
| (μg/mL) | | | | | | |

Note:
AUCₜₐᵤ: area under the plasma time concentration curve during a dosing interval; CI: confidence interval; Cₘₐₓ: maximal plasma concentration; CV: coefficient of variation; LSM: least squares mean.
Treatment A: Atorvastatin Alone; Treatment A + V: Atorvastatin + Vadadustat.

TABLE 17c
p-Hydroxy Atorvastatin Pharmacokinetic Parameters (Part 3, Arm 1 subjects)

| | Geometric LSM | | Ratio | 90% Geometric CI | | Intra- |
|---|---|---|---|---|---|---|
| Parameter | Treatment | Treatment | (A + V/A) | Lower | Upper | Subject |
| (unit) | A + V | A | (%) | (%) | (%) | CV (%) |
| AUCₜₐᵤ | 15.66 | 8.92 | 175.60 | 162.70 | 189.52 | 15.51 |
| (h · μg/mL) | | | | | | |
| Cₘₐₓ | 1.44 | 0.62 | 230.48 | 192.41 | 276.08 | 37.74 |
| (μg/mL) | | | | | | |

Note:
AUCₜₐᵤ: area under the plasma time concentration curve during a dosing interval; CI: confidence interval; Cₘₐₓ: maximal plasma concentration; CV: coefficient of variation; LSM: least squares mean.
Treatment A: Atorvastatin Alone; Treatment A + V: Atorvastatin + Vadadustat.

Dosage Adjustment of Atorvastatin

The data described herein indicates that while the Cₘₐₓ was unchanged, the total exposure (AUC) was increased about 40% when atorvastatin was administered with Compound 1 compared to when atorvastatin was administered alone. Accordingly, atorvastatin may need to be used with caution and the dosage of atorvastatin may need to be adjusted when co-administered with Compound 1, while the dose of Compound 1 (e.g., a daily dose of 150 mg to 600 mg) is maintained. For example, the daily dose of 40 mg atorvastatin may be decreased by at least about 5 mg, about 10 mg, about 15 mg, or about 20 mg for subjects who are concomitantly taking Compound 1.

Pharmacokinetic Results for Part 3 (Arm 2).

Concentration data for Part 3 (Arm 2) subjects who received study drug were included in the PK and statistical analysis. Plasma PK parameters and statistical analysis results of simvastatin and simvastatin hydroxyl acid (μ-hydroxysimvastatin acid) are summarized in Table 18a and Table 18b, respectively.

TABLE 18a
Simvastatin Pharmacokinetic Parameters (Part 3, Arm 2 subjects)

| | Geometric LSM | | Ratio | 90% Geometric CI | | Intra- |
|---|---|---|---|---|---|---|
| Parameter | Treatment | Treatment | (S + V/S) | Lower | Upper | Subject |
| (unit) | S + V | S | (%) | (%) | (%) | CV (%) |
| AUC₍₀₋ₗₐₛₜ₎ | 59.03 | 29.90 | 197.41 | 174.39 | 223.47 | 24.86 |
| (h · μg/mL) | | | | | | |
| AUC_{(0-inf)} | 63.23 | 32.50 | 194.56 | 169.77 | 222.97 | 27.41 |
| (h · μg/mL) | | | | | | |
| Cₘₐₓ | 11.27 | 9.16 | 123.15 | 104.55 | 145.05 | 33.19 |
| (μg/mL) | | | | | | |

Note:
AUC_{0-inf}: area under the plasma time concentration curve from time 0 to infinity;
AUC₀₋ₗₐₛₜ: area under the plasma time concentration curve from time 0 to the last measurable concentration; CI: confidence interval; Cₘₐₓ: maximal plasma concentration; CV: coefficient of variation; LSM: least squares mean. Treatment S: Simvastatin Alone; Treatment S + V: Simvastatin + Vadadustat.

The total exposure to simvastatin was increased about 2-fold when simvastatin was administered with Compound 1 compared to when simvastatin was administered alone.

TABLE 18b
Hydroxy Simvastatin (β-Hydroxysimvastatin Acid)
Pharmacokinetic Parameters (Part 3, Arm 2 subjects)

| | Geometric LSM | | Ratio | 90% Geometric CI | | Intra- |
|---|---|---|---|---|---|---|
| Parameter | Treatment | Treatment | (S + V/S) | Lower | Upper | Subject |
| (unit) | S + V | S | (%) | (%) | (%) | CV (%) |
| AUC₍₀₋ₗₐₛₜ₎ | 45.56 | 17.33 | 262.90 | 235.56 | 293.42 | 21.95 |
| (h · μg/mL) | | | | | | |
| AUC_{(0-inf)} | 49.63 | 20.16 | 246.21 | 218.73 | 277.15 | 23.11 |
| (h · μg/mL) | | | | | | |
| Cₘₐₓ | 5.39 | 1.85 | 291.84 | 260.40 | 327.07 | 22.80 |
| (μg/mL) | | | | | | |

Note:
AUC_{0-inf}: area under the plasma time concentration curve from time 0 to infinity;
AUC₀₋ₗₐₛₜ: area under the plasma time concentration curve from time 0 to the last measurable concentration; CI: confidence interval; Cₘₐₓ: maximal plasma concentration; CV: coefficient of variation; LSM: least squares mean. Treatment S: Simvastatin Alone; Treatment S + V: Simvastatin + Vadadustat.

Dosage Adjustment of Simvastatin

The data described herein indicates that the systemic exposure to simvastatin is increased 2.5- to 3-fold when simvastatin was administered with Compound 1 compared to when simvastatin was administered alone. Accordingly, the dosage of simvastatin may be adjusted when co-administered with Compound 1, while the dose of Compound 1(e.g., a daily dose of 150 mg to 600 mg) is maintained. For example, the daily dose of 40 mg simvastatin may be decreased by at least about 5 mg, about 10 mg, about 15 mg, or about 20 mg, and a maximum daily dose of 20 mg simvastatin is recommended for subjects who are concomitantly taking Compound 1.

Safety Results.

All abnormal clinical laboratory values, vital signs, and ECG recordings during the study were considered to be not clinically significant and no abnormal physical examination measurements were recorded. Compound 1 was well tolerated, in general, by subjects when taken alone or during treatment with rosuvastatin, pravastatin, atorvastatin, or simvastatin.

### Example 4b: Administration of Furosemide with Compound 1

A phase I, randomized, open-label study was conducted to evaluate the potential for interaction of Compound 1 with Furosemide in 22 healthy male and female subjects. The pharmacokinetic (pK) parameters and safety of said drug administered alone and in combination with Compound 1 were characterized.

The study population consisted of healthy male or female between 18 and 55 years of age, body mass index between 18.0 and 30.0 kg/m², with a minimum body weight of 45 kg for females and 50 kg for males.

Exclusion Criteria:
Current or past clinically significant history of cardiovascular, cerebrovascular, pulmonary, gastrointestinal, hematologic, renal, hepatic, immunologic, metabolic, urologic, neurologic, dermatologic, psychiatric, or other major disease. History of cancer (except treated non-melanoma skin cancer) or history of chemotherapy use within 5 years prior to Screening.
Positive test results for human immunodeficiency virus (HIV) antibody; Positive test results of hepatitis B surface antigen (HBsAg), or positive hepatitis C virus antibody (HCVab) within 3 months prior to screening; or positive test results for human immunodeficiency virus antibody (HIVab) at Screening
Taking any prescription medication or over the counter multi-vitamin supplement, or any non-prescription products (including herbal-containing preparations but excluding acetaminophen) within 14 days prior to Day −1.

The study included 3 periods as follow: a 28-day screening period, 7-21 day in clinic period, and a 30-day follow up period post last dose.

Following the screening period, subjects who meet all inclusion and no exclusion criteria described below were randomized in the subsequent in clinic period.

Treatment of Subjects

On the morning of Day 1, subjects received a single oral 40 mg oral dose of furosemide alone followed by a 2-day washout period. On Days 3 to 6, subjects received vadadustat 600 mg (once daily). A single oral dose of furosemide 40 mg was administered with vadadustat on the morning of Day 6. Serial blood samples were collected after dosing on Day 1 and Day 6 for measurement of plasma furosemide concentrations.

Data Analysis

Concentration data for subjects who received study drug were included in the PK and statistical analysis. Plasma rosuvastatin PK parameters and statistical analysis results are summarized in Table 19 below.

TABLE 19
Furosemide Pharmacokinetic Parameters

| | Geometric LSM | | Geometric | 90% Geometric CI | | Intra- |
|---|---|---|---|---|---|---|
| Parameter | Treatment | Treatment | LSM Ratio | Lower | Upper | Subject |
| (unit) | F + V | R | (F + V/F) (%) | (%) | (%) | CV (%) |
| AUC₀₋ₗₐₛₜ | 5386.54 | 2487.26 | 216.57 | 192.03 | 244.23 | 23.49 |
| (h · μg/mL) | | | | | | |
| AUC_{0-inf} | 5442.49 | 2601.48 | 209.21 | 187.07 | 233.97 | 21.81 |
| (h · μg/mL) | | | | | | |
| Cₘₐₓ | 1464.90 | 855.40 | 171.25 | 136.63 | 214.66 | 45.69 |
| (μg/ml) | | | | | | |

Note:
AUC_{0-inf}: area under the plasma time concentration curve from time 0 to infinity;
AUC₀₋ₗₐₛₜ: area under the plasma time concentration curve from time 0 to the last measurable concentration; CI: confidence interval; _{Cmax}: maximal plasma concentration; CV: coefficient of variation; LSM: least squares mean. Treatment F: Furosemide Alone; Treatment F + V: Furosemide + Vadadustat.

Safety Results: All abnormal clinical laboratory values were considered to be not clinically significant. No serious TEAEs (treatment-emergent adverse events) were reported.

Dosage Adjustment of Furosemide

The data described herein indicates that the systemic exposure to furosemide is increased by approximately 2-fold when furosemide was administered with Compound 1 compared to when furosemide was administered alone. Accordingly, furosemide may need to be used with caution and the dosage of furosemide may need to be adjusted when co-administered with Compound 1, while the dose of Compound 1 (e.g., a daily dose of 150 mg to 600 mg) is maintained. For example, the daily dose of 40 mg furosemide may be decreased by at least about 5 mg, about 10 mg, about 15 mg, or about 20 mg for subjects who are concomitantly taking Compound 1.

### Example 5: Administration of Compound 1 in Subjects with Moderate Hepatic Impairment

As described herein, Compound 1 is primarily metabolized to 0-glucuronide by uridine diphosphate (UDP)-glucuronosyltransferases (UGTs). The predominant UGT involved in the metabolism of vadadustat is UGT1A9, which is expressed in the liver and kidney. Accordingly, there is potential that the pharmacokinetics of Compound 1 may be altered in subjects with hepatic impairment.

A phase I, open-label, parallel-group, single-dose study was conducted to evaluate the pharmacokinetics (PK) of 450 mg vadadustat administered in adults with moderate hepatic impairment (Child-Pugh Class B) versus those with normal hepatic function. The pharmacokinetic (pK) parameters of Compound 1 were characterized. Safety and tolerability were assessed throughout the study.

Inclusion criteria: participants were male and female adults between 18 and 70 years of age, with either normal hepatic function or moderate hepatic impairment based on the Child-Pugh classification (Child-Pugh Class B [score 7-9]). Moderate hepatic impairment was defined by the presence and severity of elevated bilirubin levels, decreased serum albumin levels, elevated international normalized ratios >2.20, ascites, and hepatic encephalopathy.

Exclusion criteria: included history of alcohol or drug abuse, history of liver, other organ, or cell transplant, clinically significant abnormal laboratory findings, recent surgery, and select concomitant medications such as any erythropoiesis-stimulating agents within 30 days prior to dosing. Subjects with renal impairment (eGFR <60 mL/min/1.73 m2) were excluded from this study.

Subjects who meet all inclusion and no exclusion criteria described above may be selected for treatment. Participants in both groups were enrolled to match for gender, race, age (±5 years), weight (±15%), and BMI (±15%).

Criteria for Evaluation

Pharmacokinetics. Blood samples for the evaluation of the PK of Compound 1 and its metabolites were collected predose and at 0.5, 1, 1.5, 2, 3, 4, 6, 9, 12, 18, 24, 36, 48, 60, and 72 hours postdose. Urine was collected predose and at 0, 4, 8, 12, 24, 48, and 72 hours post dose to measure Compound 1 excretion and its O-glucuronide metabolite. The following PK parameters were estimated from the plasma concentration-time profiles based upon actual collections times: Cₘₐₓ, AUC₀₋ₗₐₛₜ, AUC_{0-inf}, Tₘₐₓ, t_{1/2}, CL/F (Clearance), and Vd/F (Volume of distribution). PK parameters Ae₀₋₇₂, Fe %₀₋₇₂, and CL, were estimated from the urine concentration-time profiles.

Safety and Tolerability. Safety assessments included adverse events (AEs), vital signs, clinical laboratory values, electrocardiograms (ECG), and physical examinations.

Treatment of Subjects

8 subjects with hepatic impairment and 8 matched subjects with normal hepatic function were enrolled in the study. All subjects received a single oral 450-mg dose of vadadustat on Day 1 and were required to fast for at least 10 hours before dosing and for 4 hours after dosing.

Data Analysis

Pharmacokinetic Results. Concentration data for all 16 subjects who received study drug were included in the PK and statistical analysis.

Plasma Compound 1 (total and unbound) PK parameters and statistical analysis results are summarized in Table 20 below.

TABLE 20
Pharmacokinetic Parameters

| | Geometric LSM | | | | | |
|---|---|---|---|---|---|---|
| | Moderate | Normal | Geometric | 90% Geometric CI | | Intra- |
| Parameter | Hepatic | Liver | LSM Ratio | Lower | Upper | CV (%) |
| (unit) | Impairment | Function | (T/R) (%) | (%) | (%) | Subject |

Total Compound 1 Parameters

| | | | | | | |
|---|---|---|---|---|---|---|
| AUC₀₋ₗₐₛₜ | 410 | 389 | 105 | 82 | 135 | 29 |
| (h · μg/mL) | | | | | | |
| AUC_{0-inf} | 414 | 394 | 106 | 82 | 136 | 29 |
| (h · μg/mL) | | | | | | |
| Cₘₐₓ | 52 | 50 | 102 | 79 | 132 | 30 |
| (μg/mL) | | | | | | |

Unbound Compound 1 Parameters

| | | | | | | |
|---|---|---|---|---|---|---|
| AUC₀₋ₗₐₛₜ | 3.44 | 2.78 | 124 | 89 | 172 | 39 |
| (h · μg/mL) | | | | | | |
| AUC_{0-inf} | 3.47 | 2.79 | 124 | 89 | 173 | 39 |
| (h · μg/mL) | | | | | | |
| Cₘₐₓ | 0.433 | 0.360 | 120 | 90 | 161 | 34 |
| (μg/mL) | | | | | | |

Note:
AUC_{0-inf}: AUC from dosing (time 0) to infinity; AUC₀₋ₗₐₛₜ: AUC from dosing (time 0) to last quantifiable concentration; Cₘₐₓ: maximum observed plasma concentration; CI: confidence interval; Cₘₐₓ: maximal plasma concentration; CV: coefficient of variation; LSM: least squares mean.

Plasma PK parameters for Compound 1 and Compound 1-O-glucuronide (mean±SD) are summarized in Table 21below.

TABLE 21
Plasma Compound 1 and Compound 1-O-glucuronide

| | Compound 1 | | Compound 1-O-glucuronide | |
|---|---|---|---|---|
| | Moderate | Normal | Moderate | Normal |
| Parameter | Hepatic | Liver | Hepatic | Liver |
| (unit) | Impairment | Function | Impairment | Function |

Total Compound 1 Parameters

| | | | | |
|---|---|---|---|---|
| AUC₀₋ₗₐₛₜ | 432 ± 153 | 395 ± 72 | 68.9 ± 24.2 | 62.5 ± 21.3 |
| (h · μg/mL) | | | | |
| AUC_{0-inf} | 436 ± 156 | 397 ± 72 | 70.9 ± 24.3 | 63.8 ± 21.3 |
| (h · μg/mL) | | | | |
| Cₘₐₓ | 53 ± 12 | 53 ± 15 | 5.85 ± 1.5 | 6.65 ± 2.4 |
| (μg/mL) | | | | |
| Tₘₐₓ | 2.0 | 2.5 | NA | NA |
| (h)^{a} | (1.0, 4.0) | (1.5, 6.0) | | |
| Clearance | 1.2 ± 0.4 | 1.2 ± 0.3 | NA | NA |
| (CL/F) (L/hr) | | | | |
| Volume of | 12.3 ± 4.0 | 9.6 ± 1.9 | NA | NA |
| distribution | | | | |
| (Vd/F) (L) | | | | |
| Elimination Half- | 7.75 ± 2.5 | 5.81 ± 1.4 | 6.01 ± 2.1 | 4.74 ± 1.3 |
| life (t_{1/2}; h) | | | | |
| GM Ratio | 1.05 | NA | NC | NA |
| AUC₀₋ₗₐₛₜ^{b} | (0.82-1.35) | | | |
| GM Ratio | 1.06 | NA | NC | NA |
| AUC_{0-inf}^{b} | (0.82-1.36) | | | |
| GM Ratio | 1.02 | NA | NC | NA |
| Cₘₐₓ^{b} | (0.79-1.32) | | | |

Unbound Compound 1 Parameters

| | | |
|---|---|---|
| AUC₀₋ₗₐₛₜ | 3.75 ± 1.709 | 2.90 ± 0.9001 |
| (h · μg/mL) | | |
| AUC_{0-inf} | 3.78 ± 1.738 | 2.91 ± 0.9049 |
| (h · μg/mL) | | |
| Cₘₐₓ | 0.451 ± 0.1332 | 0.378 ± 0.1131 |
| (μg/mL) | | |
| Volume of | 1490 ± 541.7 | 1330 ± 222.2 |
| distribution | | |
| (Vd/F) (L) | | |
| Clearance | 140 ± 56.02 | 167 ± 49.10 |
| (CL/F) (L/hr) | | |

Note:
AUC_{0-inf}: AUC from dosing (time 0) to infinity; AUC₀₋ₗₐₛₜ: AUC from dosing (time 0) to last quantifiable concentration; Cₘₐₓ: maximum observed plasma concentration; CI: confidence interval; Cₘₐₓ: maximal plasma concentration; NA: not applicable; NC: not calculated. SD: standard deviation;
^{a}Presented as median (minimum, maximum).
^{b}Presented as GM ratio (90% confidence interval).

After a single 450-mg Compound 1 dose, while plasma exposure was numerically higher in subjects with moderate hepatic impairment, plasma total and unbound Compound 1 Cₘₐₓ were comparable for subjects with normal hepatic function and those with moderate hepatic impairment, suggesting that the presence of moderate hepatic impairment had no significant impact on total systemic exposure to Compound 1.

The clearance (CL/F) and volume of distribution (Vd/F) values for Compound 1 were comparable between groups. Median Tₘₐₓ was slightly shorter among subjects with moderate hepatic impairment compared to subjects with normal hepatic function (2.0 vs 2.5 hours), while the mean elimination half-life was slightly longer in subjects with hepatic impairment (7.8 vs 5.8 hours). Plasma Compound 1-O-glucuronide Cₘₐₓ and AUC values were comparable between groups.

Urine PK parameters and statistical analysis results of Compound 1 and Compound 1-O-glucuronide are summarized in Table 22 below.

TABLE 22
Urine Compound 1 and Compound 1-O-glucuronide

| | Compound 1 | | Compound 1-O-glucuronide | |
|---|---|---|---|---|
| | Moderate Hepatic | Normal Liver | Moderate Hepatic | Normal Liver |
| Urine PK | Impairment | Function | Impairment | Function |
| Parameter | (n = 7) | (n = 8) | (n = 8) | (n = 8) |
| Ae₀₋₇₂ (mg) | 2.45 ± 1.611 | 2.76 ± 1.214 | 585 ± 125.1 | 473 ± 83.76 |
| Fe%₀₋₇₂ (%) | 0.545 ± 0.3580 | 0.614 ± 0.2698 | 82.8 ± 17.69 | 66.9 ± 11.84 |
| CLᵣ(mL/h) | 6.30 ± 4.370 | 7.31 ± 3.564 | NA | NA |

Note:
Ae₀₋₇₂: cumulative amount of drug excreted up to 72 hours; CLᵣ, renal clearance; Fe%₀₋₇₂, cumulative fraction of drug excreted PK, pharmacokinetics.

Following a single 450-mg dose of Compound 1, <1% of the dose was excreted in the urine as Compound 1 in both subjects with moderate hepatic impairment (Fe %₀₋₇₂=0.545%) and normal hepatic function (Fe %₀₋₇₂=0.614%). Compound 1 was excreted in urine as Compound 1-O-glucuronide with a slightly higher percentage being excreted in subjects with moderate hepatic impairment (Fe %₀₋₇₂=82.8%) compared to subjects with normal hepatic function (Fe %₀₋₇₂=66.9%).

Safety Results: The incidence and severity of AEs were comparable in both groups. No serious AEs were reported during the study.

The data described herein indicates that moderate hepatic impairment did not alter peak Compound 1 plasma concentrations, nor did it have significant impact on Compound 1 total systemic exposure. Accordingly, patients with mild or moderate hepatic impairment can still benefit from the treatment of Compound 1, and the dose of Compound 1 (e.g., a daily dose of 150 mg to 600 mg) may be maintained in patients with mild or moderate hepatic impairment.

While a number of embodiments of this invention have been described, it is apparent that the basic examples may be altered to provide other embodiments that utilize the compounds, methods, and processes of this invention. Therefore, it will be appreciated that the scope of this invention is to be defined by the appended claims rather than by the specific embodiments that have been represented by way of example herein.

From the ongoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

### EXEMPLARY EMBODIMENTS

### First Set of Embodiments

1. A method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug comprises multivalent cations,
   wherein the subject has renal anemia (anemia secondary to or associated with chronic kidney disease), and
wherein (a) is given at least 1 hour or at least 2 hours before and/or after taking (b).
2. The method of embodiment 2, wherein (b) is a calcium-containing composition, magnesium-containing composition, iron-containing composition, lanthanum-containing composition, or aluminum-containing composition.
3. A method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is an iron-containing composition,
   wherein the subject has renal anemia (anemia secondary to or associated with chronic kidney disease), and
   wherein (a) is given at least 1 hour or at least 2 hours before and/or after taking (b).
4. A method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug comprises multivalent cations,
   wherein the subject has renal anemia (anemia secondary to or associated with chronic kidney disease), and
   wherein (a) is given at least 1 hour or at least 2 hours before and/or after taking (b).
5. The method of embodiment 4, wherein (b) is a calcium-containing composition, magnesium-containing composition, iron-containing composition, lanthanum-containing composition, or aluminum-containing composition.
6. A method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is an iron-containing composition,
   wherein the subject has renal anemia (anemia secondary to or associated with chronic kidney disease), and
   wherein (a) is given at least 1 hour or at least 2 hours before and/or after taking (b).
7. A method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug comprises multivalent cations,
   wherein the subject has renal anemia (anemia secondary to or associated with chronic kidney disease), and wherein (a) is given at least 1 hour or at least 2 hours before and/or after taking (b).
8. The method of embodiment 7, wherein (b) is a calcium-containing composition, magnesium-containing composition, iron-containing composition, lanthanum-containing composition, or aluminum-containing composition.
9. A method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
(b) an effective amount of the second drug, wherein the second drug is an iron-containing composition,
   wherein the subject has renal anemia (anemia secondary to or associated with chronic kidney disease), and
   wherein (a) is given at least 1 hour or at least 2 hours before and/or after taking (b).
10. A method of increasing bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
   wherein the subject has renal anemia (anemia secondary to or associated with chronic kidney disease), and
   wherein (a) is given at least 1 hour or at least 2 hours before and/or after taking (b).
11. The method of embodiment 10, wherein (b) is a calcium-containing composition, magnesium-containing composition, iron-containing composition, lanthanum-containing composition, or aluminum-containing composition.
12. A method of increasing bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
   wherein the subject has renal anemia (anemia secondary to or associated with chronic kidney disease), and
   wherein (a) is given at least 1 hour or at least 2 hours before and/or after taking (b).
13. A method of maintaining bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
   wherein the subject has renal anemia (anemia secondary to or associated with chronic kidney disease), and
   wherein (a) is given at least 1 hour or at least 2 hours before and/or after taking (b).
14. The method of embodiment 13, wherein (b) is a calcium-containing composition, magnesium-containing composition, iron-containing composition, lanthanum-containing composition, or aluminum-containing composition.
15. A method of maintaining bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
   wherein the subject has renal anemia (anemia secondary to or associated with chronic kidney disease), and
   wherein (a) is given at least 1 hour or at least 2 hours before and/or after taking (b).
16. A method of minimizing a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
   wherein the subject has renal anemia (anemia secondary to or associated with chronic kidney disease), and
   wherein (a) is given at least 1 hour or at least 2 hours before and/or after taking (b).
17. The method of embodiment 16, wherein (b) is a calcium-containing composition, magnesium-containing composition, iron-containing composition, lanthanum-containing composition, or aluminum-containing composition.
18. A method of minimizing a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
   wherein the subject has renal anemia (anemia secondary to or associated with chronic kidney disease), and
   wherein (a) is given at least 1 hour or at least 2 hours before and/or after taking (b).
19. A method of preventing a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
   wherein the subject has renal anemia (anemia secondary to or associated with chronic kidney disease), and
   wherein (a) is given at least 1 hour or at least 2 hours before and/or after taking (b).
20. The method of embodiment 19, wherein (b) is a calcium-containing composition, magnesium-containing composition, iron-containing composition, lanthanum-containing composition, or aluminum-containing composition.
21. A method of preventing a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
   wherein the subject has renal anemia (anemia secondary to or associated with chronic kidney disease), and
   wherein (a) is given at least 1 hour or at least 2 hours before and/or after taking (b).
22. A method of controlling a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
   wherein the subject has renal anemia (anemia secondary to or associated with chronic kidney disease), and
   wherein (a) is given at least 1 hour or at least 2 hours before and/or after taking (b).
23. The method of embodiment 22, wherein (b) is a calcium-containing composition, magnesium-containing composition, iron-containing composition, lanthanum-containing composition, or aluminum-containing composition.
24. A method of controlling a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
   wherein the subject has renal anemia (anemia secondary to or associated with chronic kidney disease), and
   wherein (a) is given at least 1 hour or at least 2 hours before and/or after taking (b).
25. A method of controlling drug-multivalent cation chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
   wherein the subject has renal anemia (anemia secondary to or associated with chronic kidney disease), and
   wherein (a) is given at least 1 hour or at least 2 hours before and/or after taking (b).
26. The method of embodiment 25, wherein (b) is a calcium-containing composition, magnesium-containing composition, iron-containing composition, lanthanum-containing composition, or aluminum-containing composition.
27. A method of controlling drug-iron chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
   wherein the subject has renal anemia (anemia secondary to or associated with chronic kidney disease), and
   wherein (a) is given at least 1 hour or at least 2 hours before and/or after taking (b).
28. A method of minimizing or reducing drug-multivalent cation chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
   wherein the subject has renal anemia (anemia secondary to or associated with chronic kidney disease), and
   wherein (a) is given at least 1 hour or at least 2 hours before and/or after taking (b).
29. The method of embodiment 28, wherein (b) is a calcium-containing composition, magnesium-containing composition, iron-containing composition, lanthanum-containing composition, or aluminum-containing composition.
30. A method of minimizing or reducing drug-iron chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
   wherein the subject has renal anemia (anemia secondary to or associated with chronic kidney disease), and
   wherein (a) is given at least 1 hour or at least 2 hours before and/or after taking (b).
31. A method of preventing drug-multivalent cation chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of a drug that is a composition comprising multivalent cations,
   wherein the subject has renal anemia (anemia secondary to or associated with chronic kidney disease), and
wherein (a) is given at least 1 hour or at least 2 hours before and/or after taking (b).
32. The method of embodiment 31, wherein (b) is a calcium-containing composition, magnesium-containing composition, iron-containing composition, lanthanum-containing composition, or aluminum-containing composition.
33. A method of preventing drug-iron chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid; and
(b) an effective amount of an iron-containing composition,
   wherein the subject has renal anemia (anemia secondary to or associated with chronic kidney disease), and
   wherein (a) is given at least 1 hour or at least 2 hours before and/or after taking (b).
34. The method of any one of embodiments 1-33, wherein (a) is given at least 1 hour before and/or after taking (b).
35. The method of any one of embodiments 1-33, wherein (a) is given at least 2 hours before and/or after taking (b).
36. The method of any one of embodiments 1-35, wherein (b) is an iron-containing composition that is oral iron or an iron-containing phosphorus adsorbent.
37. The method of any one of embodiments 1-36, wherein (b) is an iron-containing composition that is an iron-containing phosphorus adsorbent.
38. The method of any one of embodiments 1-36, wherein the iron-containing composition comprises one or more of ferrous sulfate, sodium ferrous citrate, ferric citrate, or sucroferric oxyhydroxide.
39. The method of any one of embodiments 1-38, wherein administration of the iron-containing composition is associated with a medical treatment.
40. The method of any one of embodiments 1-38, wherein the iron-containing composition is administered as a supplement.
41. The method of embodiment 38, wherein the iron-containing composition is administered as a tablet.
42. The method of embodiment 41, wherein the iron-containing composition tablet is a slow release tablet.
43. The method of embodiment 41, wherein the iron-containing composition tablet is a chewable tablet.
44. The method of any one of embodiments 1,2, 4, 5, 7, 8, 10, 11, 13, 14, 16, 17, 19, 20, 22, 23, 25, 26, 28, 29, 31, and 32, wherein (b) is a calcium-containing composition.
45. The method of embodiment 44, wherein (b) is oral calcium acetate.
46. The method of embodiment 44, wherein (b) is oral calcium carbonate.
47. The method of any one of embodiments 1,2, 4, 5, 7, 8, 10, 11, 13, 14, 16, 17, 19, 20, 22, 23, 25, 26, 28, 29, 31, and 32, wherein (b) is a lanthanum-containing composition.
48. The method of embodiment 47, wherein (b) is oral lanthanum carbonate.
49. The method of any one of embodiments 1,2, 4, 5, 7, 8, 10, 11, 13, 14, 16, 17, 19, 20, 22, 23, 25, 26, 28, 29, 31, and 32, wherein (b) is a magnesium-containing composition.
50. The method of any one of embodiments 1,2, 4, 5, 7, 8, 10, 11, 13, 14, 16, 17, 19, 20, 22, 23, 25, 26, 28, 29, 31, and 32, wherein (b) is an aluminum-containing composition.
51. The method of any one of embodiments 1-9, wherein the first drug is given at least 2 hours before taking the second drug.
52. The method of any one of embodiments 1-9, wherein the first drug is given at least 1 hour before taking the second drug.
53. The method of any one of embodiments 1-9, wherein the first drug is given at least 2 hours after taking the second drug.
54. The method of any one of embodiments 10-33, wherein the drug is given at least 2 hours before taking (b) and wherein (b) is an iron-containing composition.
55. The method of any one of embodiments 10-33, wherein the drug is given at least 1 hour before taking (b) and wherein (b) is an iron-containing composition.
56. The method of any one of embodiments 10-33, wherein the drug is given at least 2 hours after taking (b), and wherein (b) is an iron-containing composition.
57. The method of any one of embodiments 10, 11, 13, 14, 16, 17, 19, 20, 22, 23, 25, 26, 28, 29, 31, and 32, wherein the drug is given at least 2 hours before taking (b) and wherein (b) is a calcium-containing composition.
58. The method of any one of embodiments 10, 11, 13, 14, 16, 17, 19, 20, 22, 23, 25, 26, 28, 29, 31, and 32, wherein the drug is given at least 1 hour before taking (b) and wherein (b) is a calcium-containing composition.
59. The method of any one of embodiments 10, 11, 13, 14, 16, 17, 19, 20, 22, 23, 25, 26, 28, 29, 31, and 32, wherein the drug is given at least 2 hours after taking (b), and wherein (b) is a calcium-containing composition.
60. The method of any one of embodiments 10, 11, 13, 14, 16, 17, 19, 20, 22, 23, 25, 26, 28, 29, 31, and 32, wherein the drug is given at least 2 hours before taking (b) and wherein (b) is a lanthanum-containing composition.
61. The method of any one of embodiments 10, 11, 13, 14, 16, 17, 19, 20, 22, 23, 25, 26, 28, 29, 31, and 32, wherein the drug is given at least 1 hour before taking (b) and wherein (b) is a lanthanum-containing composition.
62. The method of any one of embodiments 10, 11, 13, 14, 16, 17, 19, 20, 22, 23, 25, 26, 28, 29, 31, and 32, wherein the drug is given at least 2 hours after taking (b), and wherein (b) is a lanthanum-containing composition.
63. A method of treating renal anemia (anemia secondary to or associated with chronic kidney disease), comprising administering to a subject having renal anemia an effective amount of a compound which is {[5-(3-chlorophenyl)-3-hydroxypyridine-2-carbonyl]amino}acetic acid or pharmaceutically acceptable salt, solvate, or hydrate thereof,
   wherein the compound is for administering together with one or more of oral iron-containing composition selected from sodium ferrous citrate, ferric citrate, and sucroferric oxyhydroxide.
64. The method of embodiment 63, wherein the compound is administered at least 2 hours before and/or after administering the iron-containing composition.
65. The method of embodiment 63, wherein the compound is administered at least 1 hour before administering the iron-containing composition.
66. A method of treating renal anemia (anemia secondary to or associated with chronic kidney disease), comprising administering to a subject having renal anemia an effective amount of a compound which is {[5-(3-chlorophenyl)-3-hydroxypyridine-2-carbonyl]amino}acetic acid or pharmaceutically acceptable salt, solvate, or hydrate thereof, wherein the compound is for administering together with an oral calcium-containing composition that is calcium acetate or calcium carbonate.
67. The method of embodiment 66, wherein the compound is administered at least 2 hours before and/or after administering the calcium-containing composition.
68. The method of embodiment 66, wherein the compound is administered at least 1 hour before administering the calcium-containing composition.
69. A method of treating renal anemia (anemia secondary to or associated with chronic kidney disease), comprising administering to a subject having renal anemia an effective amount of a compound which is {[5-(3-chlorophenyl)-3-hydroxypyridine-2-carbonyl]amino}acetic acid or pharmaceutically acceptable salt, solvate, or hydrate thereof,
   wherein the compound is for administering together with an oral lanthanum-containing composition that is lanthanum carbonate.
70. The method of embodiment 69, wherein the compound is administered at least 2 hours before and/or after administering the lanthanum-containing composition.
71. The method of embodiment 69, wherein the compound is administered at least 1 hour before administering the lanthanum-containing composition.

### Second Set of Embodiments

1. A method of reducing or minimizing drug-drug interaction between a first drug and a second drug comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is a statin drug,
   wherein the subject has anemia secondary to or associated with chronic kidney disease, and
   wherein the amount of (b) is adjusted compared to the amount when administered in the absence of (a) or in monotherapy.
2. A method of preventing drug-drug interaction between a first drug and a second drug comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is a statin drug,
   wherein the subject has anemia secondary to or associated with chronic kidney disease, and
   wherein the amount of (b) is adjusted compared to the amount when administered in the absence of (a) or in monotherapy.
3. A method of controlling drug-drug interaction between a first drug and a second drug comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is a statin drug,
   wherein the subject has anemia secondary to or associated with chronic kidney disease, and
   wherein the amount of (b) is adjusted compared to the amount when administered in the absence of (a) or in monotherapy.
4. A method of maintaining bioavailability of a drug comprising administering to the subject:
   (a) an effective amount of a drug or a pharmaceutical composition comprising an
   effective amount of the drug, wherein the drug is {[5-(3-chlorophenyl)-3-hydroxypyridine-2-carbonyl]amino}acetic acid; and
   (b) an effective amount of a statin drug,
   wherein the subject has anemia secondary to or associated with chronic kidney disease, and
   wherein the amount of (b) is adjusted compared to the amount when administered in the absence of (a) or in monotherapy.
5. A method of minimizing an increase in exposure to a drug comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a statin drug,
   wherein the subject has anemia secondary to or associated with chronic kidney disease, and
   wherein the amount of (b) is adjusted compared to the amount when administered in the absence of (a) or in monotherapy.
6. A method of preventing an increase in exposure to a drug comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a statin drug,
   wherein the subject has anemia secondary to or associated with chronic kidney disease, and
   wherein the amount of (b) is adjusted compared to the amount when administered in the absence of (a) or in monotherapy.
7. A method of controlling an increase in exposure to a drug comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a statin drug,
   wherein the subject has anemia secondary to or associated with chronic kidney disease, and
   wherein the amount of (b) is adjusted compared to the amount when administered in the absence of (a) or in monotherapy.
8. The method of any one of embodiments 1-7, wherein the subject is at risk of or has cardiovascular disease or diabetes.
9. The method of any one of embodiments 1-8, wherein the subject has dyslipidemia.
10. The method of embodiment 9, wherein the subject has elevated cholesterol or elevated triglycerides (hypertriglyceridemia).
11. The method of embodiment 10, wherein the subject has elevated total cholesterol or elevated LDL-cholesterol.
12. The method of embodiment 9, wherein the subject has low HDL-cholesterol.
13. The method of any one of embodiments 1-12, wherein the statin drug of (b) is simvastatin, pitavastatin, fluvastatin, lovastatin, pravastatin, rosuvastatin, or atorvastatin.
14. The methods of any one of embodiments 1-13, wherein the amount of (b) is decreased compared to the amount when administered in the absence of (a) or in monotherapy.
15. The method of embodiment 14, wherein the amount of (b) is decreased by about 20% to about 80% compared to the amount when administered in the absence of (a) or in monotherapy.
16. The method of embodiment 15, wherein the amount of (b) is decreased by about 40% to about 60% compared to the amount when administered in the absence of (a) or in monotherapy.
17. The method of any one of embodiments 1-3 and 8-16, wherein a drug-drug interaction is between Compound 1 and atorvastatin as administered.
18. The method of any one of embodiments 1-3 and 8-16, wherein a drug-drug interaction is between Compound 1 and one or more metabolites of atorvastatin.
19. The method of embodiment 18, wherein a metabolite of atorvastatin is o-hydroxy atorvastatin and/or p-hydroxy atorvastatin.
20. The method of any one of embodiments 5-16, wherein the method relates to the exposure to atorvastatin as administered.
21. The method of any one of embodiments 5-16, wherein the method relates to the exposure to one or more metabolites of atorvastatin.
22. The method of embodiment 21, wherein a metabolite of atorvastatin is o-hydroxy atorvastatin and/or p-hydroxy atorvastatin.
23. The method of any one of embodiments 13-16, wherein the statin is rosuvastatin, and the dose is decreased by at least about 5, or 10 mg.
24. The method of embodiment 23, wherein the maximum daily dose of rosuvastatin is about 10 mg.
25. The method of any one of embodiments 13-16, wherein the statin is simvastatin, and the dose is decreased by at least about 5, 10, or 20 mg.
26. The method of embodiment 25, wherein the maximum daily dose of simvastatin is about 20 mg.
27. The method of any one of embodiments 1-3, 8-16, and 25-26, wherein a drug-drug interaction is between Compound 1 and simvastatin as administered.
28. The method of any one of embodiments 1-3, 8-16, and 25-26, wherein a drug-drug interaction is between Compound 1 and one or more metabolites of simvastatin.
29. The method of embodiment 28, wherein a metabolite of simvastatin is hydroxy simvastatin (P-hydroxysimvastatin acid).
30. The method of any one of embodiments 5-16 and 25-26, wherein the method relates to the exposure to simvastatin as administered.
31. The method of any one of embodiments 5-6 and 25-26, wherein the method relates to the exposure to one or more metabolites of simvastatin.
32. The method of embodiment 31, wherein a metabolite of simvastatin is hydroxy simvastatin (P-hydroxysimvastatin acid).
33. The method of any one of embodiments 13-32, wherein a subject initially receives a statin that is rosuvastatin or simvastatin.
34. The method of embodiment 33, wherein administration of rosuvastatin or simvastatin is discontinued.
35. The method of any one of embodiments 1-34, wherein (b) is administered simultaneously with (a).
36. The method of any one of embodiments 1-34, wherein (b) is not administered simultaneously with (a).
37. The method of any one of embodiments 1-36, wherein the chronic kidney disease is dialysis-dependent chronic kidney disease (DD-CKD).
38. The method of any one of embodiments 1-36, wherein the chronic kidney disease is non dialysis-dependent chronic kidney disease (NDD-CKD).
39. The method of any one of embodiments 1-38, wherein the subject is administered a daily dose of about 150-600 mg of {[5-(3-chlorophenyl)-3-hydroxypyridine-2-carbonyl]amino}acetic acid.
40. The method of embodiment 39, wherein the daily dose of {[5-(3-chlorophenyl)-3-hydroxypyridine-2-carbonyl]amino}acetic acid is increased.
41. The method of any one of embodiments 1-40, wherein the subject is administered an initial daily dose of about 300 mg of {[5-(3-chlorophenyl)-3-hydroxypyridine-2-carbonyl]amino}acetic acid.
42. The method of any one of embodiments 1-40, wherein the subject is administered an initial daily dose of about 450 mg of {[5-(3-chlorophenyl)-3-hydroxypyridine-2-carbonyl]amino}acetic acid.
43. The method of any one of embodiments 1-40, wherein the subject is administered an initial daily dose of about 600 mg of {[5-(3-chlorophenyl)-3-hydroxypyridine-2-carbonyl]amino}acetic acid.
44. A method of reducing or minimizing drug-drug interaction between a first drug and a second drug comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is an anti-inflammatory drug that is sulfasalazine,
   wherein the subject has anemia secondary to or associated with chronic kidney disease, and
   wherein the amount of (b) is adjusted compared to the amount when administered in the absence of (a) or in monotherapy.
45. A method of preventing drug-drug interaction between a first drug and a second drug comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is an anti-inflammatory drug that is sulfasalazine,
   wherein the subject has anemia secondary to or associated with chronic kidney disease, and
   wherein the amount of (b) is adjusted compared to the amount when administered in the absence of (a) or in monotherapy.
46. A method of controlling drug-drug interaction between a first drug and a second drug comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is an anti-inflammatory drug that is sulfasalazine,
   wherein the subject has anemia secondary to or associated with chronic kidney disease, and
   wherein the amount of (b) is adjusted compared to the amount when administered in the absence of (a) or in monotherapy.
47. The method of any one of embodiments 44-46, wherein a drug-drug interaction is between Compound 1 and sulfasalazine as administered.
48. The method of any one of embodiments 44-46, wherein a drug-drug interaction is between Compound 1 and one or more metabolites of sulfasalazine.
49. The method of embodiment 48, wherein a metabolite of sulfasalazine is sulfapyridine and/or mesalamine.
50. A method of maintaining bioavailability of a drug, comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of an anti-inflammatory drug that is sulfasalazine,
   wherein the subject has anemia secondary to or associated with chronic kidney disease, and
   wherein the amount of (b) is adjusted compared to the amount when administered in the absence of (a) or in monotherapy.
51. A method of minimizing an increase in exposure to a drug comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of an anti-inflammatory drug that is sulfasalazine,
   wherein the subject has anemia secondary to or associated with chronic kidney disease, and
   wherein the amount of (b) is adjusted compared to the amount when administered in the absence of (a) or in monotherapy.
52. A method of preventing an increase in exposure to a drug comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of an anti-inflammatory drug that is sulfasalazine,
   wherein the subject has anemia secondary to or associated with chronic kidney disease, and
   wherein the amount of (b) is adjusted compared to the amount when administered in the absence of (a) or in monotherapy.
53. A method of controlling an increase in exposure to a drug comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of an anti-inflammatory drug that is sulfasalazine,
   wherein the subject has anemia secondary to or associated with chronic kidney disease, and
   wherein the amount of (b) is adjusted compared to the amount when administered in the absence of (a) or in monotherapy.
54. The method of any one of embodiments 51-53, wherein the method relates to the exposure to sulfasalazine as administered.
55. The method of any one of embodiments 51-53, wherein the method relates to the exposure to one or more metabolites of sulfasalazine.
56. The method of embodiment 55, wherein a metabolite of sulfasalazine is sulfapyridine and/or mesalamine.
57. The method of any one of embodiments 44-56, wherein the subject is at risk of or has ulcerative colitis, Crohn's disease, or rheumatoid arthritis.
58. The method of any one of embodiments 44-57, wherein (b) is administered simultaneously with (a).
59. The method of any one of embodiments 44-57, wherein (b) is not administered simultaneously with (a).
60. The method of any one of embodiments 44-59, wherein the chronic kidney disease is dialysis-dependent chronic kidney disease (DD-CKD).
61. The method of any one of embodiments 44-59, wherein the chronic kidney disease is non dialysis-dependent chronic kidney disease (NDD-CKD).
62. The method of any one of embodiments 44-61, wherein the subject is administered a daily dose of about 150-600 mg of {[5-(3-chlorophenyl)-3-hydroxypyridine-2-carbonyl]amino}acetic acid.
63. The method of embodiment 62, wherein the daily dose of {[5-(3-chlorophenyl)-3-hydroxypyridine-2-carbonyl]amino}acetic acid is increased.
64. The method of any one of embodiments 44-63, wherein the subject is administered an initial daily dose of about 300 mg of {[5-(3-chlorophenyl)-3-hydroxypyridine-2-carbonyl]amino}acetic acid.
65. The method of any one of embodiments 44-63, wherein the subject is administered an initial daily dose of about 450 mg of {[5-(3-chlorophenyl)-3-hydroxypyridine-2-carbonyl]amino}acetic acid.
66. The method of any one of embodiments 44-63, wherein the subject is administered an initial daily dose of about 600 mg of {[5-(3-chlorophenyl)-3-hydroxypyridine-2-carbonyl]amino}acetic acid.
67. A method of reducing or minimizing drug-drug interaction between a first drug and a second drug comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is a diuretic that is furosemide,
   wherein the subject has anemia secondary to or associated with chronic kidney disease, and
   wherein the amount of (b) is adjusted compared to the amount when administered in the absence of (a) or in monotherapy.
68. A method of preventing drug-drug interaction between a first drug and a second drug comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is a diuretic that is furosemide,
   wherein the subject has anemia secondary to or associated with chronic kidney disease, and
   wherein the amount of (b) is adjusted compared to the amount when administered in the absence of (a) or in monotherapy.
69. A method of controlling drug-drug interaction between a first drug and a second drug comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is a diuretic that is furosemide,
   wherein the subject has anemia secondary to or associated with chronic kidney disease, and
   wherein the amount of (b) is adjusted compared to the amount when administered in the absence of (a) or in monotherapy.
70. A method of maintaining bioavailability of a drug comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a diuretic that is furosemide,
   wherein the subject has anemia secondary to or associated with chronic kidney disease, and
   wherein the amount of (b) is adjusted compared to the amount when administered in the absence of (a) or in monotherapy.
71. A method of minimizing an increase in exposure to a drug comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a diuretic that is furosemide,
   wherein the subject has anemia secondary to or associated with chronic kidney disease, and
   wherein the amount of (b) is adjusted compared to the amount when administered in the absence of (a) or in monotherapy.
72. A method of preventing an increase in exposure to a drug comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a diuretic that is furosemide,
   wherein the subject has anemia secondary to or associated with chronic kidney disease, and
   wherein the amount of (b) is adjusted compared to the amount when administered in the absence of (a) or in monotherapy.
73. A method of controlling an increase in exposure to a drug comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a diuretic that is furosemide,
   wherein the subject has anemia secondary to or associated with chronic kidney disease, and
   wherein the amount of (b) is adjusted compared to the amount when administered in the absence of (a) or in monotherapy.
74. The method of any one of embodiments 67-73, wherein the subject is at risk of or has edema.
75. The method of any one of embodiments 67-73, wherein the subject has cardiovascular disease or liver disease.
76. The method of embodiment 74 or 75, wherein the edema is induced by chronic kidney disease, cardiovascular disease, or liver disease.
77. The method of any one of embodiments 67-76, wherein (b) is administered simultaneously with (a).
78. The method of any one of embodiments 67-76, wherein (b) is not administered simultaneously with (a).
79. The method of any one of embodiments 67-78, wherein the chronic kidney disease is dialysis-dependent chronic kidney disease (DD-CKD).
80. The method of any one of embodiments 67-78, wherein the chronic kidney disease is non dialysis-dependent chronic kidney disease (NDD-CKD).
81. The method of any one of embodiments 67-80, wherein the subject is administered a daily dose of about 150-600 mg of {[5-(3-chlorophenyl)-3-hydroxypyridine-2-carbonyl]amino}acetic acid.
82. The method of embodiment 81, wherein the daily dose of {[5-(3-chlorophenyl)-3-hydroxypyridine-2-carbonyl]amino}acetic acid is increased.
83. The method of any one of embodiments 67-82, wherein the subject is administered an initial daily dose of about 300 mg of {[5-(3-chlorophenyl)-3-hydroxypyridine-2-carbonyl]amino}acetic acid.
84. The method of any one of embodiments 67-82, wherein the subject is administered an initial daily dose of about 450 mg of {[5-(3-chlorophenyl)-3-hydroxypyridine-2-carbonyl]amino}acetic acid.
85. The method of any one of embodiments 67-82, wherein the subject is administered an initial daily dose of about 600 mg of {[5-(3-chlorophenyl)-3-hydroxypyridine-2-carbonyl]amino}acetic acid.
86. A method of treating anemia associated with or secondary to chronic kidney disease in a subject with hepatic impairment, comprising administering to the subject an effective amount of {[5-(3-chlorophenyl)-3-hydroxypyridine-2-carbonyl]amino}acetic acid, or a pharmaceutically acceptable salt thereof.
87. The method of embodiment 86, wherein the hepatic impairment is mild or moderate hepatic impairment.
88. The method of embodiment 87, wherein the hepatic impairment is characterized as Child Pugh Class B (7-9 points).
89. The method of any one of embodiments 86-88, wherein the hepatic impairment is characterized by elevated bilirubin levels, decreased serum albumin levels, elevated international normalized ratios (INR), ascites, and/or hepatic encephalopathy.
90. The method of embodiment 89, wherein the elevated international normalized ratio (INR) is >2.20.
91. The method of any one of embodiments 86-90, wherein the chronic kidney disease is dialysis-dependent chronic kidney disease (DD-CKD).
92. The method of any one of embodiments 86-90, wherein the chronic kidney disease is non dialysis-dependent chronic kidney disease (NDD-CKD).
93. The method of any one of embodiments 86-92, wherein the subject is administered a daily dose of about 150-600 mg of {[5-(3-chlorophenyl)-3-hydroxypyridine-2-carbonyl]amino}acetic acid.
94. The method of embodiment 93, wherein the daily dose of {[5-(3-chlorophenyl)-3-hydroxypyridine-2-carbonyl]amino}acetic acid is increased.
95. The method of any one of embodiments 86-94, wherein the subject is administered an initial daily dose of about 300 mg of {[5-(3-chlorophenyl)-3-hydroxypyridine-2-carbonyl]amino}acetic acid.
96. The method of any one of embodiments 86-94, wherein the subject is administered an initial daily dose of about 450 mg of {[5-(3-chlorophenyl)-3-hydroxypyridine-2-carbonyl]amino}acetic acid.
97. A method of reducing or minimizing drug-drug interaction between a first drug and a second drug or a metabolite thereof, comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is a BCRP (breast cancer resistance protein) substrate,
   wherein the subject has anemia secondary to or associated with chronic kidney disease, and
   wherein the amount of (b) is adjusted compared to the amount when administered in the absence of (a) or in monotherapy.
98. A method of preventing drug-drug interaction between a first drug and a second drug or a metabolite thereof, comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is a BCRP (breast cancer resistance protein) substrate,
   wherein the subject has anemia secondary to or associated with chronic kidney disease, and
   wherein the amount of (b) is adjusted compared to the amount when administered in the absence of (a) or in monotherapy.
99. A method of controlling drug-drug interaction between a first drug and a second drug or a metabolite thereof, comprising administering to a subject:
   (a) an effective amount of the first drug or a pharmaceutical composition comprising an effective amount of the first drug, wherein the first drug is {[5-(3-chlorophenyl)-3-hydroxypyridine-2-carbonyl]amino}acetic acid; and
   (b) an effective amount of the second drug, wherein the second drug is a BCRP (breast cancer resistance protein) substrate,
   wherein the subject has anemia secondary to or associated with chronic kidney disease, and
   wherein the amount of (b) is adjusted compared to the amount when administered in the absence of (a) or in monotherapy.
100. A method of maintaining bioavailability of a drug or a metabolite thereof, comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a drug that is a BCRP (breast cancer resistance protein) substrate,
   wherein the subject has anemia secondary to or associated with chronic kidney disease, and
   wherein the amount of (b) is adjusted compared to the amount when administered in the absence of (a) or in monotherapy.
101. A method of minimizing an increase in exposure to a drug or a metabolite thereof, comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a drug that is a BCRP (breast cancer resistance protein) substrate,
   wherein the subject has anemia secondary to or associated with chronic kidney disease, and
   wherein the amount of (b) is adjusted compared to the amount when administered in the absence of (a) or in monotherapy.
102. A method of preventing an increase in exposure to a drug or a metabolite thereof, comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a drug that is a BCRP (breast cancer resistance protein) substrate,
   wherein the subject has anemia secondary to or associated with chronic kidney disease, and
   wherein the amount of (b) is adjusted compared to the amount when administered in the absence of (a) or in monotherapy.
103. A method of controlling an increase in exposure to a drug or a metabolite thereof, comprising administering to a subject:
   (a) an effective amount of a drug or a pharmaceutical composition comprising an effective amount of the drug, wherein the drug is {[5-(3-chlorophenyl)-3-hydroxypyridine-2-carbonyl]amino}acetic acid; and
   (b) an effective amount of a drug that is a BCRP (breast cancer resistance protein) substrate,
   wherein the subject has anemia secondary to or associated with chronic kidney disease, and
   wherein the amount of (b) is adjusted compared to the amount when administered in the absence of (a) or in monotherapy.
104. The method of any one of embodiments 97-103, wherein the drug that is a BCRP (breast cancer resistance protein) substrate is mitoxantrone, imatinib, irinotecan, lapatinib, apixaban, atorvastatin, baricitinib, copanlisib, dolutegravir, eltrombopag, ethinylertradiol, glecaprevir, glyburide, letermovir, methotrexate, paritaprevir, pibrentasvir, pravastatin, presatovir, prucalopride, rosuvastatin, simvastatin, sofosbuvir, sulfasalazine, tenofovir, topotecan, velpatasvir, venetoclax, or voxilaprevir.
105. The method of any one of embodiments 97-104, wherein the drug that is a BCRP (breast cancer resistance protein) substrate is atorvastatin, pravastatin, rosuvastatin, simvastatin, or sulfasalazine.
106. The method of any one of embodiments 97-105, wherein (b) is administered simultaneously with (a).
107. The method of any one of embodiments 97-105, wherein (b) is not administered simultaneously with (a).
108. The method of any one of embodiments 97-107, wherein the chronic kidney disease is dialysis-dependent chronic kidney disease (DD-CKD).
109. The method of any one of embodiments 97-107, wherein the chronic kidney disease is non dialysis-dependent chronic kidney disease (NDD-CKD).
110. The method of any one of embodiments 97-109, wherein the subject is administered a daily dose of about 150-600 mg of {[5-(3-chlorophenyl)-3-hydroxypyridine-2-carbonyl]amino}acetic acid.
111. The method of embodiment 110, wherein the daily dose of {[5-(3-chlorophenyl)-3-hydroxypyridine-2-carbonyl]amino}acetic acid is increased.
112. The method of any one of embodiments 97-111, wherein the subject is administered an initial daily dose of about 300 mg of {[5-(3-chlorophenyl)-3-hydroxypyridine-2-carbonyl]amino}acetic acid.
113. The method of any one of embodiments 97-111, wherein the subject is administered an initial daily dose of about 450 mg of {[5-(3-chlorophenyl)-3-hydroxypyridine-2-carbonyl]amino}acetic acid.
114. The method of any one of embodiments 97-111, wherein the subject is administered an initial daily dose of about 600 mg of {[5-(3-chlorophenyl)-3-hydroxypyridine-2-carbonyl]amino}acetic acid.
115. A method of treating anemia associated with or secondary to chronic kidney disease in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving a drug that is a Breast Cancer Resistance Protein (BCRP) substrate.
116. The method of embodiment 115, wherein the BCRP substrate is mitoxantrone, imatinib, irinotecan, lapatinib, apixaban, atorvastatin, baricitinib, copanlisib, dolutegravir, eltrombopag, ethinylertradiol, glecaprevir, glyburide, letermovir, methotrexate, paritaprevir, pibrentasvir, pravastatin, presatovir, prucalopride, rosuvastatin, simvastatin, sofosbuvir, sulfasalazine, tenofovir, topotecan, velpatasvir, venetoclax, or voxilaprevir.
117. A method of treating anemia associated with or secondary to chronic kidney disease in a subject, comprising administering to the subject an effective amount of {[5-(3-chlorophenyl)-3-hydroxypyridine-2-carbonyl]amino}acetic acid, or a pharmaceutically acceptable salt thereof,
   wherein the subject is receiving a diuretic that is furosemide.
118. A method of treating anemia associated with or secondary to chronic kidney disease in a subject, comprising administering to the subject an effective amount of {[5-(3-chlorophenyl)-3-hydroxypyridine-2-carbonyl]amino}acetic acid, or a pharmaceutically acceptable salt thereof, wherein the subject is receiving a statin drug.
119. The method of embodiment 118, wherein the statin is simvastatin, pitavastatin, fluvastatin, lovastatin, pravastatin, rosuvastatin, or atorvastatin.
120. A method of treating anemia associated with or secondary to chronic kidney disease in a subject, comprising administering to the subject an effective amount of {[5-(3-chlorophenyl)-3-hydroxypyridine-2-carbonyl]amino}acetic acid, or a pharmaceutically acceptable salt thereof,
   wherein the subject is receiving sulfasalazine.
121. The method of embodiment 115-120, wherein the chronic kidney disease is dialysis-dependent chronic kidney disease (DD-CKD).
122. The method of embodiment 115-120, wherein the chronic kidney disease is non dialysis-dependent chronic kidney disease (NDD-CKD).
123. The method of any one of embodiments 115-122, wherein the subject is administered a daily dose of about 150-600 mg of {[5-(3-chlorophenyl)-3-hydroxypyridine-2-carbonyl]amino}acetic acid.
124. The method of embodiment 123, wherein the daily dose of {[5-(3-chlorophenyl)-3-hydroxypyridine-2-carbonyl]amino}acetic acid is increased.
125. The method of any one of embodiments 115-124, wherein the subject is administered an initial daily dose of about 300 mg of {[5-(3-chlorophenyl)-3-hydroxypyridine-2-carbonyl]amino}acetic acid.
126. The method of any one of embodiments 115-124, wherein the subject is administered an initial daily dose of about 450 mg of {[5-(3-chlorophenyl)-3-hydroxypyridine-2-carbonyl]amino}acetic acid.
127. The method of any one of embodiments 115-124, wherein the subject is administered an initial daily dose of about 600 mg of {[5-(3-chlorophenyl)-3-hydroxypyridine-2-carbonyl]amino}acetic acid.

What is claimed is:

1. A method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug comprises multivalent cations,
wherein the subject has renal anemia (anemia secondary to or associated with chronic kidney disease), and
wherein (a) is given at least 1 hour or at least 2 hours before and/or after taking (b).

2. The method of claim 1, wherein (b) is a calcium-containing composition, magnesium-containing composition, iron-containing composition, lanthanum-containing composition, or aluminum-containing composition.

3. A method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is an iron-containing composition,
wherein the subject has renal anemia (anemia secondary to or associated with chronic kidney disease), and
wherein (a) is given at least 1 hour or at least 2 hours before and/or after taking (b).

4. A method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug comprises multivalent cations,
wherein the subject has renal anemia (anemia secondary to or associated with chronic kidney disease), and
wherein (a) is given at least 1 hour or at least 2 hours before and/or after taking (b).

5. The method of claim 4, wherein (b) is a calcium-containing composition, magnesium-containing composition, iron-containing composition, lanthanum-containing composition, or aluminum-containing composition.

6. A method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is an iron-containing composition,
wherein the subject has renal anemia (anemia secondary to or associated with chronic kidney disease), and
wherein (a) is given at least 1 hour or at least 2 hours before and/or after taking (b).

7. A method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug comprises multivalent cations,
wherein the subject has renal anemia (anemia secondary to or associated with chronic kidney disease), and
wherein (a) is given at least 1 hour or at least 2 hours before and/or after taking (b).

8. The method of claim 7, wherein (b) is a calcium-containing composition, magnesium-containing composition, iron-containing composition, lanthanum-containing composition, or aluminum-containing composition.

9. A method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is an iron-containing composition,
wherein the subject has renal anemia (anemia secondary to or associated with chronic kidney disease), and
wherein (a) is given at least 1 hour or at least 2 hours before and/or after taking (b).

10. A method of increasing bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

11. The method of claim 10, wherein (b) is a calcium-containing composition, magnesium-containing composition, iron-containing composition, lanthanum-containing composition, or aluminum-containing composition.

12. A method of increasing bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

13. A method of maintaining bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

14. The method of claim 13, wherein (b) is a calcium-containing composition, magnesium-containing composition, iron-containing composition, lanthanum-containing composition, or aluminum-containing composition.

15. A method of maintaining bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

16. A method of minimizing a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

17. The method of claim 16, wherein (b) is a calcium-containing composition, magnesium-containing composition, iron-containing composition, lanthanum-containing composition, or aluminum-containing composition.

18. A method of minimizing a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

19. A method of preventing a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

20. The method of claim 19, wherein (b) is a calcium-containing composition, magnesium-containing composition, iron-containing composition, lanthanum-containing composition, or aluminum-containing composition.

21. A method of preventing a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

22. A method of controlling a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

23. The method of claim 22, wherein (b) is a calcium-containing composition, magnesium-containing composition, iron-containing composition, lanthanum-containing composition, or aluminum-containing composition.

24. A method of controlling a decrease in the absorption of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

25. A method of controlling drug-multivalent cation chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

26. The method of claim 25, wherein (b) is a calcium-containing composition, magnesium-containing composition, iron-containing composition, lanthanum-containing composition, or aluminum-containing composition.

27. A method of controlling drug-iron chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

28. A method of minimizing or reducing drug-multivalent cation chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

29. The method of claim 28, wherein (b) is a calcium-containing composition, magnesium-containing composition, iron-containing composition, lanthanum-containing composition, or aluminum-containing composition.

30. A method of minimizing or reducing drug-iron chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

31. A method of preventing drug-multivalent cation chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a composition comprising multivalent cations,
wherein the subject has renal anemia (anemia secondary to or associated with chronic kidney disease), and
wherein (a) is given at least 1 hour or at least 2 hours before and/or after taking (b).

32. The method of claim 31, wherein (b) is a calcium-containing composition, magnesium-containing composition, iron-containing composition, lanthanum-containing composition, or aluminum-containing composition.

33. A method of preventing drug-iron chelate formation comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an iron-containing composition,
wherein the subject has renal anemia (anemia secondary to or associated with chronic kidney disease), and
wherein (a) is given at least 1 hour or at least 2 hours before and/or after taking (b).

34. The method of any one of claims 1-33, wherein (a) is given at least 1 hour before and/or after taking (b).

35. The method of any one of claims 1-33, wherein (a) is given at least 2 hours before and/or after taking (b).

36. The method of any one of claims 1-35, wherein (b) is an iron-containing composition that is oral iron or an iron-containing phosphorus adsorbent.

37. The method of any one of claims 1-36, wherein (b) is an iron-containing composition that is an iron-containing phosphorus adsorbent.

38. The method of any one of claims 1-36, wherein the iron-containing composition comprises one or more of ferrous sulfate, sodium ferrous citrate, ferric citrate, or sucroferric oxyhydroxide.

39. The method of any one of claims 1-38, wherein administration of the iron-containing composition is associated with a medical treatment.

40. The method of any one of claims 1-38, wherein the iron-containing composition is administered as a supplement.

41. The method of claim 38, wherein the iron-containing composition is administered as a tablet.

42. The method of claim 41, wherein the iron-containing composition tablet is a slow release tablet.

43. The method of claim 41, wherein the iron-containing composition tablet is a chewable tablet.

44. The method of any one of claims 1, 2, 4, 5, 7, 8, 10, 11, 13, 14, 16, 17, 19, 20, 22, 23, 25, 26, 28, 29, 31, and 32, wherein (b) is a calcium-containing composition.

45. The method of claim 44, wherein (b) is oral calcium acetate.

46. The method of claim 44, wherein (b) is oral calcium carbonate.

47. The method of any one of claims 1, 2, 4, 5, 7, 8, 10, 11, 13, 14, 16, 17, 19, 20, 22, 23, 25, 26, 28, 29, 31, and 32, wherein (b) is a lanthanum-containing composition.

48. The method of claim 47, wherein (b) is oral lanthanum carbonate.

49. The method of any one of claims 1, 2, 4, 5, 7, 8, 10, 11, 13, 14, 16, 17, 19, 20, 22, 23, 25, 26, 28, 29, 31, and 32, wherein (b) is a magnesium-containing composition.

50. The method of any one of claims 1, 2, 4, 5, 7, 8, 10, 11, 13, 14, 16, 17, 19, 20, 22, 23, 25, 26, 28, 29, 31, and 32, wherein (b) is an aluminum-containing composition.

51. The method of any one of claims 1-9, wherein the first drug is given at least 2 hours before taking the second drug.

52. The method of any one of claims 1-9, wherein the first drug is given at least 1 hour before taking the second drug.

53. The method of any one of claims 1-9, wherein the first drug is given at least 2 hours after taking the second drug.

54. The method of any one of claims 10-33, wherein the drug is given at least 2 hours before taking (b) and wherein (b) is an iron-containing composition.

55. The method of any one of claims 10-33, wherein the drug is given at least 1 hour before taking (b) and wherein (b) is an iron-containing composition.

56. The method of any one of claims 10-33, wherein the drug is given at least 2 hours after taking (b), and wherein (b) is an iron-containing composition.

57. The method of any one of claims 10, 11, 13, 14, 16, 17, 19, 20, 22, 23, 25, 26, 28, 29, 31, and 32, wherein the drug is given at least 2 hours before taking (b) and wherein (b) is a calcium-containing composition.

58. The method of any one of claims 10, 11, 13, 14, 16, 17, 19, 20, 22, 23, 25, 26, 28, 29, 31, and 32, wherein the drug is given at least 1 hour before taking (b) and wherein (b) is a calcium-containing composition.

59. The method of any one of claims 10, 11, 13, 14, 16, 17, 19, 20, 22, 23, 25, 26, 28, 29, 31, and 32, wherein the drug is given at least 2 hours after taking (b), and wherein (b) is a calcium-containing composition.

60. The method of any one of claims 10, 11, 13, 14, 16, 17, 19, 20, 22, 23, 25, 26, 28, 29, 31, and 32, wherein the drug is given at least 2 hours before taking (b) and wherein (b) is a lanthanum-containing composition.

61. The method of any one of claims 10, 11, 13, 14, 16, 17, 19, 20, 22, 23, 25, 26, 28, 29, 31, and 32, wherein the drug is given at least 1 hour before taking (b) and wherein (b) is a lanthanum-containing composition.

62. The method of any one of claims 10, 11, 13, 14, 16, 17, 19, 20, 22, 23, 25, 26, 28, 29, 31, and 32, wherein the drug is given at least 2 hours after taking (b), and wherein (b) is a lanthanum-containing composition.

63. A method of treating renal anemia (anemia secondary to or associated with chronic kidney disease), comprising administering to a subject having renal anemia an effective amount of a compound which is {[5-(3-chlorophenyl)-3-hydroxypyridine-2-carbonyl]amino}acetic acid (Compound 1) or pharmaceutically acceptable salt, solvate, or hydrate thereof,
wherein the compound is for administering together with one or more of oral iron-containing composition selected from sodium ferrous citrate, ferric citrate, and sucroferric oxyhydroxide.

64. The method of claim 63, wherein the compound is administered at least 2 hours before and/or after administering the iron-containing composition.

65. The method of claim 63, wherein the compound is administered at least 1 hour before administering the iron-containing composition.

66. A method of treating renal anemia (anemia secondary to or associated with chronic kidney disease), comprising administering to a subject having renal anemia an effective amount of a compound which is {[5-(3-chlorophenyl)-3-hydroxypyridine-2-carbonyl]amino}acetic acid (Compound 1) or pharmaceutically acceptable salt, solvate, or hydrate thereof,
wherein the compound is for administering together with an oral calcium-containing composition that is calcium acetate or calcium carbonate.

67. The method of claim 66, wherein the compound is administered at least 2 hours before and/or after administering the calcium-containing composition.

68. The method of claim 66, wherein the compound is administered at least 1 hour before administering the calcium-containing composition.

69. A method of treating renal anemia (anemia secondary to or associated with chronic kidney disease), comprising administering to a subject having renal anemia an effective amount of a compound which is {[5-(3-chlorophenyl)-3-hydroxypyridine-2-carbonyl]amino}acetic acid (Compound 1) or pharmaceutically acceptable salt, solvate, or hydrate thereof,
wherein the compound is for administering together with an oral lanthanum-containing composition that is lanthanum carbonate.

70. The method of claim 69, wherein the compound is administered at least 2 hours before and/or after administering the lanthanum-containing composition.

71. The method of claim 69, wherein the compound is administered at least 1 hour before administering the lanthanum-containing composition.

72. A method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is a statin drug,
wherein the subject has renal anemia (anemia secondary to or associated with chronic kidney disease), and
wherein the amount of (b) is adjusted compared to the amount when administered in the absence of (a) or in monotherapy.

73. A method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is a statin drug,
wherein the subject has renal anemia (anemia secondary to or associated with chronic kidney disease), and
wherein the amount of (b) is adjusted compared to the amount when administered in the absence of (a) or in monotherapy.

74. A method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is a statin drug,
wherein the subject has renal anemia (anemia secondary to or associated with chronic kidney disease), and
wherein the amount of (b) is adjusted compared to the amount when administered in the absence of (a) or in monotherapy.

75. A method of maintaining bioavailability of a drug comprising administering to the subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a statin drug,
wherein the subject has renal anemia (anemia secondary to or associated with chronic kidney disease), and
wherein the amount of (b) is adjusted compared to the amount when administered in the absence of (a) or in monotherapy.

76. A method of minimizing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a statin drug,
wherein the subject has renal anemia (anemia secondary to or associated with chronic kidney disease), and
wherein the amount of (b) is adjusted compared to the amount when administered in the absence of (a) or in monotherapy.

77. A method of preventing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a statin drug,
wherein the subject has renal anemia (anemia secondary to or associated with chronic kidney disease), and
wherein the amount of (b) is adjusted compared to the amount when administered in the absence of (a) or in monotherapy.

78. A method of controlling an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a statin drug,
wherein the subject has renal anemia (anemia secondary to or associated with chronic kidney disease), and
wherein the amount of (b) is adjusted compared to the amount when administered in the absence of (a) or in monotherapy.

79. The method of any one of claims 72-78, wherein the subject is at risk of or has cardiovascular disease or diabetes.

80. The method of any one of claims 72-78, wherein the subject has dyslipidemia.

81. The method of claim 80, wherein the subject has elevated cholesterol or elevated triglycerides (hypertriglyceridemia).

82. The method of claim 81, wherein the subject has elevated total cholesterol or elevated LDL-cholesterol.

83. The method of claim 80, wherein the subject has low HDL-cholesterol.

84. The method of any one of claims 72-83, wherein the statin drug of (b) is simvastatin, pitavastatin, fluvastatin, lovastatin, pravastatin, rosuvastatin, or atorvastatin.

85. The methods of any one of claims 72-84, wherein the amount of (b) is decreased compared to the amount when administered in the absence of (a) or in monotherapy.

86. The method of claim 85, wherein the amount of (b) is decreased by about 20% to about 80% compared to the amount when administered in the absence of (a) or in monotherapy.

87. The method of claim 86, wherein the amount of (b) is decreased by about 40% to about 60% compared to the amount when administered in the absence of (a) or in monotherapy.

88. The method of any one of claims 72-74 and 79-87, wherein the drug-drug interaction is between {[5-(3-chlorophenyl)-3-hydroxypyridine-2-carbonyl]amino}acetic acid (Compound 1) and atorvastatin as administered.

89. The method of any one of claims 72-74 and 79-87, wherein the drug-drug interaction is between {[5-(3-chlorophenyl)-3-hydroxypyridine-2-carbonyl]amino}acetic acid (Compound 1) and one or more metabolites of atorvastatin.

90. The method of claim 89, wherein the metabolite of atorvastatin is o-hydroxy atorvastatin and/or p-hydroxy atorvastatin.

91. The method of any one of claims 76-87, wherein the method relates to the exposure to atorvastatin as administered.

92. The method of any one of claims 76-87, wherein the method relates to the exposure to one or more metabolites of atorvastatin.

93. The method of claim 92, wherein the metabolite of atorvastatin is o-hydroxy atorvastatin and/or p-hydroxy atorvastatin.

94. The method of any one of claims 84-87, wherein the statin drug is rosuvastatin, and the dose is decreased by at least about 5, or 10 mg.

95. The method of claim 94, wherein rosuvastatin is administered at a maximum daily dose of about 10 mg.

96. The method of any one of claims 84-87, wherein the statin drug is simvastatin, and the dose is decreased by at least about 5, 10, or 20 mg.

97. The method of claim 96, wherein simvastatin is administered at a maximum daily dose of about 20 mg.

98. The method of any one of claims 72-74, 79-87, 96, and 97, wherein the drug-drug interaction is between {[5-(3-chlorophenyl)-3-hydroxypyridine-2-carbonyl]amino}acetic acid (Compound 1) and simvastatin as administered.

99. The method of any one of claims 72-74, 79-87, 96, and 97, wherein the drug-drug interaction is between {[5-(3-chlorophenyl)-3-hydroxypyridine-2-carbonyl]amino}acetic acid (Compound 1) and one or more metabolites of simvastatin.

100. The method of claim 99, wherein the metabolite of simvastatin is β-hydroxysimvastatin acid.

101. The method of any one of claims 76-87, 96, and 97, wherein the method relates to the exposure to simvastatin as administered.

102. The method of any one of claims 76-87, 96, and 97, wherein the method relates to the exposure to one or more metabolites of simvastatin.

103. The method of claim 102, wherein the metabolite of simvastatin is β-hydroxysimvastatin acid.

104. The method of any one of claims 84-103, wherein the subject initially receives a statin drug that is rosuvastatin or simvastatin.

105. The method of claim 104, wherein administration of rosuvastatin or simvastatin is discontinued.

106. The method of any one of claims 72-105, wherein (b) is administered simultaneously with (a).

107. The method of any one of claims 72-105, wherein (b) is not administered simultaneously with (a).

108. The method of any one of claims 1-107, wherein the chronic kidney disease is dialysis-dependent chronic kidney disease (DD-CKD).

109. The method of any one of claims 1-107, wherein the chronic kidney disease is non dialysis-dependent chronic kidney disease (NDD-CKD).

110. The method of any one of claims 1-109, wherein the subject is administered a daily dose of about 150-600 mg of {[5-(3-chlorophenyl)-3-hydroxypyridine-2-carbonyl]amino}acetic acid (Compound 1).

111. The method of claim 110, wherein the daily dose of {[5-(3-chlorophenyl)-3-hydroxypyridine-2-carbonyl]amino}acetic acid (Compound 1) is increased.

112. The method of any one of claims 1-111, wherein the subject is administered an initial daily dose of about 300 mg of {[5-(3-chlorophenyl)-3-hydroxypyridine-2-carbonyl]amino}acetic acid (Compound 1).

113. The method of any one of claims 1-111, wherein the subject is administered an initial daily dose of about 450 mg of {[5-(3-chlorophenyl)-3-hydroxypyridine-2-carbonyl]amino}acetic acid (Compound 1).

114. The method of any one of claims 1-111, wherein the subject is administered an initial daily dose of about 600 mg of {[5-(3-chlorophenyl)-3-hydroxypyridine-2-carbonyl]amino}acetic acid (Compound 1).

115. A method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

116. A method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

117. A method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

118. The method of any one of claims 115-117, wherein the drug-drug interaction is between {[5-(3-chlorophenyl)-3-hydroxypyridine-2-carbonyl]amino}acetic acid (Compound 1) and sulfasalazine as administered.

119. The method of any one of claims 115-117, wherein the drug-drug interaction is between {[5-(3-chlorophenyl)-3-hydroxypyridine-2-carbonyl]amino}acetic acid (Compound 1) and one or more metabolites of sulfasalazine.

120. The method of claim 119, wherein the metabolite of sulfasalazine is sulfapyridine and/or mesalamine.

121. A method of maintaining bioavailability of a drug, comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

122. A method of minimizing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

123. A method of preventing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

124. A method of controlling an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of an anti-inflammatory drug that is sulfasalazine,
wherein the subject has renal anemia (anemia secondary to or associated with chronic kidney disease), and
wherein the amount of (b) is adjusted compared to the amount when administered in the absence of (a) or in monotherapy.

125. The method of any one of claims 122-124, wherein the method relates to the exposure to sulfasalazine as administered.

126. The method of any one of claims 122-124, wherein the method relates to the exposure to one or more metabolites of sulfasalazine.

127. The method of claim 126, wherein the metabolite of sulfasalazine is sulfapyridine and/or mesalamine.

128. The method of any one of claims 115-127, wherein the subject is at risk of or has ulcerative colitis, Crohn's disease, or rheumatoid arthritis.

129. The method of any one of claims 115-128, wherein (b) is administered simultaneously with (a).

130. The method of any one of claims 115-128, wherein (b) is not administered simultaneously with (a).

131. The method of any one of claims 115-130, wherein the chronic kidney disease is dialysis-dependent chronic kidney disease (DD-CKD).

132. The method of any one of claims 115-130, wherein the chronic kidney disease is non dialysis-dependent chronic kidney disease (NDD-CKD).

133. The method of any one of claims 115-132, wherein the subject is administered a daily dose of about 150-600 mg of {[5-(3-chlorophenyl)-3-hydroxypyridine-2-carbonyl]amino}acetic acid (Compound 1).

134. The method of claim 133, wherein the daily dose of {[5-(3-chlorophenyl)-3-hydroxypyridine-2-carbonyl]amino}acetic acid (Compound 1) is increased.

135. The method of any one of claims 115-134, wherein the subject is administered an initial daily dose of about 300 mg of {[5-(3-chlorophenyl)-3-hydroxypyridine-2-carbonyl]amino}acetic acid (Compound 1).

136. The method of any one of claims 115-134, wherein the subject is administered an initial daily dose of about 450 mg of {[5-(3-chlorophenyl)-3-hydroxypyridine-2-carbonyl]amino}acetic acid (Compound 1).

137. The method of any one of claims 115-134, wherein the subject is administered an initial daily dose of about 600 mg of {[5-(3-chlorophenyl)-3-hydroxypyridine-2-carbonyl]amino}acetic acid (Compound 1).

138. A method of reducing or minimizing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is a diuretic that is furosemide,
wherein the subject has renal anemia (anemia secondary to or associated with chronic kidney disease), and
wherein the amount of (b) is adjusted compared to the amount when administered in the absence of (a) or in monotherapy.

139. A method of preventing drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is a diuretic that is furosemide,
wherein the subject has renal anemia (anemia secondary to or associated with chronic kidney disease), and
wherein the amount of (b) is adjusted compared to the amount when administered in the absence of (a) or in monotherapy.

140. A method of controlling drug-drug interaction between a first drug and a second drug comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is a diuretic that is furosemide,
wherein the subject has renal anemia (anemia secondary to or associated with chronic kidney disease), and
wherein the amount of (b) is adjusted compared to the amount when administered in the absence of (a) or in monotherapy.

141. A method of maintaining bioavailability of a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a diuretic that is furosemide,
wherein the subject has renal anemia (anemia secondary to or associated with chronic kidney disease), and
wherein the amount of (b) is adjusted compared to the amount when administered in the absence of (a) or in monotherapy.

142. A method of minimizing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a diuretic that is furosemide,
wherein the subject has renal anemia (anemia secondary to or associated with chronic kidney disease), and
wherein the amount of (b) is adjusted compared to the amount when administered in the absence of (a) or in monotherapy.

143. A method of preventing an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a diuretic that is furosemide,
wherein the subject has renal anemia (anemia secondary to or associated with chronic kidney disease), and
wherein the amount of (b) is adjusted compared to the amount when administered in the absence of (a) or in monotherapy.

144. A method of controlling an increase in exposure to a drug comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a diuretic that is furosemide,
wherein the subject has renal anemia (anemia secondary to or associated with chronic kidney disease), and
wherein the amount of (b) is adjusted compared to the amount when administered in the absence of (a) or in monotherapy.

145. The method of any one of claims 138-144, wherein the subject is at risk of or has edema.

146. The method of any one of claims 138-144, wherein the subject has cardiovascular disease or liver disease.

147. The method of claim 145, wherein the edema is induced by chronic kidney disease, cardiovascular disease, or liver disease.

148. The method of any one of claims 138-147, wherein (b) is administered simultaneously with (a).

149. The method of any one of claims 138-147, wherein (b) is not administered simultaneously with (a).

150. The method of any one of claims 138-149, wherein the chronic kidney disease is dialysis-dependent chronic kidney disease (DD-CKD).

151. The method of any one of claims 138-149, wherein the chronic kidney disease is non dialysis-dependent chronic kidney disease (NDD-CKD).

152. The method of any one of claims 138-151, wherein the subject is administered a daily dose of about 150-600 mg of {[5-(3-chlorophenyl)-3-hydroxypyridine-2-carbonyl]amino}acetic acid (Compound 1).

153. The method of claim 152, wherein the daily dose of {[5-(3-chlorophenyl)-3-hydroxypyridine-2-carbonyl]amino}acetic acid is increased.

154. The method of any one of claims 138-153, wherein the subject is administered an initial daily dose of about 300 mg of {[5-(3-chlorophenyl)-3-hydroxypyridine-2-carbonyl]amino}acetic acid (Compound 1).

155. The method of any one of claims 138-153, wherein the subject is administered an initial daily dose of about 450 mg of {[5-(3-chlorophenyl)-3-hydroxypyridine-2-carbonyl]amino}acetic acid (Compound 1).

156. The method of any one of claims 138-153, wherein the subject is administered an initial daily dose of about 600 mg of {[5-(3-chlorophenyl)-3-hydroxypyridine-2-carbonyl]amino}acetic acid (Compound 1).

157. A method of treating renal anemia (anemia associated with or secondary to chronic kidney disease) in a subject with hepatic impairment, comprising administering to the subject an effective amount of {[5-(3-chlorophenyl)-3-hydroxypyridine-2-carbonyl]amino}acetic acid (Compound 1), or a pharmaceutically acceptable salt thereof.

158. The method of claim 157, wherein the hepatic impairment is mild or moderate hepatic impairment.

159. The method of claim 158, wherein the hepatic impairment is characterized as Child Pugh Class B (7-9 points).

160. The method of any one of claims 157-159, wherein the hepatic impairment is characterized by elevated bilirubin levels, decreased serum albumin levels, elevated international normalized ratios (INR), ascites, and/or hepatic encephalopathy.

161. The method of claim 160, wherein the elevated international normalized ratio (INR) is >2.20.

162. The method of any one of claims 157-161, wherein the chronic kidney disease is dialysis-dependent chronic kidney disease (DD-CKD).

163. The method of any one of claims 157-161, wherein the chronic kidney disease is non dialysis-dependent chronic kidney disease (NDD-CKD).

164. The method of any one of claims 157-163, wherein the subject is administered a daily dose of about 150-600 mg of {[5-(3-chlorophenyl)-3-hydroxypyridine-2-carbonyl]amino}acetic acid (Compound 1).

165. The method of claim 164, wherein the daily dose of {[5-(3-chlorophenyl)-3-hydroxypyridine-2-carbonyl]amino}acetic acid (Compound 1) is increased.

166. The method of any one of claims 157-165, wherein the subject is administered an initial daily dose of about 300 mg of {[5-(3-chlorophenyl)-3-hydroxypyridine-2-carbonyl]amino}acetic acid (Compound 1).

167. The method of any one of claims 157-165, wherein the subject is administered an initial daily dose of about 450 mg of {[5-(3-chlorophenyl)-3-hydroxypyridine-2-carbonyl]amino}acetic acid (Compound 1).

168. A method of reducing or minimizing drug-drug interaction between a first drug and a second drug or a metabolite thereof, comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

169. A method of preventing drug-drug interaction between a first drug and a second drug or a metabolite thereof, comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

170. A method of controlling drug-drug interaction between a first drug and a second drug or a metabolite thereof, comprising administering to a subject:
(a) an effective amount of the first drug or a pharmaceutical composition comprising an effective amount of the first drug, wherein the first drug is {[5-(3-chlorophenyl)-3-hydroxypyridine-2-carbonyl]amino}acetic acid (Compound 1); and
(b) an effective amount of the second drug, wherein the second drug is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

171. A method of maintaining bioavailability of a drug or a metabolite thereof, comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

172. A method of minimizing an increase in exposure to a drug or a metabolite thereof, comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

173. A method of preventing an increase in exposure to a drug or a metabolite thereof, comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

174. A method of controlling an increase in exposure to a drug or a metabolite thereof, comprising administering to a subject:
(a) an effective amount of a drug or a pharmaceutical composition comprising an effective amount of the drug, wherein the drug is {[5-(3-chlorophenyl)-3-hydroxypyridine-2-carbonyl]amino}acetic acid (Compound 1); and
(b) an effective amount of a drug that is a BCRP (breast cancer resistance protein) substrate,
wherein the subject has renal anemia (anemia secondary to or associated with chronic kidney disease), and
wherein the amount of (b) is adjusted compared to the amount when administered in the absence of (a) or in monotherapy.

175. The method of any one of claims 168-174, wherein the drug that is a BCRP (breast cancer resistance protein) substrate is mitoxantrone, imatinib, irinotecan, lapatinib, apixaban, atorvastatin, baricitinib, copanlisib, dolutegravir, eltrombopag, ethinylertradiol, glecaprevir, glyburide, letermovir, methotrexate, paritaprevir, pibrentasvir, pravastatin, presatovir, prucalopride, rosuvastatin, simvastatin, sofosbuvir, sulfasalazine, tenofovir, topotecan, velpatasvir, venetoclax, or voxilaprevir.

176. The method of any one of claims 168-175, wherein the drug that is a BCRP (breast cancer resistance protein) substrate is atorvastatin, pravastatin, rosuvastatin, simvastatin, or sulfasalazine.

177. The method of any one of claims 168-176, wherein (b) is administered simultaneously with (a).

178. The method of any one of claims 168-176, wherein (b) is not administered simultaneously with (a).

179. The method of any one of claims 168-178, wherein the chronic kidney disease is dialysis-dependent chronic kidney disease (DD-CKD).

180. The method of any one of claims 168-178, wherein the chronic kidney disease is non dialysis-dependent chronic kidney disease (NDD-CKD).

181. The method of any one of claims 168-180, wherein the subject is administered a daily dose of about 150-600 mg of {[5-(3-chlorophenyl)-3-hydroxypyridine-2-carbonyl]amino}acetic acid (Compound 1).

182. The method of claim 181, wherein the daily dose of {[5-(3-chlorophenyl)-3-hydroxypyridine-2-carbonyl]amino}acetic acid (Compound 1) is increased.

183. The method of any one of claims 168-182, wherein the subject is administered an initial daily dose of about 300 mg of {[5-(3-chlorophenyl)-3-hydroxypyridine-2-carbonyl]amino}acetic acid (Compound 1).

184. The method of any one of claims 168-182, wherein the subject is administered an initial daily dose of about 450 mg of {[5-(3-chlorophenyl)-3-hydroxypyridine-2-carbonyl]amino}acetic acid (Compound 1).

185. The method of any one of claims 168-182, wherein the subject is administered an initial daily dose of about 600 mg of {[5-(3-chlorophenyl)-3-hydroxypyridine-2-carbonyl]amino}acetic acid (Compound 1).

186. A method of treating renal anemia (anemia associated with or secondary to chronic kidney disease) in a subject, comprising administering to the subject an effective amount of {[5-(3-chlorophenyl)-3-hydroxypyridine-2-carbonyl]amino}acetic acid (Compound 1), or a pharmaceutically acceptable salt thereof,
wherein the subject is receiving a drug that is a Breast Cancer Resistance Protein (BCRP) substrate.

187. The method of claim 186, wherein the BCRP substrate is mitoxantrone, imatinib, irinotecan, lapatinib, apixaban, atorvastatin, baricitinib, copanlisib, dolutegravir, eltrombopag, ethinylertradiol, glecaprevir, glyburide, letermovir, methotrexate, paritaprevir, pibrentasvir, pravastatin, presatovir, prucalopride, rosuvastatin, simvastatin, sofosbuvir, sulfasalazine, tenofovir, topotecan, velpatasvir, venetoclax, or voxilaprevir.

188. A method of treating renal anemia (anemia associated with or secondary to chronic kidney disease) in a subject, comprising administering to the subject an effective amount of {[5-(3-chlorophenyl)-3-hydroxypyridine-2-carbonyl]amino}acetic acid (Compound 1), or a pharmaceutically acceptable salt thereof,
wherein the subject is receiving a diuretic that is furosemide.

189. A method of treating renal anemia (anemia associated with or secondary to chronic kidney disease) in a subject, comprising administering to the subject an effective amount of {[5-(3-chlorophenyl)-3-hydroxypyridine-2-carbonyl]amino}acetic acid (Compound 1), or a pharmaceutically acceptable salt thereof,
wherein the subject is receiving a statin drug.

190. The method of claim 189, wherein the statin drug is simvastatin, pitavastatin, fluvastatin, lovastatin, pravastatin, rosuvastatin, or atorvastatin.

191. A method of treating renal anemia (anemia associated with or secondary to chronic kidney disease) in a subject, comprising administering to the subject an effective amount of {[5-(3-chlorophenyl)-3-hydroxypyridine-2-carbonyl]amino}acetic acid (Compound 1), or a pharmaceutically acceptable salt thereof,
wherein the subject is receiving sulfasalazine.

192. The method of claim 186-191, wherein the chronic kidney disease is dialysis-dependent chronic kidney disease (DD-CKD).

193. The method of claim 186-191, wherein the chronic kidney disease is non dialysis-dependent chronic kidney disease (NDD-CKD).

194. The method of any one of claims 186-193, wherein the subject is administered a daily dose of about 150-600 mg of {[5-(3-chlorophenyl)-3-hydroxypyridine-2-carbonyl]amino}acetic acid (Compound 1).

195. The method of claim 194, wherein the daily dose of {[5-(3-chlorophenyl)-3-hydroxypyridine-2-carbonyl]amino}acetic acid (Compound 1) is increased.

196. The method of any one of claims 186-195, wherein the subject is administered an initial daily dose of about 300 mg of {[5-(3-chlorophenyl)-3-hydroxypyridine-2-carbonyl]amino}acetic acid (Compound 1).

197. The method of any one of claims 186-195, wherein the subject is administered an initial daily dose of about 450 mg of {[5-(3-chlorophenyl)-3-hydroxypyridine-2-carbonyl]amino}acetic acid (Compound 1).

198. The method of any one of claims 186-195, wherein the subject is administered an initial daily dose of about 600 mg of {[5-(3-chlorophenyl)-3-hydroxypyridine-2-carbonyl]amino}acetic acid (Compound 1).
